(19) United States
(12) Patent Application Publication
(10) Pub. No.: US 2026/0258467 A1
(43) Pub. Date: Sep. 3, 2026

(54) ABC TRANSPORTERS FOR THE HIGH EFFICIENCY PRODUCTION OF REBAUDIOSIDES

(71) Applicant: AMYRIS, INC., Emeryville, CA (US)

(72) Inventors: Gale A. WICHMANN, Emeryville, CA (US); Sean LUND, Emeryville, CA (US); Joshua LERMAN, Emeryville, CA (US); Hanxiao JIANG, Emeryville, CA (US); Yi XIONG, Emeryville, CA (US)

(21) Appl. No.: 19/420,323

(22) Filed: Dec. 15, 2025

Related U.S. Application Data
(63) Continuation of application No. 17/425,634, filed on Jan. 23, 2020, now Pat. No. 12,529,087.
(60) Provisional application No. 62/796,228, filed on Jan. 24, 2019.

(51) Int. Cl. C12P 19/56; C12N 9/02; C12N 9/10; C12N 9/88; C12N 9/90; C12P 5/00; C12P 7/40

(52) U.S. Cl. CPC C12P 19/56; C12N 9/0042; C12N 9/0071; C12N 9/0073; C12N 9/1051; C12N 9/1085; C12N 9/88; C12N 9/90; C12P 5/007; C12P 7/40; C12Y 106/02004; C12Y 114/13079; C12Y 204/01017; C12Y 205/01001; C12Y 402/03019; C12Y 505/01013

(57) ABSTRACT

Provided herein are genetically modified host cells, compositions, and methods for improved production of steviol glycosides. In some embodiments, the host cell is genetically modified to comprise a heterologous nucleic acid expression cassette that expresses an ABC-transporter capable of transporting steviol glycosides to the extracellular space or to the luminal space of an intracellular organelle. In some embodiments, the host cell further comprises one or more heterologous nucleotide sequence encoding further enzymes of a pathway capable of producing one or more steviol glycosides in the host cell. The host cells, compositions, and methods described herein provide an efficient route for the heterologous production of steviol glycosides, including but not limited to, rebaudioside D and rebaudioside M.

### 1. CROSS-REFERENCE TO RELATED APPLICATION(S)

The present application is a continuation application of U.S. application Ser. No. 17/425,634 filed on Jul. 23, 2021, issued as U.S. Pat. No. 12,529,087, which claims the benefit of provisional U.S. Patent Application Ser. No. 62/796,228 filed Jan. 24, 2019, entitled “ABC TRANSPORTERS FOR THE HIGH EFFICIENCY PRODUCTION OF REBAUDIOSIDES,” the disclosure of all of which is hereby incorporated by reference into the present application.

### INCORPORATION-BY-REFERENCE OF SEQUENCE LISTING

The application contains a Sequence Listing which has been submitted electronically in .XML format and is hereby incorporated by reference in its entirety. Said .XML copy, created on Dec. 29, 2025, is named “107345.00971.xml” and is 98,790 bytes in size. The sequence listing contained in this .XML file is part of the specification and is hereby incorporated by reference herein in its entirety.

### 2. FIELD OF THE INVENTION

The present disclosure relates to particular ABC-transporters, host cells comprising the same, and methods of their use for the production of steviol and/or rebaudiosides including rebaudioside D and rebaudioside M.

### 3. BACKGROUND

Reduced-calorie sweeteners derived from natural sources are desired to limit the health effects of high-sugar consumption. The stevia plant (Stevia rebaudiana Bertoni) produces a variety of sweet-tasting glycosylated diterpenes termed steviol glycosides. Of all the known steviol glycosides, Reb M has the highest potency (~200-300× sweeter than sucrose) and has the most appealing flavor profile. However, Reb M is only produced in minute quantities by the stevia plant and is a small fraction of the total steviol glycoside content (<1.0%), making the isolation of Reb M from stevia leaves impractical. Alternative methods of obtaining Reb M are needed. One such approach is the application of synthetic biology to design microorganisms (e.g. yeast) that produce large quantities of Reb M from sustainable feedstock sources.

To economically produce a product using synthetic biology, each step in the bioconversion from feedstock to product needs to have a high conversion efficiency (ideally >90%). In our engineering of yeast to produce Reb M, we noted that cytosolic accumulation of Reb M repressed the steviol glycoside metabolic pathway engineered into the yeast, thereby limiting the total yield of a fermentation run. This repression is likely due to product inhibition or end-product inhibition of one or more enzymes involved in steviol glycoside biosynthesis. Accordingly, novel mechanisms of relieving the product inhibition are needed to increase the conversion efficiency of biosynthetic Reb M production.

### 4. SUMMARY OF THE INVENTION

Provided herein are genetically modified host cells, compositions, and methods for the improved production of Reb M. These compositions and methods are based in part on the expression of certain heterologous ABC-transporters in host cells that have been genetically modified to produce steviol glycosides such as Reb M. These ABC-transporters are capable of transporting certain steviol glycosides, preferably Reb M and/or the related high molecular weight steviol glycoside rebaudioside D (Reb D), out of the cytosol either into the extracellular space or into the lumen of subcellular organelles, for example the yeast vacuole. The sequestration of certain steviol glycosides like Reb D and Reb M increases the efficiency of the steviol glycoside metabolic pathway by relieving the product inhibition caused by the accumulation of steviol glycosides.

In one aspect of the invention, provided herein are genetically modified host cells and methods of their use for the production of industrially useful compounds. In one aspect, provided herein is a genetically modified host cell capable of producing one or more steviol glycosides where the host cell contains a heterologous nucleic acid encoding an ABC-transporter having an amino acid sequence having at least 80% sequence identity to an amino acid sequence selected from the sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID: 28, SEQ ID NO: 29, and SEQ ID NO: 30.

In one embodiment of the invention the ABC-transporter has an amino acid sequence having a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 28, SEQ ID NO: 29, and SEQ ID NO: 30. In another embodiment the genetically modified host cells of the invention contain nucleic acids encoding geranylgeranyl pyrophosphate synthase (GGPPS), ent-copalyl pyrophosphate synthase (CPS), ent-kaurene synthase (KS), ent-kaurene 19-oxidase (KO), ent-kaurenoic acid 13-hydroxylase (KAH), cytochrome p450 reductase (CPR), and one or more UDP-glucosyltransferases (UGT). In a further embodiment the one or more UDP-glucosyltransferases (UGT) are selected from EUGT11, UGT85C2, UGT74G1, UGT91D_like3, UGT76G1, and UGT40087. In a further embodiment of the invention the geranylgeranyl pyrophosphate synthase (GGPPS) has an amino acid sequence having at least 80% sequence identity to SEQ ID NO: 9, the ent-copalyl pyrophosphate synthase (CPS) has an amino acid sequence having at least 80% sequence identity to SEQ ID NO: 10, the ent-kaurene synthase (KS) has an amino acid sequence having at least 80% sequence identity to SEQ ID NO: 11, the ent-kaurene 19-oxidase (KO) has an amino acid sequence having at least 80% sequence identity to SEQ ID NO: 12, the ent-kaurenoic acid 13-hydroxylase (KAH) has an amino acid sequence having at least 80% sequence identity to SEQ ID NO: 13, the cytochrome p450 reductase (CPR) has an amino acid sequence having at least 80% sequence identity to SEQ ID NO: 14, and the one or more UDP-glucosyltransferases (UGT) has an amino acid sequence having at least 80% sequence identity to an amino acid sequence selected from the group consisting of SEQ ID NO: 15, SEQ ID NO: 16, SEQ ID NO: 17, SEQ ID NO:18, SEQ ID NO: 19, SEQ ID NO: 27.

In a particular embodiment of the invention the geranylgeranyl pyrophosphate synthase (GGPPS) has an amino acid sequence of SEQ ID NO: 9, the ent-copalyl pyrophosphate synthase (CPS) has an amino acid sequence of SEQ ID NO: 10, the ent-kaurene synthase (KS) has an amino acid sequence of SEQ ID NO: 11, the ent-kaurene 19-oxidase (KO) comprises an amino acid sequence of SEQ ID NO: 12, the ent-kaurenoic acid 13-hydroxylase (KAH) comprises an amino acid sequence of SEQ ID NO: 13, the cytochrome p450 reductase (CPR) comprises an amino acid sequence of SEQ ID NO: 14, and the one or more UDP-glucosyltransferases (UGT) comprise an amino acid sequence selected from the group consisting of SEQ ID NO: 15, SEQ ID NO: 16, SEQ ID NO: 17, SEQ ID NO:18, SEQ ID NO: 19, and SEQ ID NO: 27.

In an embodiment the host cell is selected from a bacterial cell, a fungal cell, an algal cell, an insect cell, and a plant cell. In another embodiment the host cell is a Saccharomyces cerevisiae cell.

In an embodiment of the invention the ABC-transporter has an amino acid sequence having the sequence of SEQ ID NO: 1.

In another embodiment the ABC-transporter has an amino acid sequence having the sequence of SEQ ID NO: 2.

In a further embodiment the ABC-transporter has an amino acid sequence having the sequence of SEQ ID NO: 3.

In yet another embodiment the ABC-transporter has an amino acid sequence having the sequence of SEQ ID NO: 4.

In additional embodiment the ABC-transporter has an amino acid sequence having the sequence of SEQ ID NO: 5.

In an embodiment the ABC-transporter has an amino acid sequence having the sequence of SEQ ID NO: 6.

In another embodiment the ABC-transporter has an amino acid sequence having the sequence of SEQ ID NO: 7.

In yet another embodiment the ABC-transporter has an amino acid sequence having the sequence of SEQ ID NO: 8

In yet another embodiment the ABC-transporter has an amino acid sequence having the sequence of SEQ ID NO: 28.

In yet another embodiment the ABC-transporter has an amino acid sequence having the sequence of SEQ ID NO: 29.

In yet another embodiment the ABC-transporter has an amino acid sequence having the sequence of SEQ ID NO: 30.

In an embodiment of the invention the one or more steviol glycosides is selected from rebaudioside A (Reb A), rebaudioside B (Reb B), Reb D, rebaudioside E (Reb E), or Reb M. In another embodiment the one or more steviol glycosides comprises Reb M.

In one embodiment a majority of the one or more steviol glycosides accumulate within a lumen of an organelle. In another embodiment a majority of the one or more steviol glycosides accumulate extracellularly.

In another aspect the invention provides a nucleic acid sequence of a heterologous nucleic acid expression cassette that expresses an ABC-transporter. In an embodiment the nucleotide sequence of the heterologous nucleic acid expression cassette has a coding sequence of SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27, where the coding sequence is operably linked to a heterologous promoter.

In another aspect the invention provides for a method for producing steviol or one or more steviol glycosides involving: culturing a population of the host cells of the invention in a medium with a carbon source under conditions suitable for making steviol or one or more steviol glycosides to yield a culture broth; and recovering the steviol or one or more steviol glycosides from the culture broth.

In another aspect the invention provides for a method for producing Reb D involving: culturing a population of the host cells of the invention in a medium with a carbon source under conditions suitable for making Reb D to yield a culture broth; and recovering said Reb D compound from the culture broth.

In another aspect the invention provides for a method for producing Reb M involving: culturing a population of the host cells of the invention in a medium with a carbon source under conditions suitable for making Reb M to yield a culture broth; and recovering said Reb M compound from the culture broth.

### 5. BRIEF DESCRIPTION OF THE FIGURES

FIG. 1 is a schematic showing an enzymatic pathway from the native yeast metabolite famesyl pyrophosphate (FPP) to steviol.

FIG. 2 is a schematic showing an enzymatic pathway from steviol to Rebaudioside M.

FIG. 3 is a schematic of the landing pad DNA construct used to insert transporters into Reb M strains. Each end of the construct contains 500 bp of DNA sequence from downstream of the yeast SFM1 gene to facilitate homologous recombination at this locus. Insertion of the landing pad at this locus does not delete any gene. The landing pad contains a full length GAL1 promoter followed by a recognition site for the F-CphI endonuclease and the terminator from the native yeast gene HEM13.

FIG. 4 is a graph of the percent of Reb D+Reb M found in the supernatant. Yeast strains with different overexpressed transporters were grown in microtiter plates. This figure reports the percent of Reb D+Reb M (measured in pmoles) that is detected in the supernatant after the cells have been removed. The parent strain does not contain an overexpressed transporter. The amount of Reb D+Reb M measured in the supernatant is divided by the amount of Reb D+Reb M measured in the whole cell broth to obtain the percent of Reb D+Reb M in the supernatant.

FIG. 5 is a graph of total steviol glycosides relative to parent in whole cell broth. Yeast strains with different overexpressed transporters were grown in microtiter plates. This figure reports the sum total of all steviol glycosides (measured in pmoles) that is detected in whole cell broth (both cells and supernatant) relative to the parent strain. The parent strain does not contain an overexpressed transporter.

FIG. 6 is a graph of the amount of Reb D+Reb M relative to parent in whole cell broth. Yeast strains with different overexpressed transporters were grown in microtiter plates. This figure reports the sum of Reb D+Reb M (measured in pmoles) that is detected in whole cell broth (both cells and supernatant) relative to the parent strain. The parent strain does not contain an overexpressed transporter.

FIG. 7 is a graph of the total steviol glycosides relative to parent in the supernatant. Yeast strains with different overexpressed transporters were grown in microtiter plates. This figure reports the sum total of all steviol glycosides (measured in pmoles) that is detected in the supernatant after cells have been removed, relative to the parent strain. The parent strain does not contain an overexpressed transporter.

FIG. 8 shows the percent of all steviol glycosides produced located in the supernatant. Yeast strains with different overexpressed transporters were grown in microtiter plates. This figure reports the percent of all steviol glycosides produced by the cells (measured in pmoles) that is detected in the supernatant. The amount of total steviol glycosides measured in the supernatant is divided by the amount of total steviol glycosides measured in the whole cell broth to obtain the percent of total steviol glycosides in the supernatant.

FIG. 9 is a graph of the amount of Reb D+Reb M relative to parent in whole cell broth. Yeast strains expressing GFP-tagged and untagged versions of BPT1 and T4_Fungal_5 Transporter were grown in microtiter plates. The relative activities of the GFP-tagged and untagged versions of the transporters were compared. The data demonstrates that the GFP-tagged versions behaved similarly to the untagged versions of the transporters.

FIG. 10 is a set of photomicrographs of brightfield (A) and fluorescence (B) images of yeast expressing GFP-tagged BPT1.

FIG. 11 is a set of photomicrographs of brightfield (A) and fluorescence (B) images of yeast expressing GFP-tagged T4_Fungal_5 transporter.

FIG. 12 is a graph of the amount of Reb M relative to parent with wild type T4_Fungal_5 in whole cell broth. Yeast strains expressing transporters T4_Fungal_5 and its variants (Isolate_1-8) derived via error prone PCR and selection were grown in microtiter plates. This figure reports the Reb M titer (measured in pmoles) that is detected in whole cell broth (both cells and supernatant) of yeast strains expressing mutagenized T4_Fungal_5 transporter variants (Isolate_1-8) relative to unmutagenized T4_Fungal_5. The data demonstrates that expression of Isolates_1-8 resulted in improved Reb M production by yeast strains in comparison to T4_Fungal_5.

FIG. 13 is a graph of Reb M fraction of total steviol glycosides relative to parent with wild type T4_Fungal_5 in whole cell broth. Yeast strains expressing transporters T4_Fungal_5 and its variants (Isolate_1-8) derived via error prone PCR and selection were grown in microtiter plates. This figure reports the ratio of Reb M to the sum total of all steviol glycosides (measured in pmoles) that is detected in whole cell broth (both cells and supernatant) of yeast strains expressing mutagenized T4_Fungal_5 transporter variants (Isolate_1-8) relative to unmutagenized T4_Fungal_5. The data demonstrates that expression of Isolates_1-8 resulted in increased fraction of Reb M among all steviol glycosides in comparison to T4_Fungal_5 transporter. In other words, Isolates_1-8 display increased substrate preference for Reb M.

FIG. 14 is a graph of the amount of Reb M in whole cell broth and supernatant fraction produced by strains expressing either T4_Fungal_5 or Fungal_5_muA transporters. Yeast strains expressing T4_Fungal_5 or Fungal_5_muA under the control of PGAL3 (lower strength than PGAL1) were grown in microtiter plates. This figure reports the Reb M titer (measured in pmoles) that is detected in whole cell broth (both cells and supernatant) and supernatant fraction of yeast strains. The data confirms that Fungal_5_muA indeed confers improved performance when expressed in yeast strain: 30% more Reb M in whole cell broth and 40% more extracellular Reb M were produced by the strain with Fungal_5_muA than by the strain with the wild type T4_Fungal_5 when both transporters were expressed under lower promoter strength.

FIG. 15 is a graph of the amount of Reb M relative to parent with Fungal_5_muA in whole cell broth. Yeast strains expressing transporter Fungal_5_muA and eight of its variants where one, two, or three mutations were reverted to wild type T4_Fungal_5 sequence were grown in microtiter plates. This figure reports the Reb M titer (measured in pmoles) that is detected in whole cell broth (both cells and supernatant) of yeast strains expressing eight Fungal_5_muA variants relative to Fungal_5_muA. The data demonstrates the effect of different mutations on Reb M production, particularly interesting is the beneficial effect of E1320V reversion.

FIG. 16 is a graph of total steviol glycosides relative to parent with Fungal_5_muA in whole cell broth. Yeast strains expressing transporter Fungal_5_muA and eight of its variants where one, two, or three mutations were reverted to wild type T4_Fungal_5 sequence were grown in microtiter plates. This figure reports the sum total of all steviol glycosides (measured in pmoles) that is detected in whole cell broth (both cells and supernatant) of yeast strains expressing eight Fungal_5_muA variants relative to Fungal_5_muA. The data demonstrates the effect of different mutations on TSG production. Together with FIG. 15, it illustrates not only differences in activity but also substrate preference.

### 6. DETAILED DESCRIPTION OF THE EMBODIMENTS

### 6.1 Terminology

As used herein, the term “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); or (c) be naturally found in the host cell but positioned outside of its natural locus.

On the other hand, the term “native” or “endogenous” as used herein with reference to molecules, and in particular enzymes and nucleic acids, indicates molecules that are expressed in the organism in which they originated or are found in nature. It is understood that expression of native enzymes or polynucleotides may be modified in recombinant microorganisms.

As used herein, the term “heterologous nucleic acid expression cassette” refers to a nucleic acid sequence that comprises a coding sequence operably linked to one or more regulatory elements sufficient to expresses the coding sequence in a host cell. In an embodiment “ABC-transporter expression cassette” refers to a heterologous nucleic acid expression cassette in which the heterologous nucleic acid comprises the coding sequence for an ABC-transporter polypeptide. Non-limiting examples of regulatory elements include promoters, enhancers, silencers, terminators, and poly-A signals.

As used therein, the terms “ABC-transporter” and “ATP Binding Cassette Transporter” refer to a super-family of membrane associated polypeptides that couple adenosine triphosphate (ATP) hydrolysis to the translocation of various substrates across biological membranes.

As used herein, the term “CEN.PK.BPT1” refers to an ABC-transporter having
the following amino acid sequence (SEQ ID NO: 1):
MSSLEVVDGCPYGYRPYPDSGTNALNPCFISVISAWQAVFFLLIGSYQLWKLYKNNKVPPRFKNFPTLPSKINSRHLT
HLTNVCFQSTLIICELALVSQSSDRVYPFILKKALYLNLLFNLGISLPTQYLAYFKSTFSMGNQLFYYMFQILLQLFLILQR
YYHGSSNERLTVISGQTAMILEVLLLFNSVAIFIYDLCIFEPINELSEYYKKNGWYPPVHVLSYITFIWMNKLIVETYRNK
KIKDPNQLPLPPVDLNIKSISKEFKANWELEKWLNRNSLWRAIWKSFGRTISVAMLYETTSDLLSVVQPQFLRIFIDG
LNPETSSKYPPLNGVFIALTLFVISVVSVFLTNQFYIGIFEAGLGIRGSLASLVYQKSLRLTLAERNEKSTGDILNLMSVD
VLRIQRFFENAQTIIGAPIQIIVVLTSLYWLLGKAVIGGLVTMAIMMPINAFLSRKVKKLSKTQMKYKDMRIKTITELL
NAIKSIKLYAWEEPMMARLNHVRNDMELKNFRKIGIVSNLIYFAWNCVPLMVTCSTFGLFSLFSDSPLSPAIVFPSLS
LFNILNSAIYSVPSMINTIIETSVSMERLKSFLLSDEIDDSFIERIDPSADERALPAIEMNNITFLWKSKEVLTSSQSGDN
LRTDEESIIGSSQIALKNIDHFEAKRGDLVCVVGRVGAGKSTFLKAILGQLPCMSGSRDSIPPKLIIRSSSVAYCSQESW
IMNASVRENILFGHKFDQDYYDLTIKACQLLPDLKILPDGDETLVGEKGISLSGGQKARLSLARAVYSRADIYLLDDILS
AVDAEVSKNIIEYVLIGKTALLKNKTIILTTNTVSILKHSQMIYALENGEIVEQGNYEDVMNRKNNTSKLKKLLEEFDSP
IDNGNESDVQTEHRSESEVDEPLQLKVTESETEDEVVTESELELIKANSRRASLATLRPRPFVGAQLDSVKKTAQKAE
KTEVGRVKTKIYLAYIKACGVLGVVLFFLFMILTRVFDLAENFWLKYWSESNEKNGSNERVWMFVGVYSLIGVASA
AFNNLRSIMMLLYCSIRGSKKLHESMAKSVIRSPMTFFETTPVGRIINRFSSDMDAVDSNLQYIFSFFFKSILTYLVTVI
LVGYNMPWFLVFNMFLVVIYIYYQTFYIVLSRELKRLISISYSPIMSLMSESLNGYSIIDAYDHFERFIYLNYEKIQYNVD
FVFNFRSTNRWLSVRLQTIGATIVLATAILALATMNTKRQLSSGMVGLLMSYSLEVTGSLTWIVRTTVTIETNIVSVE
RIVEYCELPPEAQSINPEKRPDENWPSKGGIEFKNYSTKYRENLDPVLNNINVKIEPCEKVGIVGRTGAGKSTLSLALF
RILEPTEGKIIIDGIDISDIGLFDLRSHLAIIPQDAQAFEGTVKTNLDPFNRYSEDELKRAVEQAHLKPHLEKMLHSKPR
GDDSNEEDGNVNDILDVKINENGSNLSVGQRQLLCLARALLNRSKILVLDEATASVDMETDKIIQDTIRREFKDRTIL
TIAHRIDTVLDSDKIIVLDQGSVREFDSPSKLLSDKTSIFYSLCEKGGYLK*;
and
encoded by the following nucleic acid sequence (SEQ ID NO: 20):
ATGTCTTCACTAGAAGTGGTAGATGGGTGCCCCTATGGATACCGACCATATCCAGATAGTGGCACAAATGCAT
TAAATCCATGTTTTATATCAGTAATATCCGCCTGGCAAGCCGTCTTTTTCCTATTGATTGGTAGCTATCAATTGT
GGAAACTTTATAAGAACAATAAAGTACCACCCAGATTTAAGAACTTTCCTACATTACCAAGTAAAATCAACAGT
CGACATCTAACGCATTTGACCAATGTTTGCTTTCAGTCCACGCTTATAATTTGTGAACTGGCCTTGGTATCCCAA
TCTAGCGATAGGGTTTATCCATTTATACTAAAGAAGGCTCTGTACTTGAATCTCCTTTTCAATTTGGGTATTTCT
CTCCCTACTCAATACTTAGCTTATTTTAAAAGTACATTTTCAATGGGCAACCAGCTTTTCTATTACATGTTTCAAA
TTCTTCTACAGCTCTTCTTGATATTGCAGAGGTACTATCATGGTTCTAGTAACGAAAGGCTTACTGTTATTAGCG
GACAAACTGCTATGATTTTAGAAGTGCTCCTTCTTTTCAATTCTGTGGCAATTTTTATTTATGATCTATGCATTTT
TGAGCCAATTAACGAATTATCTGAATACTACAAGAAAAATGGGTGGTATCCCCCCGTTCATGTACTATCCTATA
TTACATTTATCTGGATGAACAAACTGATTGTGGAAACTTACCGTAACAAGAAAATCAAAGATCCTAACCAGTTA
CCATTGCCGCCAGTAGATCTGAATATTAAGTCGATAAGTAAGGAATTTAAGGCTAACTGGGAATTGGAAAAAT
GGTTGAATAGAAATTCTCTTTGGAGGGCCATTTGGAAGTCATTTGGTAGGACTATTTCTGTGGCTATGCTGTAT
GAAACGACATCTGATTTACTTTCTGTAGTACAGCCCCAGTTTCTACGGATATTCATAGATGGTTTGAACCCGGA
AACATCTTCTAAATATCCTCCTTTAAATGGTGTATTTATTGCTCTAACCCTTTTCGTAATCAGCGTGGTTTCTGTG
TTCCTCACCAATCAATTTTATATTGGAATTTTTGAGGCTGGTTTGGGGATAAGAGGCTCTTTAGCTTCTTTAGTG
TATCAGAAGTCCTTAAGATTGACGCTAGCAGAGCGTAACGAAAAATCTACTGGTGACATCTTAAATTTGATGT
CTGTGGATGTGTTAAGGATCCAGCGGTTTTTCGAAAATGCCCAAACCATTATTGGCGCTCCTATTCAGATTATT
GTTGTATTAACTTCCCTGTACTGGTTGCTAGGAAAGGCTGTTATTGGAGGGTTGGTTACTATGGCTATTATGAT
GCCTATCAATGCCTTCTTATCTAGAAAGGTAAAAAAGCTATCAAAAACTCAAATGAAGTATAAGGACATGAGA
ATCAAGACTATTACAGAGCTTTTGAATGCTATAAAATCTATTAAATTATACGCCTGGGAGGAACCTATGATGGC
AAGATTGAATCATGTTCGTAATGATATGGAGTTGAAAAATTTTCGGAAAATTGGTATAGTGAGCAATCTGATA
TATTTTGCGTGGAATTGTGTACCTTTAATGGTGACATGTTCCACATTTGGCTTATTTTCTTTATTTAGTGATTCTC
CGTTATCTCCTGCCATTGTCTTCCCTTCATTATCTTTATTTAATATTTTGAACAGTGCCATCTATTCCGTTCCATCC
ATGATAAATACCATTATAGAGACAAGCGTTTCTATGGAAAGATTAAAGTCATTCCTACTTAGTGACGAAATTG
ATGATTCGTTCATCGAACGTATTGATCCTTCAGCGGATGAAAGAGCGTTACCTGCTATAGAGATGAATAATATT
ACATTTTTATGGAAATCAAAAGAAGTATTAACATCTAGCCAATCTGGAGATAATTTGAGGACAGATGAAGAGT
CTATTATCGGATCTTCTCAAATTGCGTTGAAGAATATCGATCATTTTGAAGCAAAAAGGGGTGATTTAGTTTGT
GTTGTTGGTCGGGTAGGAGCTGGTAAATCAACATTTTTGAAGGCAATTCTTGGTCAACTTCCTTGCATGAGTG
GTTCTAGGGACTCGATACCACCTAAACTGATCATTAGATCATCGTCTGTAGCCTACTGTTCACAAGAATCCTGG
ATAATGAACGCATCTGTAAGAGAAAACATTCTATTTGGTCACAAGTTCGACCAAGATTATTATGACCTCACTAT
TAAAGCATGTCAATTGCTACCCGATTTGAAAATACTACCAGATGGTGATGAAACTTTGGTAGGTGAAAAGGGC
ATTTCCCTATCAGGCGGTCAGAAGGCCCGTCTTTCATTAGCCAGAGCGGTGTACTCGAGAGCAGATATTTATTT
GTTGGATGACATTTTATCTGCTGTTGATGCAGAAGTTAGTAAAAATATTATTGAATATGTTTTGATCGGAAAGA
CGGCTTTATTAAAAAATAAAACAATTATTTTAACTACCAATACTGTATCAATTTTAAAACATTCGCAGATGATAT
ATGCGCTAGAAAACGGTGAAATTGTTGAACAAGGGAATTATGAGGATGTAATGAACCGTAAGAACAATACTT
CAAAACTGAAAAAATTACTAGAGGAATTTGATTCTCCGATTGATAATGGAAATGAAAGCGATGTCCAAACTGA
ACACCGATCCGAAAGTGAAGTGGATGAACCTCTGCAGCTTAAAGTAACTGAATCAGAAACTGAGGATGAGGT
TGTTACTGAGAGTGAATTAGAACTAATCAAAGCCAATTCTAGAAGAGCTTCTCTAGCTACGCTAAGACCTAGA
CCCTTTGTGGGAGCACAATTGGATTCCGTGAAGAAAACGGCGCAAAAGGCCGAGAAGACAGAGGTGGGAAG
AGTCAAAACAAAGATTTATCTTGCGTATATTAAGGCTTGTGGAGTTTTAGGTGTTGTTTTATTTTTCTTGTTTAT
GATATTAACAAGGGTTTTCGACTTAGCAGAGAATTTTTGGTTAAAGTACTGGTCAGAATCTAATGAAAAAAAT
GGTTCAAATGAAAGGGTTTGGATGTTTGTTGGTGTGTATTCCTTAATCGGAGTAGCATCGGCCGCATTCAATA
ATTTACGGAGTATTATGATGCTACTGTATTGTTCTATTAGGGGTTCTAAGAAACTGCATGAAAGCATGGCCAA
ATCTGTAATTAGAAGTCCTATGACTTTCTTTGAGACTACACCAGTTGGAAGGATCATAAACAGGTTCTCATCTG
ATATGGATGCAGTGGACAGTAATCTACAGTACATTTTCTCCTTTTTTTTCAAATCAATACTAACCTATTTGGTTA
CTGTTATATTAGTCGGGTACAATATGCCATGGTTTTTAGTGTTCAATATGTTTTTGGTGGTTATCTATATTTACT
ATCAAACATTTTACATTGTGCTATCTAGGGAGCTAAAAAGATTGATCAGTATATCTTACTCTCCGATTATGTCCT
TAATGAGTGAGAGCTTGAACGGTTATTCTATTATTGATGCATACGATCATTTTGAGAGATTCATCTATCTAAAT
TATGAAAAAATCCAATACAACGTTGATTTTGTCTTCAACTTTAGATCAACGAATAGATGGTTATCCGTGAGATT
GCAAACTATTGGTGCTACAATTGTTTTGGCTACTGCAATCTTAGCACTAGCAACAATGAATACTAAAAGGCAAC
TAAGTTCGGGTATGGTTGGTCTACTAATGAGCTATTCATTAGAGGTTACAGGTTCATTGACTTGGATTGTAAG
GACAACTGTGACGATTGAAACCAACATTGTATCAGTGGAGAGAATTGTTGAGTACTGCGAATTACCACCTGAA
GCACAGTCCATTAACCCTGAAAAGAGGCCAGATGAAAATTGGCCATCAAAGGGTGGTATTGAATTCAAAAAC
TATTCCACAAAATACAGAGAAAATTTGGATCCAGTGCTGAATAATATTAACGTGAAGATTGAGCCATGTGAAA
AGGTTGGGATTGTTGGCAGAACAGGTGCAGGGAAGTCTACACTGAGCCTGGCATTATTTAGAATACTAGAAC
CTACCGAAGGTAAAATTATTATTGACGGCATTGATATATCCGACATAGGTCTGTTCGATTTAAGAAGCCATTTG
GCAATTATTCCTCAGGATGCACAAGCTTTTGAAGGTACAGTAAAGACCAATTTGGACCCTTTCAATCGTTATTC
AGAAGATGAACTTAAAAGGGCTGTTGAGCAGGCACATTTAAAGCCTCATCTGGAAAAAATGCTGCACAGTAA
ACCAAGAGGTGATGATTCTAATGAAGAGGATGGCAATGTTAATGATATTCTGGATGTCAAGATTAATGAGAA
CGGTAGTAACTTGTCAGTGGGGCAAAGACAACTACTATGTTTGGCAAGAGCGCTGCTAAACCGTTCCAAAATA
TTGGTCCTTGATGAAGCAACGGCTTCTGTGGATATGGAAACCGATAAAATTATCCAAGACACTATAAGAAGAG
AATTTAAGGACCGTACCATCTTAACAATTGCACATCGTATCGACACTGTATTGGACAGTGATAAGATAATTGTT
CTTGACCAGGGTAGTGTGAGGGAATTCGATTCACCCTCGAAATTGTTATCCGATAAAACGTCTATTTTTTACAG
TCTTTGTGAGAAAGGTGGGTATTTGAAATAA.
As used herein, the term “T4_Fungal_1” refers to an ABC-transporter having the
following amino acid sequence (SEQ ID NO: 2):
MSLELSNSTLCDSYWAVDDFTACGRQLVESWVSVPLVLSALVVAFNLLRNSLASEKTDPYSKLDAEQQPLLQNGHA
LYTSSIESDNTDIFQRHFDIALLKPVKDDGKPIGVVRIVYRDTAEKLKVALEEILLISQTVLAFLALSRLEDISESRFLLVKY
INFSLWLYLTVITSARLLNVTKGFSANRVDLWYHCAILYNLQWFNSVMLFRSALLHHVSGTYGYWFYVTQFVINTLL
CLTNGLEKLSDKPAIVYEEEGVIPSPETTSSLIDIMTYGYLDKMVFSSYWKPITMEEVWGLRYDDYSHDVLIRFHKLKS
SIRFTLRLFLQFKKELALQTLCTCIEALLIFVPPLCLKKILEYIESPHTKSRSMAWFYVLIMFGSGVIACSFSGRGLFLGRRI
CTRMRSILIGEIYSKALRRRLGSTDKEKTTEEEDDKSAKSKKEDEPSNKELGGIINLMAVDAFKVSEIGGYLHYFPNSFV
MAAVAIYMLYKLLGWSSLIGTATLIAILPINYMLVEKLSKYQKQMLLVTDKRIQKTNEAFQNIRIIKYFAWEDKFADTI
MKIREEELGYLVGRCVVWALLIFLWLVVPTIVTLITFYAYTVIQGNPLTSPIAFTALSLFTLLRGPLDALADMLSMVM
QCKVSLDRVEDFLNEPETTKYQQLSAPRGPNSPLIGFENATFYWSKNSKAEFALKDLNIDFKVGKLNVVIGPTGSGK
SSLLLALLGEMDLDKGNVFLPGAIPRDDLTPNPVTGLMESVAYCSQTAWLLNATVKDNIIFASPFNQERYDAVIHAC
GLTRDLSILEAGDETEIGEKGITLSGGQKQRVSLARALYSSASYLLLDDCLSAVDSHTAVHIYDYCINGELMKGRTCILV
SHNVSLTVKEADFVVMMDNGRIKAQGSVDELMQEGLLNEEVVKSVMQSRSASTANLAALDDNSPISSEAIAEGLA
KKTQKPEQSKKSKLIEDETKSDGSVKPEIYYAYFRYFGNPALWIMIAFLFIGSQSVNVYQSYWLRRWSAIEDKRDLSA
FSNSNDMTLFLFPTFHSINWHRPLVNYALQPFGLAVEERSTMYYITIYTLIGLAFATLGSSRVILTFIGGLNVSRKIFKD
LLDKLLHAKLRFFDQTPIGRIMNRFSKDIEAIDQELALYAEEFVTYLISCLSTLVVVCAVTPAFLVAGVLILLVYYGVGVL
YLELSRDLKRFESITKSPIHQHFSETLVGMTTIRAYGDERRFLKQNFEKIDVNNRPFWYVWVNNRWLAYRSDMIGA
FIIFFAAAFAVAYSDKIDAGLAGISLSFSVSFRYTAVWVVRMYAYVEMSMNSVERVQEYIEQTPQEPPKYLPQDPVN
SWPSNGVIDVQNICIRYSPELPRVIDNVSFHVNAGEKIGVVGRTGAGKSTIITSFFRFVDLESGSIKIDGLDISKIGLKPL
RKGLTIIPQDPTLFSGTIRSNLDIFGEYGDLQMFEALRRVNLISVDDYQRIVDGNGAAVADETAQARGDNVNKFLDL
DSTVSEGGGNLSQGERQLLCLARSILKMPKILMLDEATASIDYESDAKIQATIREEFSSSTVLTIAHRLKTIIDYDKILLLD
HGKVKEYDHPYKLITNKKSDFRKMCQDTGEFDDLVNLAKQAYRK*;
and
encoded by the following nucleic acid sequence (SEQ ID NO: 21):
ATGTCTTCACTAGAAGTGGTAGATGGGTGCCCCTATGGATACCGACCATATCCAGATAGTGGCACAAATGCAT
TAAATCCATGTTTTATATCAGTAATATCCGCCTGGCAAGCCGTCTTTTTCCTATTGATTGGTAGCTATCAATTGT
GGAAACTTTATAAGAACAATAAAGTACCACCCAGATTTAAGAACTTTCCTACATTACCAAGTAAAATCAACAGT
CGACATCTAACGCATTTGACCAATGTTTGCTTTCAGTCCACGCTTATAATTTGTGAACTGGCCTTGGTATCCCAA
TCTAGCGATAGGGTTTATCCATTTATACTAAAGAAGGCTCTGTACTTGAATCTCCTTTTCAATTTGGGTATTTCT
CTCCCTACTCAATACTTAGCTTATTTTAAAAGTACATTTTCAATGGGCAACCAGCTTTTCTATTACATGTTTCAAA
TTCTTCTACAGCTCTTCTTGATATTGCAGAGGTACTATCATGGTTCTAGTAACGAAAGGCTTACTGTTATTAGCG
GACAAACTGCTATGATTTTAGAAGTGCTCCTTCTTTTCAATTCTGTGGCAATTTTTATTTATGATCTATGCATTTT
TGAGCCAATTAACGAATTATCTGAATACTACAAGAAAAATGGGTGGTATCCCCCCGTTCATGTACTATCCTATA
TTACATTTATCTGGATGAACAAACTGATTGTGGAAACTTACCGTAACAAGAAAATCAAAGATCCTAACCAGTTA
CCATTGCCGCCAGTAGATCTGAATATTAAGTCGATAAGTAAGGAATTTAAGGCTAACTGGGAATTGGAAAAAT
GGTTGAATAGAAATTCTCTTTGGAGGGCCATTTGGAAGTCATTTGGTAGGACTATTTCTGTGGCTATGCTGTAT
GAAACGACATCTGATTTACTTTCTGTAGTACAGCCCCAGTTTCTACGGATATTCATAGATGGTTTGAACCCGGA
AACATCTTCTAAATATCCTCCTTTAAATGGTGTATTTATTGCTCTAACCCTTTTCGTAATCAGCGTGGTTTCTGTG
TTCCTCACCAATCAATTTTATATTGGAATTTTTGAGGCTGGTTTGGGGATAAGAGGCTCTTTAGCTTCTTTAGTG
TATCAGAAGTCCTTAAGATTGACGCTAGCAGAGCGTAACGAAAAATCTACTGGTGACATCTTAAATTTGATGT
CTGTGGATGTGTTAAGGATCCAGCGGTTTTTCGAAAATGCCCAAACCATTATTGGCGCTCCTATTCAGATTATT
GTTGTATTAACTTCCCTGTACTGGTTGCTAGGAAAGGCTGTTATTGGAGGGTTGGTTACTATGGCTATTATGAT
GCCTATCAATGCCTTCTTATCTAGAAAGGTAAAAAAGCTATCAAAAACTCAAATGAAGTATAAGGACATGAGA
ATCAAGACTATTACAGAGCTTTTGAATGCTATAAAATCTATTAAATTATACGCCTGGGAGGAACCTATGATGGC
AAGATTGAATCATGTTCGTAATGATATGGAGTTGAAAAATTTTCGGAAAATTGGTATAGTGAGCAATCTGATA
TATTTTGCGTGGAATTGTGTACCTTTAATGGTGACATGTTCCACATTTGGCTTATTTTCTTTATTTAGTGATTCTC
CGTTATCTCCTGCCATTGTCTTCCCTTCATTATCTTTATTTAATATTTTGAACAGTGCCATCTATTCCGTTCCATCC
ATGATAAATACCATTATAGAGACAAGCGTTTCTATGGAAAGATTAAAGTCATTCCTACTTAGTGACGAAATTG
ATGATTCGTTCATCGAACGTATTGATCCTTCAGCGGATGAAAGAGCGTTACCTGCTATAGAGATGAATAATATT
ACATTTTTATGGAAATCAAAAGAAGTATTAACATCTAGCCAATCTGGAGATAATTTGAGGACAGATGAAGAGT
CTATTATCGGATCTTCTCAAATTGCGTTGAAGAATATCGATCATTTTGAAGCAAAAAGGGGTGATTTAGTTTGT
GTTGTTGGTCGGGTAGGAGCTGGTAAATCAACATTTTTGAAGGCAATTCTTGGTCAACTTCCTTGCATGAGTG
GTTCTAGGGACTCGATACCACCTAAACTGATCATTAGATCATCGTCTGTAGCCTACTGTTCACAAGAATCCTGG
ATAATGAACGCATCTGTAAGAGAAAACATTCTATTTGGTCACAAGTTCGACCAAGATTATTATGACCTCACTAT
TAAAGCATGTCAATTGCTACCCGATTTGAAAATACTACCAGATGGTGATGAAACTTTGGTAGGTGAAAAGGGC
ATTTCCCTATCAGGCGGTCAGAAGGCCCGTCTTTCATTAGCCAGAGCGGTGTACTCGAGAGCAGATATTTATTT
GTTGGATGACATTTTATCTGCTGTTGATGCAGAAGTTAGTAAAAATATTATTGAATATGTTTTGATCGGAAAGA
CGGCTTTATTAAAAAATAAAACAATTATTTTAACTACCAATACTGTATCAATTTTAAAACATTCGCAGATGATAT
ATGCGCTAGAAAACGGTGAAATTGTTGAACAAGGGAATTATGAGGATGTAATGAACCGTAAGAACAATACTT
CAAAACTGAAAAAATTACTAGAGGAATTTGATTCTCCGATTGATAATGGAAATGAAAGCGATGTCCAAACTGA
ACACCGATCCGAAAGTGAAGTGGATGAACCTCTGCAGCTTAAAGTAACTGAATCAGAAACTGAGGATGAGGT
TGTTACTGAGAGTGAATTAGAACTAATCAAAGCCAATTCTAGAAGAGCTTCTCTAGCTACGCTAAGACCTAGA
CCCTTTGTGGGAGCACAATTGGATTCCGTGAAGAAAACGGCGCAAAAGGCCGAGAAGACAGAGGTGGGAAG
AGTCAAAACAAAGATTTATCTTGCGTATATTAAGGCTTGTGGAGTTTTAGGTGTTGTTTTATTTTTCTTGTTTAT
GATATTAACAAGGGTTTTCGACTTAGCAGAGAATTTTTGGTTAAAGTACTGGTCAGAATCTAATGAAAAAAAT
GGTTCAAATGAAAGGGTTTGGATGTTTGTTGGTGTGTATTCCTTAATCGGAGTAGCATCGGCCGCATTCAATA
ATTTACGGAGTATTATGATGCTACTGTATTGTTCTATTAGGGGTTCTAAGAAACTGCATGAAAGCATGGCCAA
ATCTGTAATTAGAAGTCCTATGACTTTCTTTGAGACTACACCAGTTGGAAGGATCATAAACAGGTTCTCATCTG
ATATGGATGCAGTGGACAGTAATCTACAGTACATTTTCTCCTTTTTTTTCAAATCAATACTAACCTATTTGGTTA
CTGTTATATTAGTCGGGTACAATATGCCATGGTTTTTAGTGTTCAATATGTTTTTGGTGGTTATCTATATTTACT
ATCAAACATTTTACATTGTGCTATCTAGGGAGCTAAAAAGATTGATCAGTATATCTTACTCTCCGATTATGTCCT
TAATGAGTGAGAGCTTGAACGGTTATTCTATTATTGATGCATACGATCATTTTGAGAGATTCATCTATCTAAAT
TATGAAAAAATCCAATACAACGTTGATTTTGTCTTCAACTTTAGATCAACGAATAGATGGTTATCCGTGAGATT
GCAAACTATTGGTGCTACAATTGTTTTGGCTACTGCAATCTTAGCACTAGCAACAATGAATACTAAAAGGCAAC
TAAGTTCGGGTATGGTTGGTCTACTAATGAGCTATTCATTAGAGGTTACAGGTTCATTGACTTGGATTGTAAG
GACAACTGTGACGATTGAAACCAACATTGTATCAGTGGAGAGAATTGTTGAGTACTGCGAATTACCACCTGAA
GCACAGTCCATTAACCCTGAAAAGAGGCCAGATGAAAATTGGCCATCAAAGGGTGGTATTGAATTCAAAAAC
TATTCCACAAAATACAGAGAAAATTTGGATCCAGTGCTGAATAATATTAACGTGAAGATTGAGCCATGTGAAA
AGGTTGGGATTGTTGGCAGAACAGGTGCAGGGAAGTCTACACTGAGCCTGGCATTATTTAGAATACTAGAAC
CTACCGAAGGTAAAATTATTATTGACGGCATTGATATATCCGACATAGGTCTGTTCGATTTAAGAAGCCATTTG
GCAATTATTCCTCAGGATGCACAAGCTTTTGAAGGTACAGTAAAGACCAATTTGGACCCTTTCAATCGTTATTC
AGAAGATGAACTTAAAAGGGCTGTTGAGCAGGCACATTTAAAGCCTCATCTGGAAAAAATGCTGCACAGTAA
ACCAAGAGGTGATGATTCTAATGAAGAGGATGGCAATGTTAATGATATTCTGGATGTCAAGATTAATGAGAA
CGGTAGTAACTTGTCAGTGGGGCAAAGACAACTACTATGTTTGGCAAGAGCGCTGCTAAACCGTTCCAAAATA
TTGGTCCTTGATGAAGCAACGGCTTCTGTGGATATGGAAACCGATAAAATTATCCAAGACACTATAAGAAGAG
AATTTAAGGACCGTACCATCTTAACAATTGCACATCGTATCGACACTGTATTGGACAGTGATAAGATAATTGTT
CTTGACCAGGGTAGTGTGAGGGAATTCGATTCACCCTCGAAATTGTTATCCGATAAAACGTCTATTTTTTACAG
TCTTTGTGAGAAAGGTGGGTATTTGAAATAA.
As used herein, the term “T4_Fungal_10” refers to an ABC-transporter having the
following amino acid sequence (SEQ ID NO: 3):
MGQSERAALIAFASRNTTECWLCRDKEGFGPISYYGDFTVCFIDGVLLNFAALFMLIFGTYQVVKLSKKEHPGIKYRR
DWLLFSRITLVGCFLLFTSMAAYYSSEKHESIALTSQYTLTLMSIFVALMLHWVEYHRSRISNGIVLFYWLFETLFQGS
KWVNFSIRHAYNLNHEWPVSYSVYILTIFQTISAFMILILEAGFEKPLPSYQRVIESYSKQKRNPVDNSHIFQRLSFSW
MTELMKTGYKKYLTEQDLYKLPKSFGAKEISHKFSERWQYQLKHKANPSLAWALLSTFGGKILLGGIFKVAYDILQFT
QPQLLRILIKFVSDYTSTPEPQLPLVRGVMLSIAMFVVSVVQTSILHQYFLNAFDTGMHIKSGMTSVIYQKALVLSSE
ASASSSTGDIVNLMSVDVQRLQDLTQWGQIIWSGPFQIILCLVSLYKLLGPCMWVGVIIMIIMIPINSVIVRIQKKLQ
KIQMKNKDERTRVTSEILNNIKSLKVYGWEIPYKAKLDHVRNDKELKNLKKMGCTLALASFQFNIVPFLVSCSTFAVF
VFTEDRPLSTDLVFPALTLFNLLSFPLAVVPNAISSFIEASVSVNRLYAFLTNEELQTDAVHREPKVNNIGDEGVKVSD
ATFLWQRKPEYKVALKNINFSAKKGELTCIVGKVGSGKSALIQSLLGDLIRVKGYAAVHGSVAYVSQVAWIMNGTV
KDNIIFGHKYDPEFYELTIKACALAIDLSMLPDGDQTLVGEKGISLSGGQKARLSLARAVYARADTYLLDDPLAAVDE
HVAKHLIEHVLGPHGLLHSKTKVLATNKISVLSIADSITLMENGEIIQQGTYEETNNTTDSPLSKLISEFGKKGKATPSQ
STTSLTKLATSDLGSSSDSKVSDVSIDVSQLDTENLTEAEELKSLRRASMATLGSIGFDDDENIARREHREQGKVKWD
IYMEYARACNPRSVCVFLFFIVLSMLLSVLGNFWLKHWSEVNTGEGYNPHAARYLLIYFALGVGSALATLIQTIVLW
VFCTIHGSRYLHDAMATSVLKAPMSFFETTPIGRILNRFSNDIYKVDEVLGRTFSQFFANVVKVSFTIIVICMATWQFI
FIILPLSVLYIYYQQYYLRTSRELRRLDSVTRSPIYAHFQETLGGLTTIRGYSQQTRFVHINQTRVDNNMSAFYPSVNA
NRWLAFRLEFIGSIIILGSSMLAVIRLGNGTLTAGMIGLSLSFALQITQSLNWIVRMTVEVETNIVSVERIKEYAELKSE
APYIIEDHRPPASWPEKGDVKFVNYSTRYRPELELILKDINLHILPKEKIGIVGRTGAGKSSLTLALFRIIEAASGHIIIDGI
PIDSIGLADLRHRLSIIPQDSQIFEGTIRENIDPSKQYTDEQIWDALELSHLKNHVKNMGPDGLETMLSEGGGNLSVG
QRQLMCLARALLISSKILVLDEATAAVDVETDQLIQKTIREAFKERTILTIAHRINTIMDSDRIIVLDKGRVTEFDTPANL
LNKKDSIFYSLCVEAGLAE*;
and
encoded by the following nucleic acid sequence (SEQ ID NO: 22):
ATGTCTTCACTAGAAGTGGTAGATGGGTGCCCCTATGGATACCGACCATATCCAGATAGTGGCACAAATGCAT
TAAATCCATGTTTTATATCAGTAATATCCGCCTGGCAAGCCGTCTTTTTCCTATTGATTGGTAGCTATCAATTGT
GGAAACTTTATAAGAACAATAAAGTACCACCCAGATTTAAGAACTTTCCTACATTACCAAGTAAAATCAACAGT
CGACATCTAACGCATTTGACCAATGTTTGCTTTCAGTCCACGCTTATAATTTGTGAACTGGCCTTGGTATCCCAA
TCTAGCGATAGGGTTTATCCATTTATACTAAAGAAGGCTCTGTACTTGAATCTCCTTTTCAATTTGGGTATTTCT
CTCCCTACTCAATACTTAGCTTATTTTAAAAGTACATTTTCAATGGGCAACCAGCTTTTCTATTACATGTTTCAAA
TTCTTCTACAGCTCTTCTTGATATTGCAGAGGTACTATCATGGTTCTAGTAACGAAAGGCTTACTGTTATTAGCG
GACAAACTGCTATGATTTTAGAAGTGCTCCTTCTTTTCAATTCTGTGGCAATTTTTATTTATGATCTATGCATTTT
TGAGCCAATTAACGAATTATCTGAATACTACAAGAAAAATGGGTGGTATCCCCCCGTTCATGTACTATCCTATA
TTACATTTATCTGGATGAACAAACTGATTGTGGAAACTTACCGTAACAAGAAAATCAAAGATCCTAACCAGTTA
CCATTGCCGCCAGTAGATCTGAATATTAAGTCGATAAGTAAGGAATTTAAGGCTAACTGGGAATTGGAAAAAT
GGTTGAATAGAAATTCTCTTTGGAGGGCCATTTGGAAGTCATTTGGTAGGACTATTTCTGTGGCTATGCTGTAT
GAAACGACATCTGATTTACTTTCTGTAGTACAGCCCCAGTTTCTACGGATATTCATAGATGGTTTGAACCCGGA
AACATCTTCTAAATATCCTCCTTTAAATGGTGTATTTATTGCTCTAACCCTTTTCGTAATCAGCGTGGTTTCTGTG
TTCCTCACCAATCAATTTTATATTGGAATTTTTGAGGCTGGTTTGGGGATAAGAGGCTCTTTAGCTTCTTTAGTG
TATCAGAAGTCCTTAAGATTGACGCTAGCAGAGCGTAACGAAAAATCTACTGGTGACATCTTAAATTTGATGT
CTGTGGATGTGTTAAGGATCCAGCGGTTTTTCGAAAATGCCCAAACCATTATTGGCGCTCCTATTCAGATTATT
GTTGTATTAACTTCCCTGTACTGGTTGCTAGGAAAGGCTGTTATTGGAGGGTTGGTTACTATGGCTATTATGAT
GCCTATCAATGCCTTCTTATCTAGAAAGGTAAAAAAGCTATCAAAAACTCAAATGAAGTATAAGGACATGAGA
ATCAAGACTATTACAGAGCTTTTGAATGCTATAAAATCTATTAAATTATACGCCTGGGAGGAACCTATGATGGC
AAGATTGAATCATGTTCGTAATGATATGGAGTTGAAAAATTTTCGGAAAATTGGTATAGTGAGCAATCTGATA
TATTTTGCGTGGAATTGTGTACCTTTAATGGTGACATGTTCCACATTTGGCTTATTTTCTTTATTTAGTGATTCTC
CGTTATCTCCTGCCATTGTCTTCCCTTCATTATCTTTATTTAATATTTTGAACAGTGCCATCTATTCCGTTCCATCC
ATGATAAATACCATTATAGAGACAAGCGTTTCTATGGAAAGATTAAAGTCATTCCTACTTAGTGACGAAATTG
ATGATTCGTTCATCGAACGTATTGATCCTTCAGCGGATGAAAGAGCGTTACCTGCTATAGAGATGAATAATATT
ACATTTTTATGGAAATCAAAAGAAGTATTAACATCTAGCCAATCTGGAGATAATTTGAGGACAGATGAAGAGT
CTATTATCGGATCTTCTCAAATTGCGTTGAAGAATATCGATCATTTTGAAGCAAAAAGGGGTGATTTAGTTTGT
GTTGTTGGTCGGGTAGGAGCTGGTAAATCAACATTTTTGAAGGCAATTCTTGGTCAACTTCCTTGCATGAGTG
GTTCTAGGGACTCGATACCACCTAAACTGATCATTAGATCATCGTCTGTAGCCTACTGTTCACAAGAATCCTGG
ATAATGAACGCATCTGTAAGAGAAAACATTCTATTTGGTCACAAGTTCGACCAAGATTATTATGACCTCACTAT
TAAAGCATGTCAATTGCTACCCGATTTGAAAATACTACCAGATGGTGATGAAACTTTGGTAGGTGAAAAGGGC
ATTTCCCTATCAGGCGGTCAGAAGGCCCGTCTTTCATTAGCCAGAGCGGTGTACTCGAGAGCAGATATTTATTT
GTTGGATGACATTTTATCTGCTGTTGATGCAGAAGTTAGTAAAAATATTATTGAATATGTTTTGATCGGAAAGA
CGGCTTTATTAAAAAATAAAACAATTATTTTAACTACCAATACTGTATCAATTTTAAAACATTCGCAGATGATAT
ATGCGCTAGAAAACGGTGAAATTGTTGAACAAGGGAATTATGAGGATGTAATGAACCGTAAGAACAATACTT
CAAAACTGAAAAAATTACTAGAGGAATTTGATTCTCCGATTGATAATGGAAATGAAAGCGATGTCCAAACTGA
ACACCGATCCGAAAGTGAAGTGGATGAACCTCTGCAGCTTAAAGTAACTGAATCAGAAACTGAGGATGAGGT
TGTTACTGAGAGTGAATTAGAACTAATCAAAGCCAATTCTAGAAGAGCTTCTCTAGCTACGCTAAGACCTAGA
CCCTTTGTGGGAGCACAATTGGATTCCGTGAAGAAAACGGCGCAAAAGGCCGAGAAGACAGAGGTGGGAAG
AGTCAAAACAAAGATTTATCTTGCGTATATTAAGGCTTGTGGAGTTTTAGGTGTTGTTTTATTTTTCTTGTTTAT
GATATTAACAAGGGTTTTCGACTTAGCAGAGAATTTTTGGTTAAAGTACTGGTCAGAATCTAATGAAAAAAAT
GGTTCAAATGAAAGGGTTTGGATGTTTGTTGGTGTGTATTCCTTAATCGGAGTAGCATCGGCCGCATTCAATA
ATTTACGGAGTATTATGATGCTACTGTATTGTTCTATTAGGGGTTCTAAGAAACTGCATGAAAGCATGGCCAA
ATCTGTAATTAGAAGTCCTATGACTTTCTTTGAGACTACACCAGTTGGAAGGATCATAAACAGGTTCTCATCTG
ATATGGATGCAGTGGACAGTAATCTACAGTACATTTTCTCCTTTTTTTTCAAATCAATACTAACCTATTTGGTTA
CTGTTATATTAGTCGGGTACAATATGCCATGGTTTTTAGTGTTCAATATGTTTTTGGTGGTTATCTATATTTACT
ATCAAACATTTTACATTGTGCTATCTAGGGAGCTAAAAAGATTGATCAGTATATCTTACTCTCCGATTATGTCCT
TAATGAGTGAGAGCTTGAACGGTTATTCTATTATTGATGCATACGATCATTTTGAGAGATTCATCTATCTAAAT
TATGAAAAAATCCAATACAACGTTGATTTTGTCTTCAACTTTAGATCAACGAATAGATGGTTATCCGTGAGATT
GCAAACTATTGGTGCTACAATTGTTTTGGCTACTGCAATCTTAGCACTAGCAACAATGAATACTAAAAGGCAAC
TAAGTTCGGGTATGGTTGGTCTACTAATGAGCTATTCATTAGAGGTTACAGGTTCATTGACTTGGATTGTAAG
GACAACTGTGACGATTGAAACCAACATTGTATCAGTGGAGAGAATTGTTGAGTACTGCGAATTACCACCTGAA
GCACAGTCCATTAACCCTGAAAAGAGGCCAGATGAAAATTGGCCATCAAAGGGTGGTATTGAATTCAAAAAC
TATTCCACAAAATACAGAGAAAATTTGGATCCAGTGCTGAATAATATTAACGTGAAGATTGAGCCATGTGAAA
AGGTTGGGATTGTTGGCAGAACAGGTGCAGGGAAGTCTACACTGAGCCTGGCATTATTTAGAATACTAGAAC
CTACCGAAGGTAAAATTATTATTGACGGCATTGATATATCCGACATAGGTCTGTTCGATTTAAGAAGCCATTTG
GCAATTATTCCTCAGGATGCACAAGCTTTTGAAGGTACAGTAAAGACCAATTTGGACCCTTTCAATCGTTATTC
AGAAGATGAACTTAAAAGGGCTGTTGAGCAGGCACATTTAAAGCCTCATCTGGAAAAAATGCTGCACAGTAA
ACCAAGAGGTGATGATTCTAATGAAGAGGATGGCAATGTTAATGATATTCTGGATGTCAAGATTAATGAGAA
CGGTAGTAACTTGTCAGTGGGGCAAAGACAACTACTATGTTTGGCAAGAGCGCTGCTAAACCGTTCCAAAATA
TTGGTCCTTGATGAAGCAACGGCTTCTGTGGATATGGAAACCGATAAAATTATCCAAGACACTATAAGAAGAG
AATTTAAGGACCGTACCATCTTAACAATTGCACATCGTATCGACACTGTATTGGACAGTGATAAGATAATTGTT
CTTGACCAGGGTAGTGTGAGGGAATTCGATTCACCCTCGAAATTGTTATCCGATAAAACGTCTATTTTTTACAG
TCTTTGTGAGAAAGGTGGGTATTTGAAATAA.
As used herein, the term “T4_Fungal_2” refers to an ABC-transporter having the
following amino acid sequence (SEQ ID NO: 4):
MSSLEVVDGCPYGYRPYPDSGTNALNPCFISVISAWQAVFFLLIGSYQLWKLYKNNKVPPRFKNFPTLPSKINSRHLT
HLTNVCFQSTLIICELALVSQSSDRVYPFILKKALYLNLLFNLGISLPTQYLAYFKSTFSMGNQLFYYMFQILLQLFLILQR
YYHGSSNERLTVISGQTAMILEVLLLFNSVAIFIYDLCIFEPINELSEYYKKNGWYPPVHVLSYITFIWMNKLIVETYRNK
KIKDPNQLPLPPVDLNIKSISKEFKANWELEKWLNRNSLWRAIWKSFGRTISVAMLYETTSDLLSVVQPQFLRIFIDG
FNPETSSKYPPLNGVFIALTLFVISVVSVFLTNQFYIGIFEAGLGIRGSLASLVYQKSLRLTLAERNEKSTGDILNLMSVD
VLRIQRFFENAQTIIGAPIQIIVVLTSLYWLLGKAVVGGLVTMAIMMPINAFLSRKVKKLSKTQMKYKDMRIKTITELL
NAIKSIKLYAWEEPMMARLNHVRNDMELKNFRKIGIVSNLIYFAWNCVPLMVTCSTFGLFSLFSDSPLSPAIVFPSLS
LFNILNSAIYSVPSMINTIIETSVSMERLKSFLLSDEIDDSFIERIDPSADERALPAIEMNNITFLWKSKEVLASSQSGDN
LRTDEESIIGSSQIALKNIDHFEAKRGDLVCVVGRVGAGKSTFLKAILGQLPCMSGSRDSIPPKLIIRSSSVAYCSQESW
IMNASVRENILFGHKFDQNYYDLTIKACQLLPDLKILPDGDETLVGEKGISLSGGQKARLSLARAVYSRADIYLLDDILS
AVDAEVSKNIIEYVLIGKTALLKNKTIILTTNTVSILKHSQMIYALENGEIVEQGNYEDVMNRKNNTSKLKKLLEEFDSP
IDNGNESDVQTEHRSESEVDEPLQLKVTESETEDEVVTESELELIKANSRRASLATLRPRPFVGAQLDSVKKTAQEAE
KTEVGRVKTKVYLAYIKACGVLGVVLFFLFMILTRVFDLAENFWLKYWSESNEKNGSNERVWMFVGVYSLIGVASA
AFNNLRSIMMLLYCSIRGSKKLHESMAKSVIRSPMTFFETTPVGRIINRFSSDMDAVDSNLQYIFSFFFKSILTYLVTVI
LVGYNMPWFLVFNMFLVVIYIYYQTFYIVLSRELKRLISISYSPIMSLMSESLNGYSIIDAYDHFERFIYLNYEKIQYNVD
FVFNFRSTNRWLSVRLQTIGATIVLATAILALATMNTKRQLSSGMVGLLMSYSLEVTGSLTWIVRTTVMIETNIVSVE
RIVEYCELPPEAQSINPEKRPDENWPSKGGIEFKNYSTKYRENLDPVLNNINVKIEPCEKVGIVGRTGAGKSTLSLALF
RILEPTEGKIIIDGIGISDIGLFDLRSHLAIIPQDAQAFEGTVKTNLDPFNRYSEDELKRAVEQAHLKPHLEKMLHSKPR
GDDSNEEDGNVNDILDVKINENGSNLSVGQRQLLCLARALLNRSKILVLDEATASVDMETDKIIQDTIRREFKDRTIL
TIAHRIDTVLDSDKIIVLDQGSVREFDSPSKLLSDKTSIFYSLCEKGGYLK*;
and
encoded by the following nucleic acid sequence (SEQ ID NO: 23):
ATGTCTTCACTAGAAGTGGTAGATGGGTGCCCCTATGGATACCGACCATATCCAGATAGTGGCACAAATGCAT
TAAATCCATGTTTTATATCAGTAATATCCGCCTGGCAAGCCGTCTTTTTCCTATTGATTGGTAGCTATCAATTGT
GGAAACTTTATAAGAACAATAAAGTACCACCCAGATTTAAGAACTTTCCTACATTACCAAGTAAAATCAACAGT
CGACATCTAACGCATTTGACCAATGTTTGCTTTCAGTCCACGCTTATAATTTGTGAACTGGCCTTGGTATCCCAA
TCTAGCGATAGGGTTTATCCATTTATACTAAAGAAGGCTCTGTACTTGAATCTCCTTTTCAATTTGGGTATTTCT
CTCCCTACTCAATACTTAGCTTATTTTAAAAGTACATTTTCAATGGGCAACCAGCTTTTCTATTACATGTTTCAAA
TTCTTCTACAGCTCTTCTTGATATTGCAGAGGTACTATCATGGTTCTAGTAACGAAAGGCTTACTGTTATTAGCG
GACAAACTGCTATGATTTTAGAAGTGCTCCTTCTTTTCAATTCTGTGGCAATTTTTATTTATGATCTATGCATTTT
TGAGCCAATTAACGAATTATCTGAATACTACAAGAAAAATGGGTGGTATCCCCCCGTTCATGTACTATCCTATA
TTACATTTATCTGGATGAACAAACTGATTGTGGAAACTTACCGTAACAAGAAAATCAAAGATCCTAACCAGTTA
CCATTGCCGCCAGTAGATCTGAATATTAAGTCGATAAGTAAGGAATTTAAGGCTAACTGGGAATTGGAAAAAT
GGTTGAATAGAAATTCTCTTTGGAGGGCCATTTGGAAGTCATTTGGTAGGACTATTTCTGTGGCTATGCTGTAT
GAAACGACATCTGATTTACTTTCTGTAGTACAGCCCCAGTTTCTACGGATATTCATAGATGGTTTGAACCCGGA
AACATCTTCTAAATATCCTCCTTTAAATGGTGTATTTATTGCTCTAACCCTTTTCGTAATCAGCGTGGTTTCTGTG
TTCCTCACCAATCAATTTTATATTGGAATTTTTGAGGCTGGTTTGGGGATAAGAGGCTCTTTAGCTTCTTTAGTG
TATCAGAAGTCCTTAAGATTGACGCTAGCAGAGCGTAACGAAAAATCTACTGGTGACATCTTAAATTTGATGT
CTGTGGATGTGTTAAGGATCCAGCGGTTTTTCGAAAATGCCCAAACCATTATTGGCGCTCCTATTCAGATTATT
GTTGTATTAACTTCCCTGTACTGGTTGCTAGGAAAGGCTGTTATTGGAGGGTTGGTTACTATGGCTATTATGAT
GCCTATCAATGCCTTCTTATCTAGAAAGGTAAAAAAGCTATCAAAAACTCAAATGAAGTATAAGGACATGAGA
ATCAAGACTATTACAGAGCTTTTGAATGCTATAAAATCTATTAAATTATACGCCTGGGAGGAACCTATGATGGC
AAGATTGAATCATGTTCGTAATGATATGGAGTTGAAAAATTTTCGGAAAATTGGTATAGTGAGCAATCTGATA
TATTTTGCGTGGAATTGTGTACCTTTAATGGTGACATGTTCCACATTTGGCTTATTTTCTTTATTTAGTGATTCTC
CGTTATCTCCTGCCATTGTCTTCCCTTCATTATCTTTATTTAATATTTTGAACAGTGCCATCTATTCCGTTCCATCC
ATGATAAATACCATTATAGAGACAAGCGTTTCTATGGAAAGATTAAAGTCATTCCTACTTAGTGACGAAATTG
ATGATTCGTTCATCGAACGTATTGATCCTTCAGCGGATGAAAGAGCGTTACCTGCTATAGAGATGAATAATATT
ACATTTTTATGGAAATCAAAAGAAGTATTAACATCTAGCCAATCTGGAGATAATTTGAGGACAGATGAAGAGT
CTATTATCGGATCTTCTCAAATTGCGTTGAAGAATATCGATCATTTTGAAGCAAAAAGGGGTGATTTAGTTTGT
GTTGTTGGTCGGGTAGGAGCTGGTAAATCAACATTTTTGAAGGCAATTCTTGGTCAACTTCCTTGCATGAGTG
GTTCTAGGGACTCGATACCACCTAAACTGATCATTAGATCATCGTCTGTAGCCTACTGTTCACAAGAATCCTGG
ATAATGAACGCATCTGTAAGAGAAAACATTCTATTTGGTCACAAGTTCGACCAAGATTATTATGACCTCACTAT
TAAAGCATGTCAATTGCTACCCGATTTGAAAATACTACCAGATGGTGATGAAACTTTGGTAGGTGAAAAGGGC
ATTTCCCTATCAGGCGGTCAGAAGGCCCGTCTTTCATTAGCCAGAGCGGTGTACTCGAGAGCAGATATTTATTT
GTTGGATGACATTTTATCTGCTGTTGATGCAGAAGTTAGTAAAAATATTATTGAATATGTTTTGATCGGAAAGA
CGGCTTTATTAAAAAATAAAACAATTATTTTAACTACCAATACTGTATCAATTTTAAAACATTCGCAGATGATAT
ATGCGCTAGAAAACGGTGAAATTGTTGAACAAGGGAATTATGAGGATGTAATGAACCGTAAGAACAATACTT
CAAAACTGAAAAAATTACTAGAGGAATTTGATTCTCCGATTGATAATGGAAATGAAAGCGATGTCCAAACTGA
ACACCGATCCGAAAGTGAAGTGGATGAACCTCTGCAGCTTAAAGTAACTGAATCAGAAACTGAGGATGAGGT
TGTTACTGAGAGTGAATTAGAACTAATCAAAGCCAATTCTAGAAGAGCTTCTCTAGCTACGCTAAGACCTAGA
CCCTTTGTGGGAGCACAATTGGATTCCGTGAAGAAAACGGCGCAAAAGGCCGAGAAGACAGAGGTGGGAAG
AGTCAAAACAAAGATTTATCTTGCGTATATTAAGGCTTGTGGAGTTTTAGGTGTTGTTTTATTTTTCTTGTTTAT
GATATTAACAAGGGTTTTCGACTTAGCAGAGAATTTTTGGTTAAAGTACTGGTCAGAATCTAATGAAAAAAAT
GGTTCAAATGAAAGGGTTTGGATGTTTGTTGGTGTGTATTCCTTAATCGGAGTAGCATCGGCCGCATTCAATA
ATTTACGGAGTATTATGATGCTACTGTATTGTTCTATTAGGGGTTCTAAGAAACTGCATGAAAGCATGGCCAA
ATCTGTAATTAGAAGTCCTATGACTTTCTTTGAGACTACACCAGTTGGAAGGATCATAAACAGGTTCTCATCTG
ATATGGATGCAGTGGACAGTAATCTACAGTACATTTTCTCCTTTTTTTTCAAATCAATACTAACCTATTTGGTTA
CTGTTATATTAGTCGGGTACAATATGCCATGGTTTTTAGTGTTCAATATGTTTTTGGTGGTTATCTATATTTACT
ATCAAACATTTTACATTGTGCTATCTAGGGAGCTAAAAAGATTGATCAGTATATCTTACTCTCCGATTATGTCCT
TAATGAGTGAGAGCTTGAACGGTTATTCTATTATTGATGCATACGATCATTTTGAGAGATTCATCTATCTAAAT
TATGAAAAAATCCAATACAACGTTGATTTTGTCTTCAACTTTAGATCAACGAATAGATGGTTATCCGTGAGATT
GCAAACTATTGGTGCTACAATTGTTTTGGCTACTGCAATCTTAGCACTAGCAACAATGAATACTAAAAGGCAAC
TAAGTTCGGGTATGGTTGGTCTACTAATGAGCTATTCATTAGAGGTTACAGGTTCATTGACTTGGATTGTAAG
GACAACTGTGACGATTGAAACCAACATTGTATCAGTGGAGAGAATTGTTGAGTACTGCGAATTACCACCTGAA
GCACAGTCCATTAACCCTGAAAAGAGGCCAGATGAAAATTGGCCATCAAAGGGTGGTATTGAATTCAAAAAC
TATTCCACAAAATACAGAGAAAATTTGGATCCAGTGCTGAATAATATTAACGTGAAGATTGAGCCATGTGAAA
AGGTTGGGATTGTTGGCAGAACAGGTGCAGGGAAGTCTACACTGAGCCTGGCATTATTTAGAATACTAGAAC
CTACCGAAGGTAAAATTATTATTGACGGCATTGATATATCCGACATAGGTCTGTTCGATTTAAGAAGCCATTTG
GCAATTATTCCTCAGGATGCACAAGCTTTTGAAGGTACAGTAAAGACCAATTTGGACCCTTTCAATCGTTATTC
AGAAGATGAACTTAAAAGGGCTGTTGAGCAGGCACATTTAAAGCCTCATCTGGAAAAAATGCTGCACAGTAA
ACCAAGAGGTGATGATTCTAATGAAGAGGATGGCAATGTTAATGATATTCTGGATGTCAAGATTAATGAGAA
CGGTAGTAACTTGTCAGTGGGGCAAAGACAACTACTATGTTTGGCAAGAGCGCTGCTAAACCGTTCCAAAATA
TTGGTCCTTGATGAAGCAACGGCTTCTGTGGATATGGAAACCGATAAAATTATCCAAGACACTATAAGAAGAG
AATTTAAGGACCGTACCATCTTAACAATTGCACATCGTATCGACACTGTATTGGACAGTGATAAGATAATTGTT
CTTGACCAGGGTAGTGTGAGGGAATTCGATTCACCCTCGAAATTGTTATCCGATAAAACGTCTATTTTTTACAG
TCTTTGTGAGAAAGGTGGGTATTTGAAATAA.
As used herein, the term “T4_Fungal_3” refers to an ABC-transporter having the
following amino acid sequence (SEQ ID NO: 5):
MNSYNESAPTGCSFWDNDDISPCIRKSLLDSYLPAAIVVGSLLYLLLIGAQQIKTHRKLYAKDETQPLLEPANGSPTDY
SNTYGTIDYEEEQSTAELTTSQKHFDISRLEPLKDDGTPLGLVKYVQRDGWEKVKLILEFVILIFQLVIAVVALFVPSLN
QEWEGYKLTPIVRVFVWIFLFALGSIRALNKSGPFPLANISLLYYIVNIVPSALSFRSVLIHPQNSQLVNYYYSFQFINNT
LLFLLLGSARVFDHPSVLFDTDDGVKPSPENNSNFFEIVTYSWIDPLIFKAYKTPLQFNDIWGLRIDDYAYFLLRRFKD
LGFTRTFTYKIFYFSKGDLAAQALWASIDSMLIFGPSLLLKRILEYVDNPGMTSRNMAWLYVLTMFFIQISDSLVSGR
SLYLGRRVCIRMKALIIGEVYAKALRRRMTSPEELIEEVDPKDGKAPIADQTSKEESKSTELGGIINLMAVDASKVSEL
CSYLHFFVNSFFMIIVAVTLLYRLLGWSALAGSSSILILLPLNYKLASKIGEFQKEMLGITDNRIQKLNEAFQSIRIIKFFA
WEENFAKEIMKVRNEEIRYLRYRVIVWTCSAFVWFITPTLVTLISFYFYVVFQGKILTTPVAFTALSLFNLLRSPLDQLS
DMLSFMVQSKVSLDRVQKFLEEQESDKYEQLTHTRGANSPEVGFENATLSWNKGSKNDFQLKDIDIAFKVGKLNVI
IGPTGSGKTSLLLGLLGEMQLTNGKIFLPGSTPRDELIPNPETGMTEAVAYCSQIAWLLNDTVKNNIVFAAPFNQQR
YDAVIDACGLTRDLKVLDAGDATEIGEKGITLSGGQKQRVSLARALYSNARHVLLDDCLSAVDSHTAAWIYENCITG
PLMKDRTCILVSHNVALTVRDAAWIVAMDNGRVLEQGTCEDLLSSGSLGHDDLVSTVISSRSQSSVNLKQLNVSDT
SEIHQKLKKIAESDKADQLDEERLSPRGKLIEDETKSSGAVSWEVYKFYGRAFGGVFIWFVFVAAFAASQGSNIMQS
VWLKIWAAANDKLVSPAFTMSIDRSLNALKEGFRASVASVEWSRPLGGEMFRVYGEESSHSSGYYITIYALIGLSYAL
ISAFRVYVVFMGGIVASNKIFEDMLTKIFNAKLRFFDSTPIGRIMNRFSKDTESIDQELAPYAEGFIVSVLQCGATILLI
CIITPGFIVFAAFIVIIYYYIGALYLASSRELKRYDSITVSPIHQHFSETLVGVTTIRAYGDERRFMRQNLEKIDNNNRSFF
YLWVANRWLALRVDFVGALVSLLSAAFVMLSIGHIDAGMAGLSLSYAIAFTQSALWVVRLYSVVEMNMNSVERLE
EYLNIDQEPDREIPDNKPPSSWPETGEIEVDDVSLRYAPSLPKVIKNVSFKVEPRSKIGIVGRTGAGKSTIITAFFRFVD
PESGSIKIDGIDITSIGLKDLRNAVTIIPQDPTLFTGTIRSNLDPFNQYSDAEIFESLKRVNLVSTDEPTSGSSSDNIEDSN
ENVNKFLNLNNTVSEGGSNLSQGQRQLTCLARSLLKSPKIILLDEATASIDYNTDSKIQTTIREEFSDSTILTIAHRLRSII
DYDKILVMDAGRVVEYDDPYKLISDQNSLFYSMCSNSGELDTLVKLAKEAFIAKRNKK*;
and
encoded by the following nucleic acid sequence (SEQ ID NO: 24):
ATGTCTTCACTAGAAGTGGTAGATGGGTGCCCCTATGGATACCGACCATATCCAGATAGTGGCACAAATGCAT
TAAATCCATGTTTTATATCAGTAATATCCGCCTGGCAAGCCGTCTTTTTCCTATTGATTGGTAGCTATCAATTGT
GGAAACTTTATAAGAACAATAAAGTACCACCCAGATTTAAGAACTTTCCTACATTACCAAGTAAAATCAACAGT
CGACATCTAACGCATTTGACCAATGTTTGCTTTCAGTCCACGCTTATAATTTGTGAACTGGCCTTGGTATCCCAA
TCTAGCGATAGGGTTTATCCATTTATACTAAAGAAGGCTCTGTACTTGAATCTCCTTTTCAATTTGGGTATTTCT
CTCCCTACTCAATACTTAGCTTATTTTAAAAGTACATTTTCAATGGGCAACCAGCTTTTCTATTACATGTTTCAAA
TTCTTCTACAGCTCTTCTTGATATTGCAGAGGTACTATCATGGTTCTAGTAACGAAAGGCTTACTGTTATTAGCG
GACAAACTGCTATGATTTTAGAAGTGCTCCTTCTTTTCAATTCTGTGGCAATTTTTATTTATGATCTATGCATTTT
TGAGCCAATTAACGAATTATCTGAATACTACAAGAAAAATGGGTGGTATCCCCCCGTTCATGTACTATCCTATA
TTACATTTATCTGGATGAACAAACTGATTGTGGAAACTTACCGTAACAAGAAAATCAAAGATCCTAACCAGTTA
CCATTGCCGCCAGTAGATCTGAATATTAAGTCGATAAGTAAGGAATTTAAGGCTAACTGGGAATTGGAAAAAT
GGTTGAATAGAAATTCTCTTTGGAGGGCCATTTGGAAGTCATTTGGTAGGACTATTTCTGTGGCTATGCTGTAT
GAAACGACATCTGATTTACTTTCTGTAGTACAGCCCCAGTTTCTACGGATATTCATAGATGGTTTGAACCCGGA
AACATCTTCTAAATATCCTCCTTTAAATGGTGTATTTATTGCTCTAACCCTTTTCGTAATCAGCGTGGTTTCTGTG
TTCCTCACCAATCAATTTTATATTGGAATTTTTGAGGCTGGTTTGGGGATAAGAGGCTCTTTAGCTTCTTTAGTG
TATCAGAAGTCCTTAAGATTGACGCTAGCAGAGCGTAACGAAAAATCTACTGGTGACATCTTAAATTTGATGT
CTGTGGATGTGTTAAGGATCCAGCGGTTTTTCGAAAATGCCCAAACCATTATTGGCGCTCCTATTCAGATTATT
GTTGTATTAACTTCCCTGTACTGGTTGCTAGGAAAGGCTGTTATTGGAGGGTTGGTTACTATGGCTATTATGAT
GCCTATCAATGCCTTCTTATCTAGAAAGGTAAAAAAGCTATCAAAAACTCAAATGAAGTATAAGGACATGAGA
ATCAAGACTATTACAGAGCTTTTGAATGCTATAAAATCTATTAAATTATACGCCTGGGAGGAACCTATGATGGC
AAGATTGAATCATGTTCGTAATGATATGGAGTTGAAAAATTTTCGGAAAATTGGTATAGTGAGCAATCTGATA
TATTTTGCGTGGAATTGTGTACCTTTAATGGTGACATGTTCCACATTTGGCTTATTTTCTTTATTTAGTGATTCTC
CGTTATCTCCTGCCATTGTCTTCCCTTCATTATCTTTATTTAATATTTTGAACAGTGCCATCTATTCCGTTCCATCC
ATGATAAATACCATTATAGAGACAAGCGTTTCTATGGAAAGATTAAAGTCATTCCTACTTAGTGACGAAATTG
ATGATTCGTTCATCGAACGTATTGATCCTTCAGCGGATGAAAGAGCGTTACCTGCTATAGAGATGAATAATATT
ACATTTTTATGGAAATCAAAAGAAGTATTAACATCTAGCCAATCTGGAGATAATTTGAGGACAGATGAAGAGT
CTATTATCGGATCTTCTCAAATTGCGTTGAAGAATATCGATCATTTTGAAGCAAAAAGGGGTGATTTAGTTTGT
GTTGTTGGTCGGGTAGGAGCTGGTAAATCAACATTTTTGAAGGCAATTCTTGGTCAACTTCCTTGCATGAGTG
GTTCTAGGGACTCGATACCACCTAAACTGATCATTAGATCATCGTCTGTAGCCTACTGTTCACAAGAATCCTGG
ATAATGAACGCATCTGTAAGAGAAAACATTCTATTTGGTCACAAGTTCGACCAAGATTATTATGACCTCACTAT
TAAAGCATGTCAATTGCTACCCGATTTGAAAATACTACCAGATGGTGATGAAACTTTGGTAGGTGAAAAGGGC
ATTTCCCTATCAGGCGGTCAGAAGGCCCGTCTTTCATTAGCCAGAGCGGTGTACTCGAGAGCAGATATTTATTT
GTTGGATGACATTTTATCTGCTGTTGATGCAGAAGTTAGTAAAAATATTATTGAATATGTTTTGATCGGAAAGA
CGGCTTTATTAAAAAATAAAACAATTATTTTAACTACCAATACTGTATCAATTTTAAAACATTCGCAGATGATAT
ATGCGCTAGAAAACGGTGAAATTGTTGAACAAGGGAATTATGAGGATGTAATGAACCGTAAGAACAATACTT
CAAAACTGAAAAAATTACTAGAGGAATTTGATTCTCCGATTGATAATGGAAATGAAAGCGATGTCCAAACTGA
ACACCGATCCGAAAGTGAAGTGGATGAACCTCTGCAGCTTAAAGTAACTGAATCAGAAACTGAGGATGAGGT
TGTTACTGAGAGTGAATTAGAACTAATCAAAGCCAATTCTAGAAGAGCTTCTCTAGCTACGCTAAGACCTAGA
CCCTTTGTGGGAGCACAATTGGATTCCGTGAAGAAAACGGCGCAAAAGGCCGAGAAGACAGAGGTGGGAAG
AGTCAAAACAAAGATTTATCTTGCGTATATTAAGGCTTGTGGAGTTTTAGGTGTTGTTTTATTTTTCTTGTTTAT
GATATTAACAAGGGTTTTCGACTTAGCAGAGAATTTTTGGTTAAAGTACTGGTCAGAATCTAATGAAAAAAAT
GGTTCAAATGAAAGGGTTTGGATGTTTGTTGGTGTGTATTCCTTAATCGGAGTAGCATCGGCCGCATTCAATA
ATTTACGGAGTATTATGATGCTACTGTATTGTTCTATTAGGGGTTCTAAGAAACTGCATGAAAGCATGGCCAA
ATCTGTAATTAGAAGTCCTATGACTTTCTTTGAGACTACACCAGTTGGAAGGATCATAAACAGGTTCTCATCTG
ATATGGATGCAGTGGACAGTAATCTACAGTACATTTTCTCCTTTTTTTTCAAATCAATACTAACCTATTTGGTTA
CTGTTATATTAGTCGGGTACAATATGCCATGGTTTTTAGTGTTCAATATGTTTTTGGTGGTTATCTATATTTACT
ATCAAACATTTTACATTGTGCTATCTAGGGAGCTAAAAAGATTGATCAGTATATCTTACTCTCCGATTATGTCCT
TAATGAGTGAGAGCTTGAACGGTTATTCTATTATTGATGCATACGATCATTTTGAGAGATTCATCTATCTAAAT
TATGAAAAAATCCAATACAACGTTGATTTTGTCTTCAACTTTAGATCAACGAATAGATGGTTATCCGTGAGATT
GCAAACTATTGGTGCTACAATTGTTTTGGCTACTGCAATCTTAGCACTAGCAACAATGAATACTAAAAGGCAAC
TAAGTTCGGGTATGGTTGGTCTACTAATGAGCTATTCATTAGAGGTTACAGGTTCATTGACTTGGATTGTAAG
GACAACTGTGACGATTGAAACCAACATTGTATCAGTGGAGAGAATTGTTGAGTACTGCGAATTACCACCTGAA
GCACAGTCCATTAACCCTGAAAAGAGGCCAGATGAAAATTGGCCATCAAAGGGTGGTATTGAATTCAAAAAC
TATTCCACAAAATACAGAGAAAATTTGGATCCAGTGCTGAATAATATTAACGTGAAGATTGAGCCATGTGAAA
AGGTTGGGATTGTTGGCAGAACAGGTGCAGGGAAGTCTACACTGAGCCTGGCATTATTTAGAATACTAGAAC
CTACCGAAGGTAAAATTATTATTGACGGCATTGATATATCCGACATAGGTCTGTTCGATTTAAGAAGCCATTTG
GCAATTATTCCTCAGGATGCACAAGCTTTTGAAGGTACAGTAAAGACCAATTTGGACCCTTTCAATCGTTATTC
AGAAGATGAACTTAAAAGGGCTGTTGAGCAGGCACATTTAAAGCCTCATCTGGAAAAAATGCTGCACAGTAA
ACCAAGAGGTGATGATTCTAATGAAGAGGATGGCAATGTTAATGATATTCTGGATGTCAAGATTAATGAGAA
CGGTAGTAACTTGTCAGTGGGGCAAAGACAACTACTATGTTTGGCAAGAGCGCTGCTAAACCGTTCCAAAATA
TTGGTCCTTGATGAAGCAACGGCTTCTGTGGATATGGAAACCGATAAAATTATCCAAGACACTATAAGAAGAG
AATTTAAGGACCGTACCATCTTAACAATTGCACATCGTATCGACACTGTATTGGACAGTGATAAGATAATTGTT
CTTGACCAGGGTAGTGTGAGGGAATTCGATTCACCCTCGAAATTGTTATCCGATAAAACGTCTATTTTTTACAG
TCTTTGTGAGAAAGGTGGGTATTTGAAATAA.
As used herein, the term “T4_Fungal_4” refers to an ABC-transporter having the
following amino acid sequence (SEQ ID NO: 6):
MSSLEVVDGCPYGYRPYPDSGTNALNPCFISVISAWQAVFFLLIGSYQLWKLYKNNKVPPRFKNFPTLPSKINSRHLT
HLTNVCFQSTLIICELALVSQSSDRVYPFILKKALYLNLLFNLGISLPTQYLAYFKSTFSMGNQLFYYMFQILLQLFLILQR
YYHGSSNERLTVISGQTAMILEVLLLFNSVAIFIYDLCIFEPINELSEYYKKNGWYPPVHVLSYITFIWMNKLIVETYRNK
KIKDPNQLPLPPVDLNIKSISKEFKANWELEKWLNRNSLWRAIWKSFGRTISVAMLYETTSDLLSVVQPQFLRIFIDG
FNPETSSKYPPLNGVFIALTLFVISVVSVFLTNQFYIGIFEAGLGIRGSLASLVYQKSLRLTLAERNEKSTGDILNLMSVD
VLRIQRFFENAQTIIGAPIQIIVVLTSLYWLLGKAVIGGLVTMAIMMPINAFLSRKVKKLSKTQMKYKDMRIKTITELL
NAIKSIKLYAWEEPMMARLNHVRNDMELKNFRKIGIVSNLIYFAWNCVPLMVTCSTFGLFSLFSDSPLSPAIVFPSLS
LFNILNSAIYSVPSMINTIIETSVSMERLKSFLLSDEIDDSFIERIDPSADERALPAIEMNNITFLWKSKEVLASSQSRDN
LRTDEESIIGSSQIALKNIDHFEAKRGDLVCVVGRVGAGKSTFLKAILGQLPCMSGSRDSIPPKLIIRSSSVAYCSQESW
IMNASVRENILFGHKFDQNYYDLTIKACQLLPDLKILPDGDETLVGEKGISLSGGQKARLSLARAVYSRADIYLLDDILS
AVDAEVSKNIIEYVLIGKTALLKNKTIILTTNTVSILKHSQMIYALENGEIVEQGNYEDVMNRKNNTSKLKKLLEEFDSP
IDNGNESDVQTEHRSESEVDEPLQLKVTESETEDEVVTESELELIKANSRRASLATLRPRPFVGAQLDSVKKTAQEAE
KTEVGRVKTKVYLAYIKACGVLGVVLFFLFMILTRVFDLAENFWLKYWSESNEKNGSNERVWMFVGVYSLIGVASA
AFNNLRSIMMLLYCSIRGSKKLHESMAKSVIRSPMTFFETTPVGRIINRFSSDMDAVDSNLQYIFSFFFKSILTYLVTVI
LVGYNMPWFLVFNMFLVVIYIYYQTFYIVLSRELKRLISISYSPIMSLMSESLNGYSIIDAYDHFERFIYLNYEKIQYNVD
FVFNFRSTNRWLSVRLQTIGATIVLATAILALATMNTKRQLSSGMVGLLMSYSLEVTGSLTWIVRTTVMIETNIVSVE
RIVEYCELPPEAQSINPEKRPDENWPSKGGIEFKNYSTKYRENLDPVLNNINVKIEPCEKVGIVGRTGAGKSTLSLALF
RILEPTEGKIIIDGIDISDIGLFDLRSHLAIIPQDAQAFEGTVKTNLDPFNRYSEDELKRAVEQAHLKPHLEKMLHSKPR
GDDSNEEDGNVNDILDVKINENGSNLSVGQRQLLCLARALLNRSKILVLDEATASVDMETDKIIQDTIRREFKDRTIL
TIAHRIDTVLDSDKIIVLDQGSVREFDSPSKLLSDKTSIFYSLCEKGGYLK*;
and
encoded by the following nucleic acid sequence (SEQ ID NO: 25):
ATGTCTTCACTAGAAGTGGTAGATGGGTGCCCCTATGGATACCGACCATATCCAGATAGTGGCACAAATGCAT
TAAATCCATGTTTTATATCAGTAATATCCGCCTGGCAAGCCGTCTTTTTCCTATTGATTGGTAGCTATCAATTGT
GGAAACTTTATAAGAACAATAAAGTACCACCCAGATTTAAGAACTTTCCTACATTACCAAGTAAAATCAACAGT
CGACATCTAACGCATTTGACCAATGTTTGCTTTCAGTCCACGCTTATAATTTGTGAACTGGCCTTGGTATCCCAA
TCTAGCGATAGGGTTTATCCATTTATACTAAAGAAGGCTCTGTACTTGAATCTCCTTTTCAATTTGGGTATTTCT
CTCCCTACTCAATACTTAGCTTATTTTAAAAGTACATTTTCAATGGGCAACCAGCTTTTCTATTACATGTTTCAAA
TTCTTCTACAGCTCTTCTTGATATTGCAGAGGTACTATCATGGTTCTAGTAACGAAAGGCTTACTGTTATTAGCG
GACAAACTGCTATGATTTTAGAAGTGCTCCTTCTTTTCAATTCTGTGGCAATTTTTATTTATGATCTATGCATTTT
TGAGCCAATTAACGAATTATCTGAATACTACAAGAAAAATGGGTGGTATCCCCCCGTTCATGTACTATCCTATA
TTACATTTATCTGGATGAACAAACTGATTGTGGAAACTTACCGTAACAAGAAAATCAAAGATCCTAACCAGTTA
CCATTGCCGCCAGTAGATCTGAATATTAAGTCGATAAGTAAGGAATTTAAGGCTAACTGGGAATTGGAAAAAT
GGTTGAATAGAAATTCTCTTTGGAGGGCCATTTGGAAGTCATTTGGTAGGACTATTTCTGTGGCTATGCTGTAT
GAAACGACATCTGATTTACTTTCTGTAGTACAGCCCCAGTTTCTACGGATATTCATAGATGGTTTGAACCCGGA
AACATCTTCTAAATATCCTCCTTTAAATGGTGTATTTATTGCTCTAACCCTTTTCGTAATCAGCGTGGTTTCTGTG
TTCCTCACCAATCAATTTTATATTGGAATTTTTGAGGCTGGTTTGGGGATAAGAGGCTCTTTAGCTTCTTTAGTG
TATCAGAAGTCCTTAAGATTGACGCTAGCAGAGCGTAACGAAAAATCTACTGGTGACATCTTAAATTTGATGT
CTGTGGATGTGTTAAGGATCCAGCGGTTTTTCGAAAATGCCCAAACCATTATTGGCGCTCCTATTCAGATTATT
GTTGTATTAACTTCCCTGTACTGGTTGCTAGGAAAGGCTGTTATTGGAGGGTTGGTTACTATGGCTATTATGAT
GCCTATCAATGCCTTCTTATCTAGAAAGGTAAAAAAGCTATCAAAAACTCAAATGAAGTATAAGGACATGAGA
ATCAAGACTATTACAGAGCTTTTGAATGCTATAAAATCTATTAAATTATACGCCTGGGAGGAACCTATGATGGC
AAGATTGAATCATGTTCGTAATGATATGGAGTTGAAAAATTTTCGGAAAATTGGTATAGTGAGCAATCTGATA
TATTTTGCGTGGAATTGTGTACCTTTAATGGTGACATGTTCCACATTTGGCTTATTTTCTTTATTTAGTGATTCTC
CGTTATCTCCTGCCATTGTCTTCCCTTCATTATCTTTATTTAATATTTTGAACAGTGCCATCTATTCCGTTCCATCC
ATGATAAATACCATTATAGAGACAAGCGTTTCTATGGAAAGATTAAAGTCATTCCTACTTAGTGACGAAATTG
ATGATTCGTTCATCGAACGTATTGATCCTTCAGCGGATGAAAGAGCGTTACCTGCTATAGAGATGAATAATATT
ACATTTTTATGGAAATCAAAAGAAGTATTAACATCTAGCCAATCTGGAGATAATTTGAGGACAGATGAAGAGT
CTATTATCGGATCTTCTCAAATTGCGTTGAAGAATATCGATCATTTTGAAGCAAAAAGGGGTGATTTAGTTTGT
GTTGTTGGTCGGGTAGGAGCTGGTAAATCAACATTTTTGAAGGCAATTCTTGGTCAACTTCCTTGCATGAGTG
GTTCTAGGGACTCGATACCACCTAAACTGATCATTAGATCATCGTCTGTAGCCTACTGTTCACAAGAATCCTGG
ATAATGAACGCATCTGTAAGAGAAAACATTCTATTTGGTCACAAGTTCGACCAAGATTATTATGACCTCACTAT
TAAAGCATGTCAATTGCTACCCGATTTGAAAATACTACCAGATGGTGATGAAACTTTGGTAGGTGAAAAGGGC
ATTTCCCTATCAGGCGGTCAGAAGGCCCGTCTTTCATTAGCCAGAGCGGTGTACTCGAGAGCAGATATTTATTT
GTTGGATGACATTTTATCTGCTGTTGATGCAGAAGTTAGTAAAAATATTATTGAATATGTTTTGATCGGAAAGA
CGGCTTTATTAAAAAATAAAACAATTATTTTAACTACCAATACTGTATCAATTTTAAAACATTCGCAGATGATAT
ATGCGCTAGAAAACGGTGAAATTGTTGAACAAGGGAATTATGAGGATGTAATGAACCGTAAGAACAATACTT
CAAAACTGAAAAAATTACTAGAGGAATTTGATTCTCCGATTGATAATGGAAATGAAAGCGATGTCCAAACTGA
ACACCGATCCGAAAGTGAAGTGGATGAACCTCTGCAGCTTAAAGTAACTGAATCAGAAACTGAGGATGAGGT
TGTTACTGAGAGTGAATTAGAACTAATCAAAGCCAATTCTAGAAGAGCTTCTCTAGCTACGCTAAGACCTAGA
CCCTTTGTGGGAGCACAATTGGATTCCGTGAAGAAAACGGCGCAAAAGGCCGAGAAGACAGAGGTGGGAAG
AGTCAAAACAAAGATTTATCTTGCGTATATTAAGGCTTGTGGAGTTTTAGGTGTTGTTTTATTTTTCTTGTTTAT
GATATTAACAAGGGTTTTCGACTTAGCAGAGAATTTTTGGTTAAAGTACTGGTCAGAATCTAATGAAAAAAAT
GGTTCAAATGAAAGGGTTTGGATGTTTGTTGGTGTGTATTCCTTAATCGGAGTAGCATCGGCCGCATTCAATA
ATTTACGGAGTATTATGATGCTACTGTATTGTTCTATTAGGGGTTCTAAGAAACTGCATGAAAGCATGGCCAA
ATCTGTAATTAGAAGTCCTATGACTTTCTTTGAGACTACACCAGTTGGAAGGATCATAAACAGGTTCTCATCTG
ATATGGATGCAGTGGACAGTAATCTACAGTACATTTTCTCCTTTTTTTTCAAATCAATACTAACCTATTTGGTTA
CTGTTATATTAGTCGGGTACAATATGCCATGGTTTTTAGTGTTCAATATGTTTTTGGTGGTTATCTATATTTACT
ATCAAACATTTTACATTGTGCTATCTAGGGAGCTAAAAAGATTGATCAGTATATCTTACTCTCCGATTATGTCCT
TAATGAGTGAGAGCTTGAACGGTTATTCTATTATTGATGCATACGATCATTTTGAGAGATTCATCTATCTAAAT
TATGAAAAAATCCAATACAACGTTGATTTTGTCTTCAACTTTAGATCAACGAATAGATGGTTATCCGTGAGATT
GCAAACTATTGGTGCTACAATTGTTTTGGCTACTGCAATCTTAGCACTAGCAACAATGAATACTAAAAGGCAAC
TAAGTTCGGGTATGGTTGGTCTACTAATGAGCTATTCATTAGAGGTTACAGGTTCATTGACTTGGATTGTAAG
GACAACTGTGACGATTGAAACCAACATTGTATCAGTGGAGAGAATTGTTGAGTACTGCGAATTACCACCTGAA
GCACAGTCCATTAACCCTGAAAAGAGGCCAGATGAAAATTGGCCATCAAAGGGTGGTATTGAATTCAAAAAC
TATTCCACAAAATACAGAGAAAATTTGGATCCAGTGCTGAATAATATTAACGTGAAGATTGAGCCATGTGAAA
AGGTTGGGATTGTTGGCAGAACAGGTGCAGGGAAGTCTACACTGAGCCTGGCATTATTTAGAATACTAGAAC
CTACCGAAGGTAAAATTATTATTGACGGCATTGATATATCCGACATAGGTCTGTTCGATTTAAGAAGCCATTTG
GCAATTATTCCTCAGGATGCACAAGCTTTTGAAGGTACAGTAAAGACCAATTTGGACCCTTTCAATCGTTATTC
AGAAGATGAACTTAAAAGGGCTGTTGAGCAGGCACATTTAAAGCCTCATCTGGAAAAAATGCTGCACAGTAA
ACCAAGAGGTGATGATTCTAATGAAGAGGATGGCAATGTTAATGATATTCTGGATGTCAAGATTAATGAGAA
CGGTAGTAACTTGTCAGTGGGGCAAAGACAACTACTATGTTTGGCAAGAGCGCTGCTAAACCGTTCCAAAATA
TTGGTCCTTGATGAAGCAACGGCTTCTGTGGATATGGAAACCGATAAAATTATCCAAGACACTATAAGAAGAG
AATTTAAGGACCGTACCATCTTAACAATTGCACATCGTATCGACACTGTATTGGACAGTGATAAGATAATTGTT
CTTGACCAGGGTAGTGTGAGGGAATTCGATTCACCCTCGAAATTGTTATCCGATAAAACGTCTATTTTTTACAG
TCTTTGTGAGAAAGGTGGGTATTTGAAATAA.
As used herein, the term “T4_Fungal_5” refers to an ABC-transporter having the
following amino acid sequence (SEQ ID NO: 7):
MTSPGSEKCTPRSDEDLERSEPQLQRRLLTPFLLSKKVPPIPKEDERKPYPYLKTNPLSQILFWWLNPLLRVGYKRTLD
PNDFYYLEHSQDIETTYSNYEMHLARILEKDRAKARAKDPTLTDEDLKNREYPKNAVIKALFLTFKWKYLWSIFLKLLS
DIVLVLNPLLSKALINFVDEKMYNPDMSVGRGVGYAIGVTFMLGTSGILINHFLYLSLTVGAHCKAVLTTAIMNKSFR
ASAKSKHEYPSGRVTSLMSTDLARIDLAIGFQPFAITVPVPIGVAIALLIVNIGVSALAGIAVFLVCIVVISASSKSLLKM
RKGANQYTDARISYMREILQNMRIIKFYSWEDAYEKSVVTERNSEMSIILKMQSIRNFLLALSLSLPAIISMVAFLVLY
GVSNDKNPGNIFSSISLFSVLAQQTMMLPMALATGADAKIGLERLRQYLQSGDIEKEYEDHEKPGDRDVVLPDNV
AVELNNASFIWEKFDDADDNDGNSEKTKEVVVTSKSSLTDSSHIDKSTDSADGEYIKSVFEGFNNINLTIKKGEFVIIT
GPIGSGKSSLLVALAGFMKKTSGTLGVNGTMLLCGQPWVQNCTVRDNILFGLEYDEARYDRVVEVCALGDDLKM
FTAGDQTEIGERGITLSGGQKARINLARAVYANKDIILLDDVLSAVDARVGKLIVDDCLTSFLGDKTRILATHQLSLIEA
ADRVIYLNGDGTIHIGTVQELLESNEGFLKLMEFSRKSESEDEEDVEAANEKDVSLQKAVSVVQEQDAHAGVLIGQE
ERAVNGIEWDIYKEYLHEGRGKLGIFAIPTIIMLLVLDVFTSIFVNVWLSFWISHKFKARSDGFYIGLYVMFVILSVIWI
TAEFVVMGYFSSTAARRLNLKAMKRVLHTPMHFLDVTPMGRILNRFTKDTDVLDNEIGEQARMFLHPAAYVIGVL
ILCIIYIPWFAIAIPPLAILFTFITNFYIASSREVKRIEAIQRSLVYNNFNEVLNGLQTLKAYNATSRFMEKNKRLLNRMN
EAYLLVIANQRWISVNLDLVSCCFVFLISMLSVFRVFDINASSVGLVVTSVLQIGGLMSLIMRAYTTVENEMNSVERL
CHYANKLEQEAPYIMNETKPRPTWPEHGAIEFKHASMRYREGLPLVLKDLTISVKGGEKIGICGRTGAGKSTIMNAL
YRLTELAEGSITIDGVEISQLGLYDLRSKLAIIPQDPVLFRGTIRKNLDPFGQNDDETLWDALRRSGLVEGSILNTIKSQ
SKDDPNFHKFHLDQTVEDEGANFSLGERQLIALARALVRNSKILILDEATSSVDYETDSKIQKTISTEFSHCTILCIAHRL
KTILTYDRILVLEKGEVEEFDTPRVLYSKNGVFRQMCERSEITSADFV*;
and
encoded by the following nucleic acid sequence (SEQ ID NO: 26):
ATGTCTTCACTAGAAGTGGTAGATGGGTGCCCCTATGGATACCGACCATATCCAGATAGTGGCACAAATGCAT
TAAATCCATGTTTTATATCAGTAATATCCGCCTGGCAAGCCGTCTTTTTCCTATTGATTGGTAGCTATCAATTGT
GGAAACTTTATAAGAACAATAAAGTACCACCCAGATTTAAGAACTTTCCTACATTACCAAGTAAAATCAACAGT
CGACATCTAACGCATTTGACCAATGTTTGCTTTCAGTCCACGCTTATAATTTGTGAACTGGCCTTGGTATCCCAA
TCTAGCGATAGGGTTTATCCATTTATACTAAAGAAGGCTCTGTACTTGAATCTCCTTTTCAATTTGGGTATTTCT
CTCCCTACTCAATACTTAGCTTATTTTAAAAGTACATTTTCAATGGGCAACCAGCTTTTCTATTACATGTTTCAAA
TTCTTCTACAGCTCTTCTTGATATTGCAGAGGTACTATCATGGTTCTAGTAACGAAAGGCTTACTGTTATTAGCG
GACAAACTGCTATGATTTTAGAAGTGCTCCTTCTTTTCAATTCTGTGGCAATTTTTATTTATGATCTATGCATTTT
TGAGCCAATTAACGAATTATCTGAATACTACAAGAAAAATGGGTGGTATCCCCCCGTTCATGTACTATCCTATA
TTACATTTATCTGGATGAACAAACTGATTGTGGAAACTTACCGTAACAAGAAAATCAAAGATCCTAACCAGTTA
CCATTGCCGCCAGTAGATCTGAATATTAAGTCGATAAGTAAGGAATTTAAGGCTAACTGGGAATTGGAAAAAT
GGTTGAATAGAAATTCTCTTTGGAGGGCCATTTGGAAGTCATTTGGTAGGACTATTTCTGTGGCTATGCTGTAT
GAAACGACATCTGATTTACTTTCTGTAGTACAGCCCCAGTTTCTACGGATATTCATAGATGGTTTGAACCCGGA
AACATCTTCTAAATATCCTCCTTTAAATGGTGTATTTATTGCTCTAACCCTTTTCGTAATCAGCGTGGTTTCTGTG
TTCCTCACCAATCAATTTTATATTGGAATTTTTGAGGCTGGTTTGGGGATAAGAGGCTCTTTAGCTTCTTTAGTG
TATCAGAAGTCCTTAAGATTGACGCTAGCAGAGCGTAACGAAAAATCTACTGGTGACATCTTAAATTTGATGT
CTGTGGATGTGTTAAGGATCCAGCGGTTTTTCGAAAATGCCCAAACCATTATTGGCGCTCCTATTCAGATTATT
GTTGTATTAACTTCCCTGTACTGGTTGCTAGGAAAGGCTGTTATTGGAGGGTTGGTTACTATGGCTATTATGAT
GCCTATCAATGCCTTCTTATCTAGAAAGGTAAAAAAGCTATCAAAAACTCAAATGAAGTATAAGGACATGAGA
ATCAAGACTATTACAGAGCTTTTGAATGCTATAAAATCTATTAAATTATACGCCTGGGAGGAACCTATGATGGC
AAGATTGAATCATGTTCGTAATGATATGGAGTTGAAAAATTTTCGGAAAATTGGTATAGTGAGCAATCTGATA
TATTTTGCGTGGAATTGTGTACCTTTAATGGTGACATGTTCCACATTTGGCTTATTTTCTTTATTTAGTGATTCTC
CGTTATCTCCTGCCATTGTCTTCCCTTCATTATCTTTATTTAATATTTTGAACAGTGCCATCTATTCCGTTCCATCC
ATGATAAATACCATTATAGAGACAAGCGTTTCTATGGAAAGATTAAAGTCATTCCTACTTAGTGACGAAATTG
ATGATTCGTTCATCGAACGTATTGATCCTTCAGCGGATGAAAGAGCGTTACCTGCTATAGAGATGAATAATATT
ACATTTTTATGGAAATCAAAAGAAGTATTAACATCTAGCCAATCTGGAGATAATTTGAGGACAGATGAAGAGT
CTATTATCGGATCTTCTCAAATTGCGTTGAAGAATATCGATCATTTTGAAGCAAAAAGGGGTGATTTAGTTTGT
GTTGTTGGTCGGGTAGGAGCTGGTAAATCAACATTTTTGAAGGCAATTCTTGGTCAACTTCCTTGCATGAGTG
GTTCTAGGGACTCGATACCACCTAAACTGATCATTAGATCATCGTCTGTAGCCTACTGTTCACAAGAATCCTGG
ATAATGAACGCATCTGTAAGAGAAAACATTCTATTTGGTCACAAGTTCGACCAAGATTATTATGACCTCACTAT
TAAAGCATGTCAATTGCTACCCGATTTGAAAATACTACCAGATGGTGATGAAACTTTGGTAGGTGAAAAGGGC
ATTTCCCTATCAGGCGGTCAGAAGGCCCGTCTTTCATTAGCCAGAGCGGTGTACTCGAGAGCAGATATTTATTT
GTTGGATGACATTTTATCTGCTGTTGATGCAGAAGTTAGTAAAAATATTATTGAATATGTTTTGATCGGAAAGA
CGGCTTTATTAAAAAATAAAACAATTATTTTAACTACCAATACTGTATCAATTTTAAAACATTCGCAGATGATAT
ATGCGCTAGAAAACGGTGAAATTGTTGAACAAGGGAATTATGAGGATGTAATGAACCGTAAGAACAATACTT
CAAAACTGAAAAAATTACTAGAGGAATTTGATTCTCCGATTGATAATGGAAATGAAAGCGATGTCCAAACTGA
ACACCGATCCGAAAGTGAAGTGGATGAACCTCTGCAGCTTAAAGTAACTGAATCAGAAACTGAGGATGAGGT
TGTTACTGAGAGTGAATTAGAACTAATCAAAGCCAATTCTAGAAGAGCTTCTCTAGCTACGCTAAGACCTAGA
CCCTTTGTGGGAGCACAATTGGATTCCGTGAAGAAAACGGCGCAAAAGGCCGAGAAGACAGAGGTGGGAAG
AGTCAAAACAAAGATTTATCTTGCGTATATTAAGGCTTGTGGAGTTTTAGGTGTTGTTTTATTTTTCTTGTTTAT
GATATTAACAAGGGTTTTCGACTTAGCAGAGAATTTTTGGTTAAAGTACTGGTCAGAATCTAATGAAAAAAAT
GGTTCAAATGAAAGGGTTTGGATGTTTGTTGGTGTGTATTCCTTAATCGGAGTAGCATCGGCCGCATTCAATA
ATTTACGGAGTATTATGATGCTACTGTATTGTTCTATTAGGGGTTCTAAGAAACTGCATGAAAGCATGGCCAA
ATCTGTAATTAGAAGTCCTATGACTTTCTTTGAGACTACACCAGTTGGAAGGATCATAAACAGGTTCTCATCTG
ATATGGATGCAGTGGACAGTAATCTACAGTACATTTTCTCCTTTTTTTTCAAATCAATACTAACCTATTTGGTTA
CTGTTATATTAGTCGGGTACAATATGCCATGGTTTTTAGTGTTCAATATGTTTTTGGTGGTTATCTATATTTACT
ATCAAACATTTTACATTGTGCTATCTAGGGAGCTAAAAAGATTGATCAGTATATCTTACTCTCCGATTATGTCCT
TAATGAGTGAGAGCTTGAACGGTTATTCTATTATTGATGCATACGATCATTTTGAGAGATTCATCTATCTAAAT
TATGAAAAAATCCAATACAACGTTGATTTTGTCTTCAACTTTAGATCAACGAATAGATGGTTATCCGTGAGATT
GCAAACTATTGGTGCTACAATTGTTTTGGCTACTGCAATCTTAGCACTAGCAACAATGAATACTAAAAGGCAAC
TAAGTTCGGGTATGGTTGGTCTACTAATGAGCTATTCATTAGAGGTTACAGGTTCATTGACTTGGATTGTAAG
GACAACTGTGACGATTGAAACCAACATTGTATCAGTGGAGAGAATTGTTGAGTACTGCGAATTACCACCTGAA
GCACAGTCCATTAACCCTGAAAAGAGGCCAGATGAAAATTGGCCATCAAAGGGTGGTATTGAATTCAAAAAC
TATTCCACAAAATACAGAGAAAATTTGGATCCAGTGCTGAATAATATTAACGTGAAGATTGAGCCATGTGAAA
AGGTTGGGATTGTTGGCAGAACAGGTGCAGGGAAGTCTACACTGAGCCTGGCATTATTTAGAATACTAGAAC
CTACCGAAGGTAAAATTATTATTGACGGCATTGATATATCCGACATAGGTCTGTTCGATTTAAGAAGCCATTTG
GCAATTATTCCTCAGGATGCACAAGCTTTTGAAGGTACAGTAAAGACCAATTTGGACCCTTTCAATCGTTATTC
AGAAGATGAACTTAAAAGGGCTGTTGAGCAGGCACATTTAAAGCCTCATCTGGAAAAAATGCTGCACAGTAA
ACCAAGAGGTGATGATTCTAATGAAGAGGATGGCAATGTTAATGATATTCTGGATGTCAAGATTAATGAGAA
CGGTAGTAACTTGTCAGTGGGGCAAAGACAACTACTATGTTTGGCAAGAGCGCTGCTAAACCGTTCCAAAATA
TTGGTCCTTGATGAAGCAACGGCTTCTGTGGATATGGAAACCGATAAAATTATCCAAGACACTATAAGAAGAG
AATTTAAGGACCGTACCATCTTAACAATTGCACATCGTATCGACACTGTATTGGACAGTGATAAGATAATTGTT
CTTGACCAGGGTAGTGTGAGGGAATTCGATTCACCCTCGAAATTGTTATCCGATAAAACGTCTATTTTTTACAG
TCTTTGTGAGAAAGGTGGGTATTTGAAATAA.
As used herein, the term “T4_Fungal_8” refers to an ABC-transporter having the
following amino acid sequence (SEQ ID NO: 8):
MSGSNSNSNLDAISDSCPFWRYDDITECGRVQYINYYLPITLVGVSLLYLFKNAIQHYYRKPQEIKPSVASELLGSNLT
DLPNENKPLLSESTQALYTNPDSNKTGFSLKEEHFSINKVTLTEIHSNKHDAVKIVRRNWLEKLRVFLEWVLCALQLCI
YISVWSKYTNTQEDFPMHASISGLMLWSLLLLVVSLRLANINQNISWINSGPGNLWALSFACYLSLFCGSVLPLRSIYI
GHITDEIASTFYKLQFYLSLTLFLLLFTSQAGNRFAIIYKSTPDITPSPEPIVSIASYITWAWVDKFLWKAHQNYIEMKD
VWGLMVEDYSILVIKRFNHFVQNKTKSRTFSFNLIHFFMKFIAIQGAWATISSVISFVPTMLLRRILEYVEDQSTAPLN
LAWMYIFLMFLARILTAICAAQALFLGRRVCIRMKAIIISEIYSKALRRKISPNSTKEPTDVVDPQELNDKQHVDGDEE
SATTANLGAIINLMAVDAFKVSEICAYLHSFIEAIIMTIVALFLLYRLIGWSALVGSAMIICFLPLNFKLASLLGTLQKKSL
AITDKRIQKLNEAFQAIRIIKFFSWEENFEKDIQNTRDEELNMLLKRSIVWALSSLVWFITPSIVTSASFAVYIYVQGQT
LTTPVAFTALSLFALLRNPLDMLSDMLSFVIQSKVSLDRVQEFLNEEETKKYEQLTVSRNKLGLQNATFTWDKNNQ
DFKLKNLTIDFKIGKLNVIVGPTGSGKTSLLMGLLGEMELLNGKVFVPSLNPREELVVEADGMTNSIAYCSQAAWLL
NDTVRNNILFNAPYNENRYNAVISACGLKRDFEILSAGDQTEIGEKGITLSGGQKQRVSLARSLYSSSRHLLLDDCLSA
VDSHTALWIYENCITGPLMEGRTCVLVSHNVALTLKNADWVIIMENGRVKEQGEPVELLQKGSLGDDSMVKSSILS
RTASSVNISETNSKISSGPKAPAESDNANEESTTCGDRSKSSGKLIAEETKSNGVVSLDVYKWYAVFFGGWKMISFL
CFIFLFAQMISISQAWWLRAWASNNTLKVFSNLGLQTMRPFALSLQGKEASPVTLSAVFPNGSLTTATEPNHSNAY
YLSIYLGIGVFQALCSSSKAIINFVAGIRASRKIFNLLLKNVLYAKLRFFDSTPIGRIMNRFSKDIESIDQELTPYMEGAFG
SLIQCVSTIIVIAYITPQFLIVAAIVMLLFYFVAYFYMSGARELKRLESMSRSPIHQHFSETLVGITTIRAFSDERRFLVDN
MKKIDDNNRPFFYLWVCNRWLSYRIELIGALIVLAAGSFILLNIKSIDSGLAGISLGFAIQFTDGALWVVRLYSNVEM
NMNSVERLKEYTTIEQEPSNVGALVPPCEWPQNGKIEVKDLSLRYAAGLPKVIKNVTFTVDSKCKVGIVGRTGAGKS
TIITALFRFLDPETGYIKIDDVDITTIGLKRLRQSITIIPQDPTLFTGTLKTNLDPYNEYSEAEIFEALKRVNLVSSEELGNPS
TSDSTSVHSANMNKFLDLENEVSEGGSNLSQGQRQLICLARSLLRCPKVILLDEATASIDYNSDSKIQATIREEFSNSTI
LTIAHRLRSIIDYDKILVMDAGEVKEYDHPYSLLLNRDSIFYHMCEDSGELEVLIQLAKESFVKKLNAN;
and
encoded by the following nucleic acid sequence (SEQ ID NO: 27):
ATGTCAGGTTCAAATTCGAATTCAAATCTAGATGCAATAAGTGATTCATGCCCATTTTGGCGCTATGATGATAT
TACAGAGTGTGGAAGAGTGCAGTATATCAATTACTACCTTCCAATAACATTGGTAGGCGTTTCTCTCTTGTATT
TATTCAAAAACGCGATCCAACATTATTACAGAAAGCCTCAAGAAATTAAGCCTAGTGTTGCTTCCGAATTATTG
GGCTCAAATCTCACAGACCTTCCGAATGAAAACAAGCCTTTACTATCGGAGAGTACACAAGCATTATACACTA
ATCCGGATTCGAATAAGACAGGATTCTCTCTAAAAGAGGAGCATTTCTCTATAAATAAAGTTACACTTACGGA
AATTCATTCCAATAAGCATGACGCTGTGAAGATCGTAAGGAGAAACTGGCTTGAAAAATTAAGAGTGTTCTTA
GAATGGGTTCTATGCGCCTTACAACTTTGCATCTACATTTCAGTCTGGTCGAAATACACTAATACCCAAGAGGA
TTTCCCAATGCACGCATCTATCTCAGGTCTAATGTTATGGTCTCTACTCTTGTTAGTAGTGTCATTGAGGTTGGC
AAACATCAACCAGAATATAAGCTGGATCAATTCAGGACCGGGAAACTTATGGGCCCTTTCATTTGCATGTTATC
TATCACTATTCTGCGGATCCGTTTTGCCATTGAGATCTATCTATATCGGTCATATCACAGATGAAATTGCATCAA
CATTTTATAAGTTGCAATTTTACCTAAGTTTGACACTATTCTTGTTACTTTTCACCTCTCAAGCGGGAAATCGGT
TTGCCATTATCTATAAAAGTACACCAGATATAACACCGTCTCCTGAACCTATTGTGTCGATTGCAAGTTATATCA
CTTGGGCATGGGTAGATAAATTTCTTTGGAAAGCGCATCAAAATTATATCGAAATGAAAGATGTTTGGGGTCT
AATGGTGGAAGACTATTCCATTCTCGTAATAAAGAGATTCAATCATTTTGTTCAGAATAAAACCAAGTCTAGGA
CATTTTCATTTAACTTAATCCACTTTTTCATGAAATTTATCGCCATTCAAGGTGCCTGGGCAACAATTTCGTCAG
TTATTAGTTTTGTTCCAACAATGTTGCTCAGACGTATTTTGGAGTATGTTGAAGATCAATCAACTGCTCCATTAA
ATTTGGCTTGGATGTATATTTTTCTTATGTTCCTTGCCAGAATTTTAACTGCCATATGTGCTGCTCAGGCGCTAT
TTTTAGGGAGAAGGGTTTGTATCAGAATGAAGGCTATCATAATTTCTGAAATCTACTCCAAGGCTTTGAGAAG
AAAAATTTCTCCAAATTCCACTAAGGAGCCAACTGATGTCGTTGATCCACAGGAATTAAATGACAAACAACAC
GTTGATGGAGATGAAGAATCAGCAACCACTGCAAATCTTGGTGCTATCATTAATTTGATGGCGGTGGATGCTT
TCAAAGTATCCGAAATATGTGCGTATTTGCACTCCTTTATAGAGGCGATCATCATGACCATTGTTGCATTATTCC
TTTTATATCGGTTAATAGGCTGGTCTGCTTTAGTTGGTAGTGCAATGATTATTTGCTTCTTACCATTGAACTTCA
AACTTGCCAGCTTGTTAGGGACACTCCAAAAGAAATCCTTGGCAATCACAGATAAAAGAATTCAGAAACTAAA
CGAAGCTTTCCAGGCCATTCGTATTATCAAATTCTTCTCTTGGGAAGAGAATTTTGAAAAGGACATACAAAACA
CAAGGGATGAAGAATTAAATATGCTTTTAAAAAGGTCTATCGTTTGGGCTCTTTCTTCTCTTGTTTGGTTCATTA
CCCCCTCTATTGTCACATCCGCTTCTTTTGCAGTCTATATTTATGTGCAAGGCCAAACTTTAACTACTCCGGTAG
CATTTACTGCACTATCTCTATTTGCTCTACTAAGAAATCCGTTAGACATGCTTTCTGATATGTTGTCTTTTGTTAT
TCAATCCAAGGTCTCTTTGGATAGAGTCCAAGAATTTTTAAATGAAGAGGAGACGAAAAAGTATGAGCAATTA
ACCGTATCAAGAAATAAACTTGGGTTGCAAAACGCTACTTTTACATGGGATAAAAATAATCAAGATTTCAAGTT
AAAAAACCTAACTATTGATTTCAAAATTGGGAAATTAAACGTTATTGTAGGTCCAACTGGATCTGGTAAAACAT
CATTGTTAATGGGATTATTGGGTGAAATGGAGCTATTGAACGGAAAAGTTTTCGTCCCTTCGCTCAATCCTAG
GGAAGAGTTGGTTGTAGAGGCCGATGGAATGACTAATTCAATCGCGTACTGCTCCCAAGCTGCCTGGTTGCTA
AATGATACTGTCAGGAACAATATTCTATTCAATGCGCCTTATAATGAGAATAGATATAATGCCGTCATCTCTGC
GTGTGGTTTGAAACGCGACTTCGAGATCTTAAGCGCTGGTGATCAGACAGAGATTGGCGAAAAGGGTATAAC
ACTTTCTGGTGGTCAAAAACAAAGAGTCTCGTTGGCCAGATCATTGTATTCTTCATCAAGACATTTGCTGTTAG
ATGATTGTTTGAGTGCCGTAGACTCGCACACGGCCTTATGGATCTACGAAAATTGTATAACAGGCCCATTAAT
GGAAGGAAGAACATGTGTATTGGTTTCTCACAATGTTGCATTAACTTTAAAAAATGCAGATTGGGTTATCATTA
TGGAAAATGGTAGAGTAAAAGAACAAGGCGAACCAGTAGAATTGCTACAGAAGGGGTCCCTTGGGGATGAC
TCCATGGTGAAATCATCAATTTTGTCCCGTACGGCGTCCTCAGTTAATATTTCAGAAACTAACAGTAAGATTTCT
AGTGGTCCGAAGGCTCCAGCGGAATCGGATAATGCCAATGAGGAGTCCACCACCTGTGGAGATCGTTCAAAG
TCAAGCGGCAAGCTAATCGCTGAAGAAACAAAATCAAACGGTGTTGTTTCCCTGGACGTCTATAAGTGGTATG
CCGTGTTTTTCGGTGGATGGAAGATGATATCATTTTTGTGTTTCATTTTCTTGTTTGCCCAAATGATCAGTATTT
CACAGGCCTGGTGGTTGCGTGCTTGGGCCTCCAACAACACTCTAAAAGTTTTCTCCAACCTTGGATTGCAAACA
ATGAGGCCATTCGCTTTGTCCTTACAAGGAAAAGAAGCTTCTCCTGTGACTCTTAGTGCTGTTTTCCCAAATGG
CAGTCTAACAACAGCCACGGAACCAAATCACTCGAACGCGTATTATCTATCAATATATTTGGGTATTGGTGTAT
TCCAGGCTTTATGTTCATCTTCGAAAGCAATTATAAACTTTGTGGCCGGTATTAGAGCTTCCAGGAAAATATTC
AATTTATTGTTGAAAAATGTGTTATACGCCAAGCTGAGATTTTTTGATTCTACTCCAATAGGAAGAATAATGAA
CAGATTTTCTAAAGACATCGAATCAATAGATCAAGAATTGACTCCTTATATGGAAGGTGCATTTGGTTCCTTAA
TACAATGTGTTTCCACAATTATCGTCATTGCATACATTACTCCCCAATTTTTGATTGTCGCGGCGATTGTCATGT
TATTGTTTTATTTTGTTGCCTACTTTTACATGTCAGGAGCAAGAGAATTAAAGCGTCTTGAATCGATGTCACGCT
CTCCTATTCATCAGCACTTCTCTGAGACTCTTGTGGGTATCACGACTATTCGAGCATTTTCTGACGAGCGGCGT
TTTCTGGTTGATAATATGAAGAAAATTGATGATAATAATAGGCCTTTCTTTTACTTATGGGTCTGTAATAGATG
GCTATCTTACAGAATCGAGCTGATAGGCGCCCTTATTGTTTTGGCTGCAGGTAGTTTCATCTTATTGAACATAA
AATCGATCGATTCTGGTTTGGCCGGTATTTCATTGGGTTTCGCTATACAATTTACCGATGGTGCCCTTTGGGTT
GTTAGGTTATATTCCAACGTTGAAATGAATATGAATTCCGTCGAAAGGTTAAAAGAGTACACCACCATCGAGC
AAGAACCTTCTAACGTTGGTGCCTTGGTACCTCCTTGCGAATGGCCACAAAATGGTAAAATCGAAGTCAAGGA
TTTATCTTTACGCTATGCAGCTGGTCTACCAAAGGTTATAAAAAATGTCACATTCACCGTCGATTCAAAGTGTA
AAGTAGGTATTGTTGGCAGGACTGGTGCTGGTAAATCTACTATTATCACAGCCCTTTTCAGATTCTTAGACCCT
GAAACTGGTTATATCAAAATCGATGACGTTGATATAACAACCATTGGTTTAAAACGTTTGCGCCAATCTATCAC
TATTATTCCACAGGACCCAACCCTTTTCACCGGTACTTTGAAAACCAATCTCGATCCATACAACGAATATTCGG
AAGCTGAAATTTTCGAAGCTCTAAAACGTGTCAACCTTGTTTCCTCAGAAGAACTTGGTAATCCTTCTACTTCG
GATTCAACCTCGGTACATTCAGCAAATATGAATAAGTTTTTGGATTTGGAAAATGAAGTCAGTGAAGGTGGTT
CCAACCTCTCACAAGGACAACGTCAATTGATATGTTTGGCCCGTTCATTATTGCGGTGTCCAAAGGTAATTCTA
CTTGATGAAGCCACAGCTTCAATCGATTATAACTCAGACTCTAAAATCCAGGCTACTATAAGGGAAGAATTCA
GTAATAGTACCATTCTCACGATTGCTCATCGTTTACGATCAATTATTGATTATGATAAAATACTTGTTATGGATG
CTGGGGAGGTTAAAGAATATGATCATCCTTACTCCTTATTGTTGAATCGTGATAGTATATTCTATCATATGTGT
GAAGATAGTGGAGAATTAGAAGTCTTGATACAATTAGCCAAAGAATCATTTGTCAAAAAGCTCAATGCAAATT
GA.

As used herein, the term “parent cell” refers to a cell that has an identical genetic background as a genetically modified host cell disclosed herein except that it does not comprise one or more particular genetic modifications engineered into the modified host cell, for example, one or more modifications selected from the group consisting of heterologous expression of an enzyme of a steviol pathway, heterologous expression of an enzyme of a steviol glycoside pathway, heterologous expression of a geranylgeranyl diphosphate synthase, heterologous expression of a copalyl diphosphate synthase, heterologous expression of a kaurene synthase, heterologous expression of a kaurene oxidase (e.g., Pisum sativum kaurene oxidase), heterologous expression of a steviol synthase (kaurenoic acid hydroxylase), heterologous expression of a cytochrome P450 reductase, heterologous expression of a EUGT11, heterologous expression of a UGT74G1, heterologous expression of a UGT76G1, heterologous expression of a UGT85C2, heterologous expression of a UGT91D, and heterologous expression of a UGT40087 or its variant.

As used herein, the term “naturally occurring” refers to what is found in nature. For example, an ABC-transporter that is present in an organism that can be isolated from a source in nature and that has not been intentionally modified by a human in the laboratory is naturally occurring ABC-transporter. Conversely, as used herein, the term “non-naturally occurring” refers to what is not found in nature but is created by human intervention.

The term “medium” refers to a culture medium and/or fermentation medium.

The term “fermentation composition” refers to a composition which comprises genetically modified host cells and products or metabolites produced by the genetically modified host cells. An example of a fermentation composition is a whole cell broth, which can be the entire contents of a vessel (e.g., a flask, plate, or fermentor), including cells, aqueous phase, and compounds produced from the genetically modified host cells.

As used herein, the term “production” generally refers to an amount of steviol or steviol glycoside produced by a genetically modified host cell provided herein. In some embodiments, production is expressed as a yield of steviol or steviol glycoside by the host cell. In other embodiments, production is expressed as the productivity of the host cell in producing the steviol or steviol glycoside.

As used herein, the term “productivity” refers to production of a steviol or steviol glycoside by a host cell, expressed as the amount of steviol or steviol glycoside produced (by weight) per amount of fermentation broth in which the host cell is cultured (by volume) over time (per hour).

As used herein, the term “yield” refers to production of a steviol or steviol glycoside by a host cell, expressed as the amount of steviol or steviol glycoside produced per amount of carbon source consumed by the host cell, by weight.

As used herein, the term “an undetectable level” of a compound (e.g., Reb M, steviol glycosides, or other compounds) means a level of a compound that is too low to be measured and/or analyzed by a standard technique for measuring the compound. For instance, the term includes the level of a compound that is not detectable by the analytical methods known in the art.

The term “kaurene” refers to the compound kaurene, including any stereoisomer of kaurene. In particular embodiments, the term refers to the enantiomer known in the art as ent-kaurene. In particular embodiments, the term refers to the compound according to the following structure:

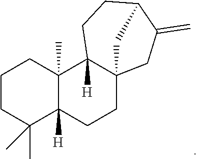

The term “kaurenol” refers to the compound kaurenol, including any stereoisomer of kaurenol. In particular embodiments, the term refers to the enantiomer known in the art as ent-kaurenol. In particular embodiments, the term refers to the compound according to the following structure.

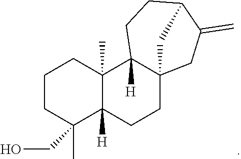

The term “kaurenal” refers to the compound kaurenal, including any stereoisomer of kaurenal. In particular embodiments, the term refers to the enantiomer known in the art as ent-kaurenal. In particular embodiments, the term refers to the compound according to the following structure.

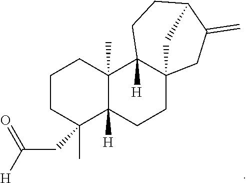

The term “kaurenoic acid” refers to the compound kaurenoic acid, including any stereoisomer of kaurenoic acid. In particular embodiments, the term refers to the enantiomer known in the art as ent-kaurenoic acid. In particular embodiments, the term refers to the compound according to the following structure.

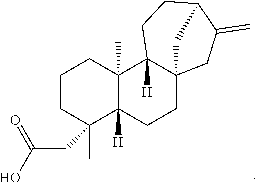

The term “steviol” refers to the compound steviol, including any stereoisomer of steviol. In particular embodiments, the term refers to the compound according to the following structure.

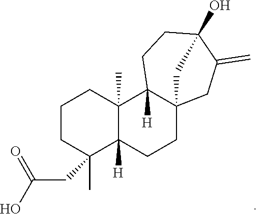

As used herein, the term “steviol glycoside(s)” refers to a glycoside of steviol, including, but not limited to, naturally occurring steviol glycosides, e.g. steviolmonoside, steviolbioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, synthetic steviol glycosides, e.g. enzymatically glucosylated steviol glycosides and combinations thereof.

As used herein, the term “Rebaudioside M” refers to the compound of the following structure.

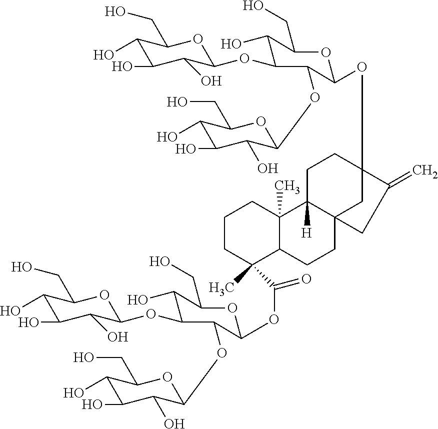

As used herein, the term “variant” refers to a polypeptide differing from a specifically recited “reference” polypeptide (e.g., a wild-type sequence) by amino acid insertions, deletions, mutations, and/or substitutions, but retains an activity that is substantially similar to the reference polypeptide. In some embodiments, the variant is created by recombinant DNA techniques or by mutagenesis. In some embodiments, a variant polypeptide differs from its reference polypeptide by the substitution of one basic residue for another (i.e. Arg for Lys), the substitution of one hydrophobic residue for another (i.e. Leu for Ile), or the substitution of one aromatic residue for another (i.e. Phe for Tyr), etc. In some embodiments, variants include analogs wherein conservative substitutions resulting in a substantial structural analogy of the reference sequence are obtained. Examples of such conservative substitutions, without limitation, include glutamic acid for aspartic acid and vice-versa; glutamine for asparagine and vice-versa; serine for threonine and vice-versa; lysine for arginine and vice-versa; or any of isoleucine, valine or leucine for each other.

As used herein, the term “sequence identity” or “percent identity,” in the context or two or more nucleic acid or protein sequences, refers to two or more sequences or subsequences that are the same or have a specified percentage of amino acid residues or nucleotides that are the same. For example, the sequence can have a percent identity of at least 60%, at least 65%, at least 70%, at least 75%, at least 80%, at least 85%, at least 90%, at least 91% at least 92%, at least 93%, at least 94%, at least 95%, at least 96%, at least 97%, at least 98%, at least 99%, or higher identity over a specified region to a reference sequence when compared and aligned for maximum correspondence over a comparison window, or designated region as measured using a sequence comparison algorithm or by manual alignment and visual inspection. For example, percent of identity is determined by calculating the ratio of the number of identical nucleotides (or amino acid residues) in the sequence divided by the length of the total nucleotides (or amino acid residues) minus the lengths of any gaps.

For convenience, the extent of identity between two sequences can be ascertained using computer programs and mathematical algorithms known in the art. Such algorithms that calculate percent sequence identity generally account for sequence gaps and mismatches over the comparison region. Programs that compare and align sequences, like Clustal W (Thompson et al., (1994) Nucleic Acids Res., 22: 4673-4680), ALIGN (Myers et al., (1988) CABIOS, 4: 11-17), FASTA (Pearson et al., (1988) PNAS, 85:2444-2448; Pearson (1990), Methods Enzymol., 183: 63-98) and gapped BLAST (Altschul et al., (1997) Nucleic Acids Res., 25: 3389-3402) are useful for this purpose. The BLAST or BLAST 2.0 (Altschul et al., J. Mol. Biol. 215:403-10, 1990) is available from several sources, including the National Center for Biological Information (NCBI) and on the Internet, for use in connection with the sequence analysis programs BLASTP, BLASTN, BLASTX, TBLASTN, and TBLASTX. Additional information can be found at the NCBI web site.

In certain embodiments, the sequence alignments and percent identity calculations can be determined using the BLAST program using its standard, default parameters. For nucleotide sequence alignment and sequence identity calculations, the BLASTN program is used with its default parameters (Gap opening penalty=5, Gap extension penalty=2, Nucleic match=2, Nucleic mismatch=−3, Expectation value=10.0, Word size=11, Max matches in a query range=0). For polypeptide sequence alignment and sequence identity calculations, BLASTP program is used with its default parameters (Alignment matrix=BLOSUM62; Gap costs: Existence=11, Extension=1; Compositional adjustments=Conditional compositional score, matrix adjustment; Expectation value=10.0; Word size=6; Max matches in a query range=0). Alternatively, the following program and parameters can be used: Align Plus software of Clone Manager Suite, version 5 (Sci-Ed Software); DNA comparison: Global comparison, Standard Linear Scoring matrix, Mismatch penalty=2, Open gap penalty=4, Extend gap penalty=1. Amino acid comparison: Global comparison, BLOSUM 62 Scoring matrix. In the embodiments described herein, the sequence identity is calculated using BLASTN or BLASTP programs using their default parameters. In the embodiments described herein, the sequence alignment of two or more sequences are performed using Clustal W using the suggested default parameters (Dealign input sequences: no; Mbed-like clustering guide-tree: yes; Mbed-like clustering iteration: yes; number of combined iterations: default(0); Max guide tree iterations: default; Max HMM iterations: default; Order: input).

### 6.2 ABC-Transporter, Nucleic Acids, Expression Cassettes, and Host Cells

In one aspect, provided herein are recombinant nucleic acids which express ABC-transporters. ABC-transporters of the invention can be identified by sequence-based searches against the sequences of known ABC-transporters. An exemplary sequence database of known ABC-transporters is provided by (Kovalchuk and Driessen, Phylogenetic Analysis of Fungal ABC Transporters, BMC Genomics, 2010, 11:177). ABC-transporter BLAST databases may also be generated from additional organisms. In preferred embodiments, fungal sequence databases from (1) Hansenula polymorpha DL-1 (NRRL-Y-7560), (2) Yarrowia lipolytica ATCC 18945, (3) Arxula adeninivorans ATCC 76597, (4) S. cerevisiae CAT-1, (5) Lipomyces starkeyi ATCC 58690, (6)Kluyveromyces marxianus, (7) Kluyveromyces marxianus DMKU3-1042, (8) Komagataella phaffii NRRL Y-11430, (9) S. cerevisiae MBG3370, (10) S. cerevisiae MBG3373, (11) K. lactis ATCC 8585, (12) Candida utilis ATCC 22023, (13) Pichia pastoris ATCC 28485, and (14) Aspergillus oryzae NRRL5590 serve as sources of ABC-transporters of the invention.

Nucleotide ORF sequences generated from de novo genomic sequencing, assembly, and annotation of various organisms are analyzed by the tblastn algorithm using Biopython or any other suitable sequence analysis software. The tblastn algorithm provides alignments of protein sequences of known ABC-transporters with translated DNA of the nucleotide ORF sequences for each organism in all 6 possible reading frames using BLAST. Exemplary BLAST parameters are standard with evalue=1e-25 (Tables 4 and 5). Hits can be subsequently filtered to ensure a global alignment of at least 2000 nucleotides.

In other embodiments of the invention, the entire proteome of an organism can be pulled from Uniprot using the Uniprot API in order to create a database for a BLAST search. The blastp algorithm can be applied to the Uniprot derived database. In one embodiment, BLAST parameters can be standard, with evalue=0.001. In particular embodiments, filtering can be performed based on a percent identity cutoff of >40%, and a percent aligned length cutoff of ≥60%. In preferred embodiments, hits have to match at least one of the 610 seed sequences from the reference.

Once nucleotide sequences are identified, primers can be designed to amplify each complete ORF amplified via PCR. Each PCR primer should ideally have flanking homology to the promoter and terminator DNA sequences of a promoter and terminator used in a heterologous nucleotide expression cassette added to the ends to facilitate homologous recombination of the amplified gene into a landing pad target site to produce the specific ABC-transporter expression cassette. Each ABC-transporter gene can be transformed individually as a single copy into the parental Reb M yeast strain described herein and screened for the ability to increase product titers when overexpressed in vivo.

In certain embodiments the recombinant nucleic acids encode a polypeptide that has the amino acid sequence provided in any of SEQ ID NOS: 1-8. In certain embodiments, the recombinant nucleic acid contains the nucleotide sequence provided in any of SEQ ID NOS: 20-27.

Also provided herein are host cells comprising one or more of the ABC-transporter polypeptides or nucleic acids provided herein that are capable of producing steviol glycosides. In certain embodiments, the host cells can produce steviol glycosides from a carbon source in a culture medium. In particular embodiments, the host cells can produce steviol from a carbon source in a culture medium and can further produce Reb A or Reb D from the steviol. In particular embodiments, the host cells can further produce Reb M from the Reb D. In particular embodiments, the Reb D and/or Reb M is transported to the lumen of one or more organelles. In particular embodiments, the Reb D and/or Reb M is transported to the extracellular space (i.e., supernatant).

In certain embodiments, host cells expressing ABC-transporters according to the above embodiments produce at least 20%, at least 30%, at least 40%, at least 50%, at least 60%, at least 70%, at least 80%, at least 90%, or at least 100% more total steviol glycoside (TSG) compared to the parent host cell lacking the ABC-transporter expression cassette.

In certain embodiments, host cells expressing ABC-transporters according to the above embodiments produce at least 20%, at least 30%, at least 40%, at least 50%, at least 60%, at least 70%, or at least 75% more TSG in the supernatant compared to the parent host cell lacking the ABC-transporter expression cassette. In a particular embodiment, host cells expressing ABC-transporters according to the above embodiments produce at least 2-fold, at least 3-fold, at least 4-fold, or at least 5-fold more TSG in the supernatant compared to the parent host cell lacking the ABC-transporter expression cassette.

In advantageous embodiments, the host cell can comprise one or more enzymatic pathways capable of making kaurenoic acid, said pathways taken individually or together. As described herein, the host cells comprise a Stevia rebaudiana kaurenoic acid hydroxylase provided herein, capable of converting kaurenoic acid to steviol. In certain embodiments, the host cell further comprises one or more enzymes capable of converting famesyl diphosphate to geranylgeranyl diphosphate. In certain embodiments, the host cell further comprises one or more enzymes capable of converting geranylgeranyl diphosphate to copalyl diphosphate. In certain embodiments, the host cell further comprises one or more enzymes capable of converting copalyl diphosphate to kaurene. In certain embodiments, the host cell further comprises one or more enzymes capable of converting kaurene to kaurenoic acid. In certain embodiments, the host cell further comprises one or more enzymes capable of converting steviol to one or more steviol glycosides. In certain embodiments, the host cell further comprises one, two, three, four, or more enzymes together capable of converting steviol to Reb A. In certain embodiments, the host cell further comprises one or more enzymes capable of converting Reb A to Reb D. In certain embodiments, the host cell further comprises one or more enzymes capable of converting Reb D to Reb M. Useful enzymes and nucleic acids encoding the enzymes are known to those of skill. Particularly useful enzymes and nucleic acids are described in the sections below and further described, for example, in US 2014/0329281 A1, US 2014/0357588 A1, US 2015/0159188, WO 2016/038095 A2, and US 2016/0198748 A1.

In further embodiments, the host cells further comprise one or more enzymes capable of making geranylgeranyl diphosphate from a carbon source. These include enzymes of the DXP pathway and enzymes of the MEV pathway. Useful enzymes and nucleic acids encoding the enzymes are known to those of skill in the art. Exemplary enzymes of each pathway are described below and further described, for example, in US 2016/0177341 A1 which is incorporated herein by reference in its entirety.

In some embodiments, the host cells comprise one or more or all of the isoprenoid pathway enzymes selected from the group consisting of: (a) an enzyme that condenses two molecules of acetyl-coenzyme A to form acetoacetyl-CoA (e.g., an acetyl-coA thiolase); (b) an enzyme that condenses acetoacetyl-CoA with another molecule of acetyl-CoA to form 3-hydroxy-3-methylglutaryl-CoA (HMG-CoA) (e.g., an HMG-CoA synthase); (c) an enzyme that converts HMG-CoA into mevalonate (e.g., an HMG-CoA reductase); (d) an enzyme that converts mevalonate into mevalonate 5-phosphate (e.g., a mevalonate kinase); (e) an enzyme that converts mevalonate 5-phosphate into mevalonate 5-pyrophosphate (e.g., a phosphomevalonate kinase); (f) an enzyme that converts mevalonate 5-pyrophosphate into isopentenyl diphosphate (IPP) (e.g., a mevalonate pyrophosphate decarboxylase); (g) an enzyme that converts IPP into dimethylallyl pyrophosphate (DMAPP) (e.g., an IPP isomerase); (h) a polyprenyl synthase that can condense IPP and/or DMAPP molecules to form polyprenyl compounds containing more than five carbons; (i) an enzyme that condenses IPP with DMAPP to form geranyl pyrophosphate (GPP) (e.g., a GPP synthase); (j) an enzyme that condenses two molecules of IPP with one molecule of DMAPP (e.g., an FPP synthase); (k) an enzyme that condenses IPP with GPP to form famesyl pyrophosphate (FPP) (e.g., an FPP synthase); (1) an enzyme that condenses IPP and DMAPP to form geranylgeranyl pyrophosphate (GGPP); and (m) an enzyme that condenses IPP and FPP to form GGPP.

In certain embodiments, the additional enzymes are native. In advantageous embodiments, the additional enzymes are heterologous. In certain embodiments, two or more enzymes can be combined in one polypeptide.

### 6.3 Cell Strains

Host cells useful compositions and methods provided herein include archae, prokaryotic, or eukaryotic cells.

Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Streptomyces, Synechococcus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. In a particular embodiment, the host cell is an Escherichia coli cell.

Suitable archae hosts include, but are not limited to, cells belonging to the genera: Aeropyrum, Archaeoglobus, Halobacterium, Methanococcus, Methanobacterium, Pyrococcus, Sulfolobus, and Thermoplasma. Examples of archae strains include, but are not limited to: Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

Suitable eukaryotic hosts include, but are not limited to, fungal cells, algal cells, insect cells, and plant cells. In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the host microbe is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorpha (now known as Pichia angusta). In some embodiments, the host microbe is a strain of the genus Candida, such as Candida lipolytica, Candida guilliermondii, Candida krusei, Candida pseudotropicalis, or Candida utilis.

In a particular embodiment, the host microbe is Saccharomyces cerevisiae. In some embodiments, the host is a strain of Saccharomyces cerevisiae selected from the group consisting of Baker's yeast, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the host microbe is a strain of Saccharomyces cerevisiae selected from the group consisting of PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

In some embodiments, the host microbe is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

### 6.4 The Steviol and Steviol Glycoside Biosynthesis Pathways

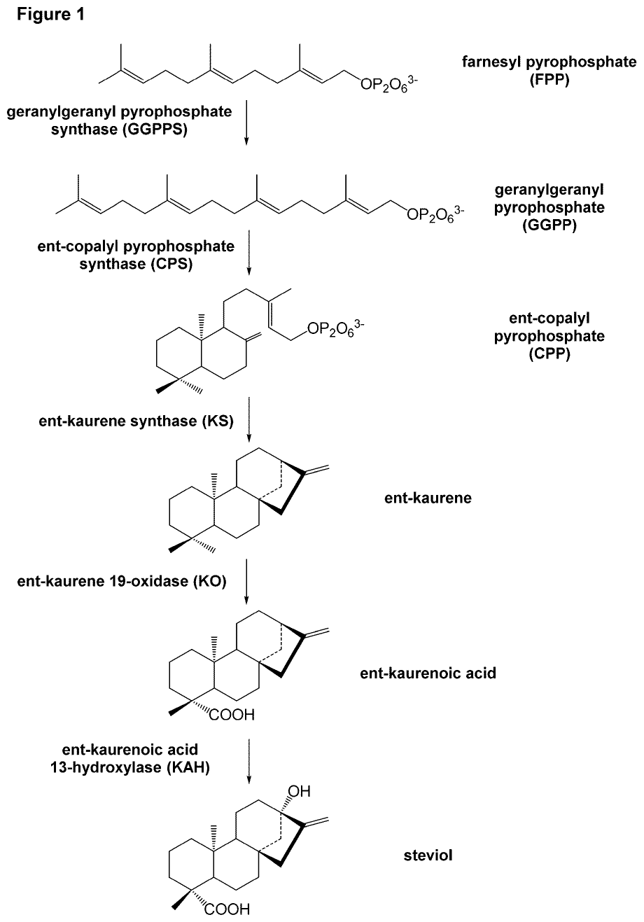

In some embodiments, a steviol biosynthesis pathway and/or a steviol glycoside biosynthesis pathway is activated in the genetically modified host cells provided herein by engineering the cells to express polynucleotides and/or polypeptides encoding one or more enzymes of the pathway. FIG. 1 illustrates an exemplary steviol biosynthesis pathway.

Thus, in some embodiments, the genetically modified host cells provided herein comprise a heterologous polynucleotide encoding a polypeptide having geranylgeranyl diphosphate synthase (GGPPS) activity. In some embodiments, the genetically modified host cells provided herein comprise a heterologous polynucleotide encoding a polypeptide having copalyl diphosphate synthase or ent-copalyl pyrophosphate synthase (CDPS; also referred to as ent-copalyl pyrophosphate synthase or CPS) activity. In some embodiments, the genetically modified host cells provided herein comprise a heterologous polynucleotide encoding a polypeptide having kaurene synthase (KS; also referred to as ent-kaurene synthase) activity. In particular embodiments, the genetically modified host cells provided herein comprise a heterologous polynucleotide encoding a polypeptide having kaurene oxidase activity (KO; also referred to as ent-kaurene 19-oxidase) as described herein. In particular embodiments, the genetically modified host cells provided herein comprise a heterologous polynucleotide encoding a polypeptide having kaurenoic acid hydroxylase polypeptide activity (KAH; also referred to as steviol synthase) according to the embodiments provided herein. In some embodiments, the genetically modified host cells provided herein comprise a heterologous polynucleotide encoding a polypeptide having cytochrome P450 reductase (CPR) activity.

In some embodiments, the genetically modified host cells provided herein comprise a heterologous polynucleotide encoding a polypeptide having UGT74G1 activity. In some embodiments, the genetically modified host cells provided herein comprise a heterologous polynucleotide encoding a polypeptide having UGT76G1 activity. In some embodiments, the genetically modified host cells provided herein comprise a heterologous polynucleotide encoding a polypeptide having UGT85C2 activity. In some embodiments, the genetically modified host cells provided herein comprise a heterologous polynucleotide encoding a polypeptide having UGT91D activity. In some embodiments, the genetically modified host cells provided herein comprise a heterologous polynucleotide encoding a polypeptide having UGT_{AD} activity. As described below, UGT_{AD} refers to a uridine diphosphate-dependent glycosyl transferase capable of transferring a glucose moiety to the C-2′ position of the 19-O-glucose of Reb A to produce Reb D.

In certain embodiments, the host cell comprises a variant enzyme. In certain embodiments, the variant can comprise up to 15, 10, 9, 8, 7, 6, 5, 4, 3, 2, or 1 amino acid substitutions relative to the relevant polypeptide. In certain embodiments, the variant can comprise up to 15, 10, 9, 8, 7, 6, 5, 4, 3, 2, or 1 conservative amino acid substitutions relative to the reference polypeptide. In certain embodiments, any of the nucleic acids described herein can be optimized for the host cell, for instance codon optimized.

Exemplary nucleic acids and enzymes of a steviol biosynthesis pathway and/or a steviol glycoside biosynthesis pathway are described below.

### 6.4.1 Geranylgeranyl Diphosphate Synthase (GGPPS)

Geranylgeranyl diphosphate synthases (EC 2.5.1.29) catalyze the conversion of famesyl pyrophosphate into geranylgeranyl diphosphate. Illustrative examples of enzymes include those of Stevia rebaudiana (accession no. ABD92926), Gibberella fujikuroi (accession no. CAA75568), Mus musculus (accession no. AAH69913), Thalassiosira pseudonana (accession no. XP_002288339), Streptomyces clavuligerus (accession no. ZP_05004570), Sulfulobus acidocaldarius (accession no. BAA43200), Synechococcus sp. (accession no. ABC98596), Arabidopsis thaliana (accession no. NP_195399), and Blakeslea trispora (accession no. AFC92798.1), and those described in US 2014/0329281 A1. Nucleic acids encoding these enzymes are useful in the cells and methods provided herein. In certain embodiments, provided herein are cells and methods using a nucleic acid having at least 80%, 85%, 90%, or 95% sequence identity to at least one of these GGPPS nucleic acids. In certain embodiments, provided herein are cells and methods using a nucleic acid that encodes a polypeptide having at least 80%, 85%, 90%, 95% sequence identity to at least one of these GGPPS enzymes.

### 6.4.2 Copalyl Diphosphate Synthase (CDPS)

Copalyl diphosphate synthases (EC 5.5.1.13) catalyze the conversion of geranylgeranyl diphosphate into copalyl diphosphate. Illustrative examples of enzymes include those of Stevia rebaudiana (accession no. AAB87091), Streptomyces clavuligerus (accession no. EDY51667), Bradyrhizobium japonicum (accession no. AAC28895.1), Zea mays (accession no. AY562490), Arabidopsis thaliana (accession no. NM1 16512), and Oryza sativa (accession no. Q5MQ85.1), and those described in US 2014/0329281 A1. Nucleic acids encoding these enzymes are useful in the cells and methods provided herein. In certain embodiments, provided herein are cells and methods using a nucleic acid having at least 80%, 85%, 90%, or 95% sequence identity to at least one of these CDPS nucleic acids. In certain embodiments, provided herein are cells and methods using a nucleic acid that encodes a polypeptide having at least 80%, 95%, 90%, or 95% sequence identity to at least one of these CDPS enzymes.

### 6.4.3 Kaurene Synthase (KS)

Kaurene synthases (EC 4.2.3.19) catalyze the conversion of copalyl diphosphate into kaurene and diphosphate. Illustrative examples of enzymes include those of Bradyrhizobium japonicum (accession no. AAC28895.1), Phaeosphaeria sp. (accession no. 013284), Arabidopsis thaliana (accession no. Q9SAK2), and Picea glauca (accession no. ADB55711.1), and those described in US 2014/0329281 A1. Nucleic acids encoding these enzymes are useful in the cells and methods provided herein. In certain embodiments, provided herein are cells and methods using a nucleic acid having at least 80%, 85%, 90%, or 95% sequence identity to at least one of these KS nucleic acids. In certain embodiments, provided herein are cells and methods using a nucleic acid that encodes a polypeptide having at least 80%, 85%, 85%, 90%, or 95% sequence identity to at least one of these KS enzymes.

### 6.4.4 Bifunctional Copalyl Diphosphate Synthase (CDPS) and Kaurene Synthase (KS)

CDPS-KS bifunctional enzymes (EC 5.5.1.13 and EC 4.2.3.19) also can be used. Illustrative examples of enzymes include those of Phomopsis amygdali (accession no. BAG30962), Physcomitrella patens (accession no. BAF61135), and Gibberella fujikuroi (accession no. Q9UVY5.1), and those described in US 2014/0329281 A1, US 2014/0357588 A1, US 2015/0159188, and WO 2016/038095 A2. Nucleic acids encoding these enzymes are useful in the cells and methods provided herein. In certain embodiments, provided herein are cells and methods using a nucleic acid having at least 80%, 85%, 90%, or 95% sequence identity to at least one of these CDPS-KS nucleic acids. In certain embodiments, provided herein are cells and methods using a nucleic acid that encodes a polypeptide having at least 80%, 85%, 90%, or 95% sequence identity to at least one of these CDPS-KS enzymes.

### 6.4.5 Ent-Kaurene Oxidase (KO)

Ent-kaurene oxidases (EC 1.14.13.78; also referred to as kaurene oxidases herein) catalyze the conversion of kaurene into kaurenoic acid. Illustrative examples of enzymes include those of Oryza sativa (accession no. Q5Z5R4), Gibberella fujikuroi (accession no. 094142), Arabidopsis thaliana (accession no. Q93ZB2), Stevia rebaudiana (accession no. AAQ63464.1), and Pisum sativum (Uniprot no. Q6XAF4), and those described in US 2014/0329281 A1, US 2014/0357588 A1, US 2015/0159188, and WO 2016/038095 A2. Nucleic acids encoding these enzymes are useful in the cells and methods provided herein. In certain embodiments, provided herein are cells and methods using a nucleic acid having at least 80%, 85%, 90%, or 95% sequence identity to at least one of these KO nucleic acids. In certain embodiments, provided herein are cells and methods using a nucleic acid that encodes a polypeptide having at least 80%, 85%, 90%, or 95% sequence identity to at least one of these KO enzymes.

### 6.4.6 Steviol Synthase (KAH)

Steviol synthases, or kaurenoic acid hydroxylases (KAH), (EC 1.14.13) catalyze the conversion of kaurenoic acid into steviol. Illustrative examples of enzymes include those of Stevia rebaudiana (accession no. ACD93722), Stevia rebaudiana (SEQ ID NO:10) Arabidopsis thaliana (accession no. NP_197872), Vitis vinifera (accession no. XP_002282091), and Medicago trunculata (accession no. ABC59076), and those described in US 2014/0329281 A1, US 2014/0357588 A1, US 2015/0159188, and WO 2016/038095 A2. Nucleic acids encoding these enzymes are useful in the cells and methods provided herein. In certain embodiments, provided herein are cells and methods using a nucleic acid having at least 80%, 85%, 90%, or 95% sequence identity to at least one of these KAH nucleic acids. In certain embodiments, provided herein are cells and methods using a nucleic acid that encodes a polypeptide having at least 80%, 85%, 90%, or 95% sequence identity to at least one of these KAH enzymes.

### 6.4.7 Cytochrome P450 Reductase (CPR)

Cytochrome P450 reductases (EC 1.6.2.4) are necessary for the activity of KO and/or KAH above. Illustrative examples of enzymes include those of Stevia rebaudiana (accession no. ABB88839) Arabidopsis thaliana (accession no. NP_194183), Gibberella fujikuroi (accession no. CAE09055), and Artemisia annua (accession no. ABC47946.1), and those described in US 2014/0329281 A1, US 2014/0357588 A1, US 2015/0159188, and WO 2016/038095 A2. Nucleic acids encoding these enzymes are useful in the cells and methods provided herein. In certain embodiments, provided herein are cells and methods using a nucleic acid having at least 80%, 85%, 90%, or 95% sequence identity to at least one of these CPR nucleic acids. In certain embodiments, provided herein are cells and methods using a nucleic acid that encodes a polypeptide having at least 80%, 85%, 90%, or 95% sequence identity to at least one of these CPR enzymes.

### 6.4.8 UDP Glycosyltransferase 74G1 (UGT74G1)

A UGT74G1 is capable of functioning as a uridine 5′-diphospho glucosyl: steviol 19-COOH transferase and as a uridine 5′-diphospho glucosyl: steviol-13-O-glucoside 19-COOH transferase. As shown in FIG. 1, a UGT74G1 is capable of converting steviol to 19-glycoside. A UGT74G1 is also capable of converting steviolmonoside to rubusoside. A UGT74G1 may be also capable of converting steviolbioside to stevioside. Illustrative examples of enzymes include those of Stevia rebaudiana (e.g., those of Richman et al., 2005, Plant J. 41: 56-67 and US 2014/0329281 and WO 2016/038095 A2 and accession no. AAR06920.1). Nucleic acids encoding these enzymes are useful in the cells and methods provided herein. In certain embodiments, provided herein are cells and methods using a nucleic acid having at least 80%, 85%, 90%, or 95% sequence identity to at least one of these UGT74G1 nucleic acids. In certain embodiments, provided herein are cells and methods using a nucleic acid that encodes a polypeptide having at least 80%, 85%, 90%, or 95% sequence identity to at least one of these UGT74G1 enzymes.

### 6.4.9 UDP Glycosyltransferase 76G1 (UGT76G1)

A UGT76G1 is capable of transferring a glucose moiety to the C-3′ of the C-13-0-glucose of the acceptor molecule, a steviol 1,2 glycoside. Thus, a UGT76G1 is capable of functioning as a uridine 5′-diphospho glucosyl: steviol 13-O-1,2 glucoside C-3′ glucosyl transferase and a uridine 5′-diphospho glucosyl: steviol-19-O-glucose, 13-O-1,2 bioside C-3′ glucosyl transferase. UGT76G1 is capable of converting steviolbioside to Reb B. A UGT76G1 is also capable of converting stevioside to Reb A. A UGT76G1 is also capable of converting Reb D to Reb M. Illustrative examples of enzymes include those of Stevia rebaudiana (e.g., those of Richman et al., 2005, Plant J. 41: 56-67 and US 2014/0329281 A1 and WO 2016/038095 A2 and accession no. AAR06912.1). Nucleic acids encoding these enzymes are useful in the cells and methods provided herein. In certain embodiments, provided herein are cells and methods using a nucleic acid having at least 80%, 85%, 90%, or 95% sequence identity to at least one of these UGT76G1 nucleic acids. In certain embodiments, provided herein are cells and methods using a nucleic acid that encodes a polypeptide having at least 80%, 85%, 90%, or 95% sequence identity to at least one of these UGT76G1 enzymes.

### 6.4.10 UDP Glycosyltransferase 85C2 (UGT85C2)

A UGT85C2 is capable of functioning as a uridine 5′-diphospho glucosyl:steviol 13-OH transferase, and a uridine 5′-diphospho glucosyl:steviol-19-O-glucoside 13-OH transferase. A UGT85C2 is capable of converting steviol to steviolmonoside, and is also capable of converting 19-glycoside to rubusoside. Illustrative examples of enzymes include those of Stevia rebaudiana (e.g., those of Richman et al., 2005, Plant J. 41: 56-67 and US 2014/0329281 A1 and WO 2016/038095 A2 and accession no. AAR06916.1). Nucleic acids encoding these enzymes are useful in the cells and methods provided herein. In certain embodiments, provided herein are cells and methods using a nucleic acid having at least 80%, 85%, 90%, or 95% sequence identity to at least one of these UGT85C2 nucleic acids. In certain embodiments, provided herein are cells and methods using a nucleic acid that encodes a polypeptide having at least 80%, 85%, 90%, or 95% sequence identity to at least one of these UGT85C2 enzymes.

### 6.4.11 UDP-Glycosyltransferase 91D (UGT91D)

A UGT91D is capable of functioning as a uridine 5′-diphosphoglucosyl:steviol-13-O-glucoside transferase, transferring a glucose moiety to the C-2′ of the 13-O-glucose of the acceptor molecule, steviol-13-O-glucoside (steviolmonoside) to produce steviolbioside. A UGT91D is also capable of functioning as a uridine 5′-diphosphoglucosyl:rubusoside transferase, transferring a glucose moiety to the C-2′ of the 13-O-glucose of the acceptor molecule, rubusoside, to provide stevioside. A UGT91D is also referred to as UGT91D2, UGT91D2e, or UGT91D-like3. Illustrative examples of UGT91D enzymes include those of Stevia rebaudiana (e.g., those of UGT sequence with accession no. ACE87855.1, US 2014/0329281 A1, WO 2016/038095 A2, and SEQ ID NO:7). Nucleic acids encoding these enzymes are useful in the cells and methods provided herein. In certain embodiments, provided herein are cells and methods using a nucleic acid having at least 80%, 85%, 90%, or 95% sequence identity to at least one of these UGT91D nucleic acids. In certain embodiments, provided herein are cells and methods using a nucleic acid that encodes a polypeptide having at least 80%, 85%, 90%, or 95% sequence identity to at least one of these UGT91D enzymes.

### 6.4.12 Uridine Diphosphate-Dependent Glycosyl Transferase Capable of Converting Reb a to Reb D (UGT_{AD})

A uridine diphosphate-dependent glycosyl transferase (UGT_{AD}) is capable of transferring a glucose moiety to the C-2′ position of the 19-O-glucose of Reb A to produce Reb D. A UGT_{AD} is also capable of transferring a glucose moiety to the C-2′ position of the 19-O-glucose of stevioside to produce Reb E. Useful examples of UGTs include Os_UGT_91C1 from Oryza sativa (also referred to as EUGT11 in Houghton-Larsen et al., WO 2013/022989 A2; XP_015629141.1) and Sl_UGT_101249881 from Solanum lycopersicum (also referred to as UGTSL2 in Markosyan et al., WO2014/193888 A1; XP_004250485.1). Further useful UGTs include UGT40087 (XP_004982059.1; as described in WO 2018/031955), sr.UGT_9252778, Bd_UGT10840 (XP_003560669.1), Hv_UGT V1 (BAJ94055.1), Bd_UGT10850 (XP_010230871.1), and Ob_UGT91B1 like (XP_006650455.1). Any UGT or UGT variant can be used in the compositions and methods described herein. Nucleic acids encoding these enzymes are useful in the cells and methods provided herein. In certain embodiments, provided herein are cells and methods using a nucleic acid having at least 80%, 85%, 90%, or 95% sequence identity to at least one of the UGTs. In certain embodiments, provided herein are cells and methods using a nucleic acid that encodes a polypeptide having at least 80%, 85%, 90%, or 95% sequence identity to at least one of these UGTs. In certain embodiments, provided herein are a nucleic acid that encodes a UGT variant described herein.

6.5 MEV Pathway FPP and/or GGPP Production

In some embodiments, a genetically modified host cell provided herein comprises one or more heterologous enzymes of the MEV pathway, useful for the formation of FPP and/or GGPP. In some embodiments, the one or more enzymes of the MEV pathway comprise an enzyme that condenses acetyl-CoA with malonyl-CoA to form acetoacetyl-CoA. In some embodiments, the one or more enzymes of the MEV pathway comprise an enzyme that condenses two molecules of acetyl-CoA to form acetoacetyl-CoA. In some embodiments, the one or more enzymes of the MEV pathway comprise an enzyme that condenses acetoacetyl-CoA with acetyl-CoA to form HMG-CoA. In some embodiments, the one or more enzymes of the MEV pathway comprise an enzyme that converts HMG-CoA to mevalonate. In some embodiments, the one or more enzymes of the MEV pathway comprise an enzyme that phosphorylates mevalonate to mevalonate 5-phosphate. In some embodiments, the one or more enzymes of the MEV pathway comprise an enzyme that converts mevalonate 5-phosphate to mevalonate 5-pyrophosphate. In some embodiments, the one or more enzymes of the MEV pathway comprise an enzyme that converts mevalonate 5-pyrophosphate to isopentenyl pyrophosphate. In some embodiments, the one or more enzymes of the MEV pathway comprise an enzyme that converts isopentenyl pyrophosphate to dimethylallyl diphosphate.

In some embodiments, the one or more enzymes of the MEV pathway are selected from the group consisting of acetyl-CoA thiolase, acetoacetyl-CoA synthetase, HMG-CoA synthase, HMG-CoA reductase, mevalonate kinase, phosphomevalonate kinase, mevalonate pyrophosphate decarboxylase, and isopentyl diphosphate:dimethylallyl diphosphate isomerase (IDI or IPP isomerase). In some embodiments, with regard to the enzyme of the MEV pathway capable of catalyzing the formation of acetoacetyl-CoA, the genetically modified host cell comprises either an enzyme that condenses two molecules of acetyl-CoA to form acetoacetyl-CoA, e.g., acetyl-CoA thiolase; or an enzyme that condenses acetyl-CoA with malonyl-CoA to form acetoacetyl-CoA, e.g., acetoacetyl-CoA synthase. In some embodiments, the genetically modified host cell comprises both an enzyme that condenses two molecules of acetyl-CoA to form acetoacetyl-CoA, e.g., acetyl-CoA thiolase; and an enzyme that condenses acetyl-CoA with malonyl-CoA to form acetoacetyl-CoA, e.g., acetoacetyl-CoA synthase.

In some embodiments, the host cell comprises one or more heterologous nucleotide sequences encoding more than one enzyme of the MEV pathway. In some embodiments, the host cell comprises one or more heterologous nucleotide sequences encoding two enzymes of the MEV pathway. In some embodiments, the host cell comprises one or more heterologous nucleotide sequences encoding an enzyme that can convert HMG-CoA into mevalonate and an enzyme that can convert mevalonate into mevalonate 5-phosphate. In some embodiments, the host cell comprises one or more heterologous nucleotide sequences encoding three enzymes of the MEV pathway. In some embodiments, the host cell comprises one or more heterologous nucleotide sequences encoding four enzymes of the MEV pathway. In some embodiments, the host cell comprises one or more heterologous nucleotide sequences encoding five enzymes of the MEV pathway. In some embodiments, the host cell comprises one or more heterologous nucleotide sequences encoding six enzymes of the MEV pathway. In some embodiments, the host cell comprises one or more heterologous nucleotide sequences encoding seven enzymes of the MEV pathway. In some embodiments, the host cell comprises a plurality of heterologous nucleic acids encoding all of the enzymes of the MEV pathway.

In some embodiments, the genetically modified host cell further comprises a heterologous nucleic acid encoding an enzyme that can convert isopentenyl pyrophosphate (IPP) into dimethylallyl pyrophosphate (DMAPP). In some embodiments, the genetically modified host cell further comprises a heterologous nucleic acid encoding an enzyme that can condense IPP and/or DMAPP molecules to form a polyprenyl compound. In some embodiments, the genetically modified host cell further comprise a heterologous nucleic acid encoding an enzyme that can modify IPP or a polyprenyl to form an isoprenoid compound such as FPP.

### 6.5.1 Conversion of Acetyl-CoA to Acetoacetyl-CoA

In some embodiments, the genetically modified host cell comprises a heterologous nucleotide sequence encoding an enzyme that can condense two molecules of acetyl-coenzyme A to form acetoacetyl-CoA, e.g., an acetyl-CoA thiolase. Illustrative examples of nucleotide sequences encoding such an enzyme include, but are not limited to: (NC_000913 REGION: 2324131.2325315; Escherichia coli), (D49362; Paracoccus denitrificans), and (L20428; Saccharomyces cerevisiae).

Acetyl-CoA thiolase catalyzes the reversible condensation of two molecules of acetyl-CoA to yield acetoacetyl-CoA, but this reaction is thermodynamically unfavorable; acetoacetyl-CoA thiolysis is favored over acetoacetyl-CoA synthesis. Acetoacetyl-CoA synthase (AACS) (alternately referred to as acetyl-CoA:malonyl-CoA acyltransferase; EC 2.3.1.194) condenses acetyl-CoA with malonyl-CoA to form acetoacetyl-CoA. In contrast to acetyl-CoA thiolase, AACS-catalyzed acetoacetyl-CoA synthesis is essentially an energy-favored reaction, due to the associated decarboxylation of malonyl-CoA. In addition, AACS exhibits no thiolysis activity against acetoacetyl-CoA, and thus the reaction is irreversible.

In host cells comprising acetyl-CoA thiolase and a heterologous ADA and/or phosphotransacetylase (PTA), the reversible reaction catalyzed by acetyl-CoA thiolase, which favors acetoacetyl-CoA thiolysis, may result in a large acetyl-CoA pool. In view of the reversible activity of ADA, this acetyl-CoA pool may in turn drive ADA towards the reverse reaction of converting acetyl-CoA to acetaldehyde, thereby diminishing the benefits provided by ADA towards acetyl-CoA production. Similarly, the activity of PTA is reversible, and thus, a large acetyl-CoA pool may drive PTA towards the reverse reaction of converting acetyl-CoA to acetyl phosphate. Therefore, in some embodiments, in order to provide a strong pull on acetyl-CoA to drive the forward reaction of ADA and PTA, the MEV pathway of the genetically modified host cell provided herein utilizes an acetoacetyl-CoA synthase to form acetoacetyl-CoA from acetyl-CoA and malonyl-CoA.

In some embodiments, the AACS is from Streptomyces sp. strain CL190 (Okamura et al., Proc Natl Acad Sci USA 107(25):11265-70 (2010). Representative AACS nucleotide sequences of Streptomyces sp. strain CL190 include accession number AB540131.1. Representative AACS protein sequences of Streptomyces sp. strain CL190 include accession numbers D7URV0, BAJ10048. Other acetoacetyl-CoA synthases useful for the compositions and methods provided herein include, but are not limited to, Streptomyces sp. (AB183750; KO-3988 BAD86806); S. anulatus strain 9663 (FN178498; CAX48662); Streptomyces sp. KO-3988 (AB212624; BAE78983); Actinoplanes sp. A40644 (AB113568; BAD07381); Streptomyces sp. C (NZ_ACEW010000640; ZP_05511702); Nocardiopsis dassonvillei DSM 43111 (NZ_ABUI01000023; ZP_04335288); Mycobacterium ulcerans Agy99 (NC_008611; YP_907152); Mycobacterium marinum M (NC_010612; YP_001851502); Streptomyces sp. Mgl (NZ_DS570501; ZP_05002626); Streptomyces sp. AA4 (NZ_ACEV01000037; ZP_05478992); S. roseosporus NRRL 15998 (NZ_ABYB01000295; ZP_04696763); Streptomyces sp. ACTE (NZ_ADFD01000030; ZP_06275834); S. viridochromogenes DSM 40736 (NZ_ACEZ01000031; ZP_05529691); Frankia sp. CcI3 (NC 007777; YP_480101); Nocardia brasiliensis (NC_018681; YP_006812440.1); and Austwickia chelonae (NZ_BAGZ01000005; ZP_10950493.1). Additional suitable acetoacetyl-CoA synthases include those described in U.S. Patent Application Publication Nos. 2010/0285549 and 2011/0281315, the contents of which are incorporated by reference in their entireties.

Acetoacetyl-CoA synthases also useful in the compositions and methods provided herein include those molecules which are said to be “derivatives” of any of the acetoacetyl-CoA synthases described herein. Such a “derivative” has the following characteristics: (1) it shares substantial homology with any of the acetoacetyl-CoA synthases described herein; and (2) is capable of catalyzing the irreversible condensation of acetyl-CoA with malonyl-CoA to form acetoacetyl-CoA. A derivative of an acetoacetyl-CoA synthase is said to share “substantial homology” with acetoacetyl-CoA synthase if the amino acid sequences of the derivative is at least 80%, and more preferably at least 90%, and most preferably at least 95%, the same as that of acetoacetyl-CoA synthase.

### 6.5.2 Conversion of Acetoacetyl-CoA to HMG-CoA

In some embodiments, the host cell comprises a heterologous nucleotide sequence encoding an enzyme that can condense acetoacetyl-CoA with another molecule of acetyl-CoA to form 3-hydroxy-3-methylglutaryl-CoA (HMG-CoA), e.g., a HMG-CoA synthase. Illustrative examples of nucleotide sequences encoding such an enzyme include, but are not limited to: (NC_001145. complement 19061.20536; Saccharomyces cerevisiae), (X96617; Saccharomyces cerevisiae), (X83882; Arabidopsis thaliana), (AB037907; Kitasatospora griseola), (BT007302; Homo sapiens), and (NC_002758, Locus tag SAV2546, GeneID 1122571; Staphylococcus aureus).

### 6.5.3 Conversion of HMG-CoA to Mevalonate

In some embodiments, the host cell comprises a heterologous nucleotide sequence encoding an enzyme that can convert HMG-CoA into mevalonate, e.g., a HMG-CoA reductase. In some embodiments, HMG-CoA reductase is an NADH-using hydroxymethylglutaryl-CoA reductase-CoA reductase. HMG-CoA reductases (EC 1.1.1.34; EC 1.1.1.88) catalyze the reductive deacylation of (S)-HMG-CoA to (R)-mevalonate, and can be categorized into two classes, class I and class II HMGrs. Class I includes the enzymes from eukaryotes and most archaea, and class II includes the HMG-CoA reductases of certain prokaryotes and archaea. In addition to the divergence in the sequences, the enzymes of the two classes also differ with regard to their cofactor specificity. Unlike the class I enzymes, which utilize NADPH exclusively, the class II HMG-CoA reductases vary in the ability to discriminate between NADPH and NADH. See, e.g., Hedl et al., Journal of Bacteriology 186 (7): 1927-1932 (2004). Co-factor specificities for select class II HMG-CoA reductases are provided below.

Co-factor specificities for select class II HMG-CoA reductases

| | Coenzyme | Kₘ^{NADPH} | Kₘ^{NADH} |
|---|---|---|---|
| Source | specificity | (μm) | (μm) |
| P. mevalonii | NADH | | 80 |
| A. fulgidus | NAD(P)H | 500 | 160 |
| S. aureus | NAD(P)H | 70 | 100 |
| E. faecalis | NADPH | 30 | |

Useful HMG-CoA reductases for the compositions and methods provided herein include HMG-CoA reductases that are capable of utilizing NADH as a cofactor, e.g., HMG-CoA reductase from P. mevalonii, A. fulgidus or S. aureus. In particular embodiments, the HMG-CoA reductase is capable of only utilizing NADH as a cofactor, e.g., HMG-CoA reductase from P. mevalonii, S. pomeroyi or D. acidovorans.

In some embodiments, the NADH-using HMG-CoA reductase is from Pseudomonas mevalonii. The sequence of the wild-type mvaA gene of Pseudomonas mevalonii, which encodes HMG-CoA reductase (EC 1.1.1.88), has been previously described. See Beach and Rodwell, J. Bacteriol. 171:2994-3001 (1989). Representative mvaA nucleotide sequences of Pseudomonas mevalonii include accession number M24015. Representative HMG-CoA reductase protein sequences of Pseudomonas mevalonii include accession numbers AAA25837, P13702, MVAA_PSEMV.

In some embodiments, the NADH-using HMG-CoA reductase is from Silicibacter pomeroyi. Representative HMG-CoA reductase nucleotide sequences of Silicibacter pomeroyi include accession number NC_006569.1. Representative HMG-CoA reductase protein sequences of Silicibacter pomeroyi include accession number YP_164994.

In some embodiments, the NADH-using HMG-CoA reductase is from Delftia acidovorans. A representative HMG-CoA reductase nucleotide sequences of Delftia acidovorans includes NC_010002 REGION: complement (319980 . . . 321269). Representative HMG-CoA reductase protein sequences of Delftia acidovorans include accession number YP_001561318.

In some embodiments, the NADH-using HMG-CoA reductases is from Solanum tuberosum (Crane et al., J. Plant Physiol. 159:1301-1307 (2002)).

NADH-using HMG-CoA reductases also useful in the compositions and methods provided herein include those molecules which are said to be “derivatives” of any of the NADH-using HMG-CoA reductases described herein, e.g., from P. mevalonii, S. pomeroyi and D. acidovorans. Such a “derivative” has the following characteristics: (1) it shares substantial homology with any of the NADH-using HMG-CoA reductases described herein; and (2) is capable of catalyzing the reductive deacylation of (S)-HMG-CoA to (R)-mevalonate while preferentially using NADH as a cofactor. A derivative of an NADH-using HMG-CoA reductase is said to share “substantial homology” with NADH-using HMG-CoA reductase if the amino acid sequences of the derivative is at least 80%, and more preferably at least 90%, and most preferably at least 95%, the same as that of NADH-using HMG-CoA reductase.

As used herein, the phrase “NADH-using” means that the NADH-using HMG-CoA reductase is selective for NADH over NADPH as a cofactor, for example, by demonstrating a higher specific activity for NADH than for NADPH. In some embodiments, selectivity for NADH as a cofactor is expressed as a k_{cat}^{(NADH)}/k_{cat}^{(NADPH)} ratio. In some embodiments, the NADH-using HMG-CoA reductase has a k_{cat}^{(NADH)}/k_{cat}^{(NADPH)} ratio of at least 5, 10, 15, 20, 25 or greater than 25. In some embodiments, the NADH-using HMG-CoA reductase uses NADH exclusively. For example, an NADH-using HMG-CoA reductase that uses NADH exclusively displays some activity with NADH supplied as the sole cofactor in vitro, and displays no detectable activity when NADPH is supplied as the sole cofactor. Any method for determining cofactor specificity known in the art can be utilized to identify HMG-CoA reductases having a preference for NADH as cofactor, including those described by Kim et al., Protein Science 9:1226-1234 (2000); and Wilding et al., J Bacteriol. 182(18):5147-52 (2000), the contents of which are hereby incorporated in their entireties.

In some embodiments, the NADH-using HMG-CoA reductase is engineered to be selective for NADH over NAPDH, for example, through site-directed mutagenesis of the cofactor-binding pocket. Methods for engineering NADH-selectivity are described in Watanabe et al., Microbiology 153:3044-3054 (2007), and methods for determining the cofactor specificity of HMG-CoA reductases are described in Kim et al., Protein Sci. 9:1226-1234 (2000), the contents of which are hereby incorporated by reference in their entireties.

In some embodiments, the NADH-using HMG-CoA reductase is derived from a host species that natively comprises a mevalonate degradative pathway, for example, a host species that catabolizes mevalonate as its sole carbon source. Within these embodiments, the NADH-using HMG-CoA reductase, which normally catalyzes the oxidative acylation of internalized (R)-mevalonate to (S)-HMG-CoA within its native host cell, is utilized to catalyze the reverse reaction, that is, the reductive deacylation of (S)-HMG-CoA to (R)-mevalonate, in a genetically modified host cell comprising a mevalonate biosynthetic pathway. Prokaryotes capable of growth on mevalonate as their sole carbon source have been described by: Anderson et al., J Bacteriol, 171(12):6468-6472 (1989); Beach et al., J Bacteriol. 171:2994-3001 (1989); Bensch et al., J Biol. Chem. 245:3755-3762; Fimongnari et al., Biochemistry 4:2086-2090 (1965); Siddiqi et al., Biochem. Biophys. Res. Commun. 8:110-113 (1962); Siddiqi et al., J. Bacteriol. 93:207-214 (1967); and Takatsuji et al., Biochem. Biophys. Res. Commun. 110:187-193 (1983), the contents of which are hereby incorporated by reference in their entireties.

In some embodiments of the compositions and methods provided herein, the host cell comprises both a NADH-using HMGr and an NADPH-using HMG-CoA reductase. Illustrative examples of nucleotide sequences encoding an NADPH-using HMG-CoA reductase include, but are not limited to: (NM_206548; Drosophila melanogaster), (NC_002758, Locus tag SAV2545, GeneID 1122570; Staphylococcus aureus), (AB015627; Streptomyces sp. KO 3988), (AX128213, providing the sequence encoding a truncated HMG-CoA reductase; Saccharomyces cerevisiae), and (NC_001145: complement (115734.118898; Saccharomyces cerevisiae).

### 6.5.4 Conversion of Mevalonate to Mevalonate-5-Phosphate

In some embodiments, the host cell comprises a heterologous nucleotide sequence encoding an enzyme that can convert mevalonate into mevalonate 5-phosphate, e.g., a mevalonate kinase. Illustrative examples of nucleotide sequences encoding such an enzyme include, but are not limited to: (L77688; Arabidopsis thaliana), and (X55875; Saccharomyces cerevisiae).

### 6.5.5 Conversion of Mevalonate-5-Phosphate to Mevalonate-5-Pyrophosphate

In some embodiments, the host cell comprises a heterologous nucleotide sequence encoding an enzyme that can convert mevalonate 5-phosphate into mevalonate 5-pyrophosphate, e.g., a phosphomevalonate kinase. Illustrative examples of nucleotide sequences encoding such an enzyme include, but are not limited to: (AF429385; Hevea brasiliensis), (NM_006556; Homo sapiens), and (NC_001145. complement 712315.713670; Saccharomyces cerevisiae).

### 6.5.6 Conversion of Mevalonate-5-Pyrophosphate to IPP

In some embodiments, the host cell comprises a heterologous nucleotide sequence encoding an enzyme that can convert mevalonate 5-pyrophosphate into isopentenyl diphosphate (IPP), e.g., a mevalonate pyrophosphate decarboxylase. Illustrative examples of nucleotide sequences encoding such an enzyme include, but are not limited to: (X97557; Saccharomyces cerevisiae), (AF290095; Enterococcus faecium), and (U49260; Homo sapiens).

### 6.5.7 Conversion of IPP to DMAPP

In some embodiments, the host cell further comprises a heterologous nucleotide sequence encoding an enzyme that can convert IPP generated via the MEV pathway into dimethylallyl pyrophosphate (DMAPP), e.g., an IPP isomerase. Illustrative examples of nucleotide sequences encoding such an enzyme include, but are not limited to: (NC_000913, 3031087.3031635; Escherichia coli), and (AF082326; Haematococcus pluvialis).

### 6.5.8 Polyprenyl Synthases

In some embodiments, the host cell further comprises a heterologous nucleotide sequence encoding a polyprenyl synthase that can condense IPP and/or DMAPP molecules to form polyprenyl compounds containing more than five carbons.

In some embodiments, the host cell comprises a heterologous nucleotide sequence encoding an enzyme that can condense one molecule of IPP with one molecule of DMAPP to form one molecule of geranyl pyrophosphate (“GPP”), e.g., a GPP synthase. Illustrative examples of nucleotide sequences encoding such an enzyme include, but are not limited to: (AF513111; Abies grandis), (AF513112; Abies grandis), (AF513113; Abies grandis), (AY534686; Antirrhinum majus), (AY534687; Antirrhinum majus), (Y17376; Arabidopsis thaliana), (AE016877, Locus AP11092; Bacillus cereus; ATCC 14579), (AJ243739; Citrus sinensis), (AY534745; Clarkia breweri), (AY953508; Ips pini), (DQ286930; Lycopersicon esculentum), (AF182828; Mentha x piperita), (AF182827; Mentha x piperita), (MP1249453; Mentha x piperita), (PZE431697, Locus CAD24425; Paracoccus zeaxanthinifaciens), (AY866498; Picrorhiza kurrooa), (AY351862; Vitis vinifera), and (AF203881, Locus AAF12843; Zymomonas mobilis).

In some embodiments, the host cell comprises a heterologous nucleotide sequence encoding an enzyme that can condense two molecules of IPP with one molecule of DMAPP, or add a molecule of IPP to a molecule of GPP, to form a molecule of famesyl pyrophosphate (“FPP”), e.g., a FPP synthase. Illustrative examples of nucleotide sequences that encode such an enzyme include, but are not limited to: (ATU80605; Arabidopsis thaliana), (ATHFPS2R; Arabidopsis thaliana), (AAU36376; Artemisia annua), (AF461050; Bos taurus), (D00694; Escherichia coli K-12), (AE009951, Locus AAL95523; Fusobacterium nucleatum subsp. nucleatum ATCC 25586), (GFFPPSGEN; Gibberella fujikuroi), (CP000009, Locus AAW60034; Gluconobacter oxydans 621H), (AF019892; Helianthus annuus), (HUMFAPS; Homo sapiens), (KLPFPSQCR; Kluyveromyces lactis), (LAU15777; Lupinus albus), (LAU20771; Lupinus albus), (AF309508; Mus musculus), (NCFPPSGEN; Neurospora crassa), (PAFPS1; Parthenium argentatum), (PAFPS2; Parthenium argentatum), (RATFAPS; Rattus norvegicus), (YSCFPP; Saccharomyces cerevisiae), (D89104; Schizosaccharomyces pombe), (CP000003, Locus AAT87386; Streptococcus pyogenes), (CP000017, Locus AAZ51849; Streptococcus pyogenes), (NC_008022, Locus YP_598856; Streptococcus pyogenes MGAS10270), (NC 008023, Locus YP_600845; Streptococcus pyogenes MGAS2096), (NC 008024, Locus YP_602832; Streptococcus pyogenes MGAS10750), (MZEFPS; Zea mays), (AE000657, Locus AAC06913; Aquifex aeolicus VF5), (NM_202836; Arabidopsis thaliana), (D84432, Locus BAA12575; Bacillus subtilis), (U12678, Locus AAC28894; Bradyrhizobium japonicum USDA 110), (BACFDPS; Geobacillus stearothermophilus), (NC 002940, Locus NP_873754; Haemophilus ducreyi 35000HP), (L42023, Locus AAC23087; Haemophilus influenzae Rd KW20), (J05262; Homo sapiens), (YP_395294; Lactobacillus sakei subsp. sakei 23K), (NC_005823, Locus YP_000273; Leptospira interrogans serovar Copenhageni str. Fiocruz L1-130), (AB003187; Micrococcus luteus), (NC_002946, Locus YP_208768; Neisseria gonorrhoeae FA 1090), (U00090, Locus AAB91752; Rhizobium sp. NGR234), (J05091; Saccharomyces cerevisae), (CP000031, Locus AAV93568; Silicibacter pomeroyi DSS-3), (AE008481, Locus AAK99890; Streptococcus pneumoniae R6), and (NC_004556, Locus NP 779706; Xylella fastidiosa Temeculal).

In some embodiments, the host cell further comprises a heterologous nucleotide sequence encoding an enzyme that can combine IPP and DMAPP or IPP and FPP to form geranylgeranyl pyrophosphate (“GGPP”). Illustrative examples of nucleotide sequences that encode such an enzyme include, but are not limited to: (ATHGERPYRS; Arabidopsis thaliana), (BT005328; Arabidopsis thaliana), (NM_119845; Arabidopsis thaliana), (NZ_AAJM01000380, Locus ZP_00743052; Bacillus thuringiensis serovar israelensis, ATCC 35646 sq1563), (CRGGPPS; Catharanthus roseus), (NZ_AABF02000074, Locus ZP_00144509; Fusobacterium nucleatum subsp. vincentii, ATCC 49256), (GFGGPPSGN; Gibberella fujikuroi), (AY371321; Ginkgo biloba), (AB055496; Hevea brasiliensis), (AB017971; Homo sapiens), (MC1276129; Mucor circinelloides f lusitanicus), (AB016044; Mus musculus), (AABX01000298, Locus NCU01427; Neurospora crassa), (NCU20940; Neurospora crassa), (NZ_AAKL01000008, Locus ZP_00943566; Ralstonia solanacearum UW551), (AB118238; Rattus norvegicus), (SCU31632; Saccharomyces cerevisiae), (AB016095; Synechococcus elongates), (SAGGPS; Sinapis alba), (SSOGDS; Sulfolobus acidocaldarius), (NC_007759, Locus YP_461832; Syntrophus aciditrophicus SB), (NC_006840, Locus YP_204095; Vibrio fischeri ES 114), (NM_112315; Arabidopsis thaliana), (ERWCRTE; Pantoea agglomerans), (D90087, Locus BAA14124; Pantoea ananatis), (X52291, Locus CAA36538; Rhodobacter capsulatus), (AF195122, Locus AAF24294; Rhodobacter sphaeroides), and (NC_004350, Locus NP_721015; Streptococcus mutans UA159).

While examples of the enzymes of the mevalonate pathway are described above, in certain embodiments, enzymes of the DXP pathway can be used as an alternative or additional pathway to produce DMAPP and IPP in the host cells, compositions and methods described herein. Enzymes and nucleic acids encoding the enzymes of the DXP pathway are well-known and characterized in the art, e.g., WO 2012/135591 A2.

### 6.6 Methods of Producing Steviol Glycosides

In another aspect, provided herein is a method for the production of a steviol glycoside, the method comprising the steps of (a) culturing a population of any of the genetically modified host cells described herein that are capable of producing a steviol glycoside in a medium with a carbon source under conditions suitable for making the steviol glycoside compound; and (b) recovering said steviol glycoside compound from the medium.

In some embodiments, the genetically modified host cell produces an increased amount of the steviol glycoside compared to a parent cell not comprising the one or more modifications, or a parent cell comprising only a subset of the one or more modifications of the genetically modified host cell, but is otherwise genetically identical. In some embodiments, the increased amount is at least 1%, 5%, 10%, 15%, 20%, 25%, 30%, 35%, 40%, 45%, 50%, 55%, 60%, 65%, 70%, 75%, 80%, 85%, 90%, 95%, 100% or greater than 100%, as measured, for example, in yield, production, and/or productivity, in grams per liter of cell culture, milligrams per gram of dry cell weight, on a per unit volume of cell culture basis, on a per unit dry cell weight basis, on a per unit volume of cell culture per unit time basis, or on a per unit dry cell weight per unit time basis.

In some embodiments, the host cell produces an elevated level of a steviol glycoside that is greater than about 1 grams per liter of fermentation medium. In some embodiments, the host cell produces an elevated level of a steviol glycoside that is greater than about 5 grams per liter of fermentation medium. In some embodiments, the host cell produces an elevated level of a steviol glycoside that is greater than about 10 grams per liter of fermentation medium. In some embodiments, the steviol glycoside is produced in an amount from about 10 to about 50 grams, from about 10 to about 15 grams, more than about 15 grams, more than about 20 grams, more than about 25 grams, or more than about 30 grams per liter of cell culture.

In some embodiments, the host cell produces an elevated level of a steviol glycoside that is greater than about 50 milligrams per gram of dry cell weight. In some such embodiments, the steviol glycoside is produced in an amount from about 50 to about 1500 milligrams, more than about 100 milligrams, more than about 150 milligrams, more than about 200 milligrams, more than about 250 milligrams, more than about 500 milligrams, more than about 750 milligrams, or more than about 1000 milligrams per gram of dry cell weight.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by a parent cell, on a per unit volume of cell culture basis.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by the parent cell, on a per unit dry cell weight basis.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by the parent cell, on a per unit volume of cell culture per unit time basis.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by the parent cell, on a per unit dry cell weight per unit time basis.

In most embodiments, the production of the elevated level of steviol glycoside by the host cell is inducible by the presence of an inducing compound. Such a host cell can be manipulated with ease in the absence of the inducing compound. The inducing compound is then added to induce the production of the elevated level of steviol glycoside by the host cell. In other embodiments, production of the elevated level of steviol glycoside by the host cell is inducible by changing culture conditions, such as, for example, the growth temperature, media constituents, and the like.

### 6.7 Culture Media and Conditions

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing steviol glycosides provided herein may be performed in a suitable culture medium (e.g., with or without pantothenate supplementation) in a suitable container, including but not limited to a cell culture plate, a microtiter plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wiley-VCH Verlag GmbH & Co. KDaA, Weinheim, Germany.

In some embodiments, the culture medium is any culture medium in which a genetically modified microorganism capable of producing an steviol glycoside can subsist, i.e., maintain growth and viability. In some embodiments, the culture medium is an aqueous medium comprising assimilable carbon, nitrogen and phosphate sources. Such a medium can also include appropriate salts, minerals, metals and other nutrients. In some embodiments, the carbon source and each of the essential cell nutrients, are added incrementally or continuously to the fermentation media, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable media for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, xylose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose, in the culture medium is sufficient to promote cell growth, but is not so high as to repress growth of the microorganism used. Typically, cultures are run with a carbon source, such as glucose, being added at levels to achieve the desired level of growth and biomass. In other embodiments, the concentration of a carbon source, such as glucose, in the culture medium is greater than about 1 g/L, preferably greater than about 2 g/L, and more preferably greater than about 5 g/L. In addition, the concentration of a carbon source, such as glucose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals or growth promoters. Such other compounds can also be present in carbon, nitrogen or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the culture medium is greater than about 1.0 g/L, preferably greater than about 2.0 g/L and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 ml/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture media can include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or steviol glycoside production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of steviol glycoside. For example, prior to inoculation of the culture medium with an inoculum, the culture medium can be brought to and maintained at a temperature in the range of from about 20° C. to about 45° C., preferably to a temperature in the range of from about 25° C. to about 40° C., and more preferably in the range of from about 28° C. to about 32° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

In some embodiments, the carbon source concentration, such as the glucose concentration, of the culture medium is monitored during culture. Glucose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. The carbon source concentration is typically maintained below the level at which cell growth inhibition occurs. Although such concentration may vary from organism to organism, for glucose as a carbon source, cell growth inhibition occurs at glucose concentrations greater than at about 60 g/L, and can be determined readily by trial. Accordingly, when glucose is used as a carbon source the glucose is preferably fed to the fermentor and maintained below detection limits. Alternatively, the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

Other suitable fermentation medium and methods are described in, e.g., WO 2016/196321.

### 6.8 Fermentation Compositions

In another aspect, provided herein are fermentation compositions comprising a genetically modified host cell described herein and steviol glycosides produced from genetically modified host cell. The fermentation compositions may further comprise a medium. In certain embodiments, the fermentation compositions comprise a genetically modified host cell, and further comprise Reb A, Reb D, and Reb M. In certain embodiments, the fermentation compositions provided herein comprise Reb M as a major component of the steviol glycosides produced from the genetically modified host cell. In certain embodiments, the fermentation compositions comprise Reb A, Reb D, and Reb M at a ratio of at least 1:7:50. In certain embodiments, the fermentation compositions comprise Reb A, Reb D, and Reb M at a ratio of at least 1:7:50 to 1:100:1000. In certain embodiments, the fermentation compositions comprise a ratio of at least 1:7:50 to 1:200:2000. In certain embodiments, the ratio of Reb A, Reb D, and Reb M are based on the total content of steviol glycosides that are associated with the genetically modified host cell and the medium. In certain embodiments, the ratio of Reb A, Reb D, and Reb M are based on the total content of steviol glycosides in the medium. In certain embodiments, the ratio of Reb A, Reb D, and Reb M are based on the total content of steviol glycosides that are associated with the genetically modified host cell.

In certain embodiments, the fermentation compositions provided herein contain Reb M2 at an undetectable level. In certain embodiments, the fermentation compositions provided herein contain non-naturally occurring steviol glycosides at an undetectable level.

### 6.9 Recovery of Steviol Glycosides

Once the steviol glycoside is produced by the host cell, it may be recovered or isolated for subsequent use using any suitable separation and purification methods known in the art. In some embodiments, a clarified aqueous phase comprising the steviol glycoside is separated from the fermentation by centrifugation. In other embodiments, a clarified aqueous phase comprising the steviol glycoside is separated from the fermentation by adding a demulsifier into the fermentation reaction. Illustrative examples of demulsifiers include flocculants and coagulants.

The steviol glycoside produced in these cells may be present in the culture supernatant and/or associated with the host cells. In embodiments where some of the steviol glycoside is associated with the host cell, the recovery of the steviol glycoside may comprise a method of improving the release of the steviol glycosides from the cells. In some embodiments, this could take the form of washing the cells with hot water or buffer treatment, with or without a surfactant, and with or without added buffers or salts. In some embodiments, the temperature is any temperature deemed suitable for releasing the steviol glycosides. In some embodiments, the temperature is in a range from 40 to 95° C.; or from 60 to 90° C.; or from 75 to 85° C. In some embodiments, the temperature is 40, 45, 50, 55, 65, 70, 75, 80, 85, 90, or 95° C. In some embodiments physical or chemical cell disruption is used to enhance the release of steviol glycosides from the host cell. Alternatively and/or subsequently, the steviol glycoside in the culture medium can be recovered using an isolation unit operations including, but not limited to solvent extraction, membrane clarification, membrane concentration, adsorption, chromatography, evaporation, chemical derivatization, crystallization, and drying.

### 6.10 Methods of Making Genetically Modified Cells

Also provided herein are methods for producing a host cell that is genetically engineered to comprise one or more of the modifications described above, e.g., one or more nucleic heterologous nucleic acids encoding Stevia rebaudiana kaurenoic acid hydroxylase, and/or biosynthetic pathway enzymes, e.g., for a steviol glycoside compound. Expression of a heterologous enzyme in a host cell can be accomplished by introducing into the host cells a nucleic acid comprising a nucleotide sequence encoding the enzyme under the control of regulatory elements that permit expression in the host cell. In some embodiments, the nucleic acid is an extrachromosomal plasmid. In other embodiments, the nucleic acid is a chromosomal integration vector that can integrate the nucleotide sequence into the chromosome of the host cell. In other embodiments, the nucleic acid is a linear piece of double stranded DNA that can integrate via homology the nucleotide sequence into the chromosome of the host cell.

Nucleic acids encoding these proteins can be introduced into the host cell by any method known to one of skill in the art without limitation (see, for example, Hinnen et al. (1978) Proc. Natl. Acad. Sci. USA 75:1292-3; Cregg et al. (1985) Mol. Cell. Biol. 5:3376-3385; Goeddel et al. eds, 1990, Methods in Enzymology, vol. 185, Academic Press, Inc; CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY). Exemplary techniques include, but are not limited to, spheroplasting, electroporation, PEG 1000 mediated transformation, and lithium acetate or lithium chloride mediated transformation.

The amount of an enzyme in a host cell may be altered by modifying the transcription of the gene that encodes the enzyme. This can be achieved for example by modifying the copy number of the nucleotide sequence encoding the enzyme (e.g., by using a higher or lower copy number expression vector comprising the nucleotide sequence, or by introducing additional copies of the nucleotide sequence into the genome of the host cell or by deleting or disrupting the nucleotide sequence in the genome of the host cell), by changing the order of coding sequences on a polycistronic mRNA of an operon or breaking up an operon into individual genes each with its own control elements, or by increasing the strength of the promoter or operator to which the nucleotide sequence is operably linked. Alternatively or in addition, the copy number of an enzyme in a host cell may be altered by modifying the level of translation of an mRNA that encodes the enzyme. This can be achieved for example by modifying the stability of the mRNA, modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of enzyme, altering expression of rare codon tRNAs used in the biosynthesis of the enzyme, and/or increasing the stability of the enzyme, as, for example, via mutation of its coding sequence.

The activity of an enzyme in a host cell can be altered in a number of ways, including, but not limited to, expressing a modified form of the enzyme that exhibits increased or decreased solubility in the host cell, expressing an altered form of the enzyme that lacks a domain through which the activity of the enzyme is inhibited, expressing a modified form of the enzyme that has a higher or lower Kcat or a lower or higher Km for the substrate, or expressing an altered form of the enzyme that is more or less affected by feed-back or feed-forward regulation by another molecule in the pathway.

In some embodiments, a nucleic acid used to genetically modify a host cell comprises one or more selectable markers useful for the selection of transformed host cells and for placing selective pressure on the host cell to maintain the foreign DNA.

In some embodiments, the selectable marker is an antibiotic resistance marker. Illustrative examples of antibiotic resistance markers include, but are not limited to, the BLA, NAT1, PAT, AUR1-C, PDR4, SMR1, CAT, mouse dhfr, HPH, DSDA, KAN^{R}, and SHBLE gene products. The BLA gene product from E. coli confers resistance to beta-lactam antibiotics (e.g., narrow-spectrum cephalosporins, cephamycins, and carbapenems (ertapenem), cefamandole, and cefoperazone) and to all the anti-gram-negative-bacterium penicillins except temocillin; the NAT1 gene product from S. noursei confers resistance to nourseothricin; the PAT gene product from S. viridochromogenes Tu94 confers resistance to bialophos; the A URI-C gene product from Saccharomyces cerevisiae confers resistance to Auerobasidin A (AbA); the PDR4 gene product confers resistance to cerulenin; the SMR1 gene product confers resistance to sulfometuron methyl; the CAT gene product from Tn9 transposon confers resistance to chloramphenicol; the mouse dhfr gene product confers resistance to methotrexate; the HPH gene product of Klebsiella pneumonia confers resistance to Hygromycin B; the DSDA gene product of E. coli allows cells to grow on plates with D-serine as the sole nitrogen source; the KAN^{R} gene of the Tn903 transposon confers resistance to G418; and the SHBLE gene product from Streptoalloteichus hindustanus confers resistance to Zeocin (bleomycin). In some embodiments, the antibiotic resistance marker is deleted after the genetically modified host cell disclosed herein is isolated.

In some embodiments, the selectable marker rescues an auxotrophy (e.g., a nutritional auxotrophy) in the genetically modified microorganism. In such embodiments, a parent microorganism comprises a functional disruption in one or more gene products that function in an amino acid or nucleotide biosynthetic pathway and that when non-functional renders a parent cell incapable of growing in media without supplementation with one or more nutrients. Such gene products include, but are not limited to, the HIS3, LEU2, LYS1, LYS2, MET15, TRP1, ADE2, and URA3 gene products in yeast. The auxotrophic phenotype can then be rescued by transforming the parent cell with an expression vector or chromosomal integration construct encoding a functional copy of the disrupted gene product, and the genetically modified host cell generated can be selected for based on the loss of the auxotrophic phenotype of the parent cell. Utilization of the URA3, TRP1, and LYS2 genes as selectable markers has a marked advantage because both positive and negative selections are possible. Positive selection is carried out by auxotrophic complementation of the URA3, TRP1, and LYS2 mutations, whereas negative selection is based on specific inhibitors, i.e., 5-fluoro-orotic acid (FOA), 5-fluoroanthranilic acid, and aminoadipic acid (aAA), respectively, that prevent growth of the prototrophic strains but allows growth of the URA3, TRP1, and LYS2 mutants, respectively. In other embodiments, the selectable marker rescues other non-lethal deficiencies or phenotypes that can be identified by a known selection method.

Described herein are specific genes and proteins useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.” Codon optimization for other host cells can be readily determined using codon usage tables or can be performed using commercially available software, such as CodonOp (www.idtdna.com/CodonOptfrom) from Integrated DNA Technologies.

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8).

Those of skill in the art will recognize that, due to the degenerate nature of the genetic code, a variety of DNA molecules differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA molecules of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for the compositions and methods provided herein are encompassed by the disclosure. In some embodiments, two proteins (or a region of the proteins) are substantially homologous when the amino acid sequences have at least about 30%, 40%, 50%, 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol Biol 25: 365-89).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used comparing a molecule sequence to a database containing a large number of sequences from different organisms is the computer program BLAST. When searching a database containing sequences from a large number of different organisms, it is typical to compare amino acid sequences.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast.

In addition, genes encoding these enzymes can be identified from other fungal and bacterial species and can be expressed for the modulation of this pathway. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia. coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous UDP glycosyltransferases, KAH, or any biosynthetic pathway genes, proteins, or enzymes, techniques may include, but are not limited to, cloning a gene by PCR using primers based on a published sequence of a gene/enzyme of interest, or by degenerate PCR using degenerate primers designed to amplify a conserved region among a gene of interest. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above-mentioned databases in accordance with the teachings herein.

### 7. EXAMPLES

### Example 1. Yeast Transformation Methods

Each DNA construct was integrated into Saccharomyces cerevisiae (CEN.PK2) using standard molecular biology techniques for an optimized lithium acetate transformation. Briefly, cells were grown overnight in yeast extract peptone dextrose (YPD) media at 30° C. with shaking (200 rpm), diluted to an OD600 of 0.1 in 100 mL YPD, and grown to an OD600 of 0.6-0.8. For each transformation, 5 mL of culture was harvested by centrifugation, washed in 5 mL of sterile water, spun down again, resuspended in 1 mL of 100 mM lithium acetate, and transferred to a microcentrifuge tube. Cells were spun down (13,000×g) for 30 seconds, the supernatant was removed, and the cells were resuspended in a transformation mix consisting of 240 μL 50% PEG, 36 μL 1 M lithium acetate, 10 μL boiled salmon sperm DNA, and 74 μL of donor DNA. The donor DNA included a plasmid carrying the F-CphI endonuclease gene expressed under the yeast TDH3 promoter for expression (see Example 4). Following a heat shock at 42° C. for 40 minutes, cells were recovered overnight in YPD media containing the appropriate antibiotic to select for cells that have taken up the F-CphI plasmid. After recovery over night, the cells are briefly spun down by centrifugation and plated on YPD media containing the appropriate antibiotic to select for cells that have taken up the F-CphI plasmid. DNA integration was confirmed by colony PCR with primers specific to the integrations.

### Example 2: Generation of a Base Yeast Strain Capable of High Flux to Farnesylpyrophosphate (FPP) and the Isoprenoid Farnesene

A farnesene production strain was created from a wild-type Saccharomyces cerevisiae strain (CEN.PK2) by expressing the genes of the mevalonate pathway under the control of GAL1 or GAL10 promoters. This strain comprised the following chromosomally integrated mevalonate pathway genes from S. cerevisiae: acetyl-CoA thiolase, HMG-CoA synthase, HMG-CoA reductase, mevalonate kinase, phosphomevalonate kinase, mevalonate pyrophosphate decarboxylase, and IPP:DMAPP isomerase. In addition, the strain contained multiple copies of famesene synthase from Artemisia annua, also under the control of either GAL1 or GAL10 promoters. All heterologous genes described herein were codon optimized using publicly available or other suitable algorithms. The strain also contained a deletion of the GAL80 gene, and the ERG9 gene encoding squalene synthase was downregulated by replacing the native promoter with promoter of the yeast gene MET3 (Westfall et al., Proc. Natl. Acad. Sci. USA 109(3), 2012, pp. E111-E118). Examples of how to create S. cerevisiae strains with high flux to isoprenoids are described in the U.S. Pat. Nos. 8,415,136 and 8,236,512 which are incorporated herein in their entireties.

### Example 3. Generation of a Base Yeast Strain Capable of High Flux to Reb M

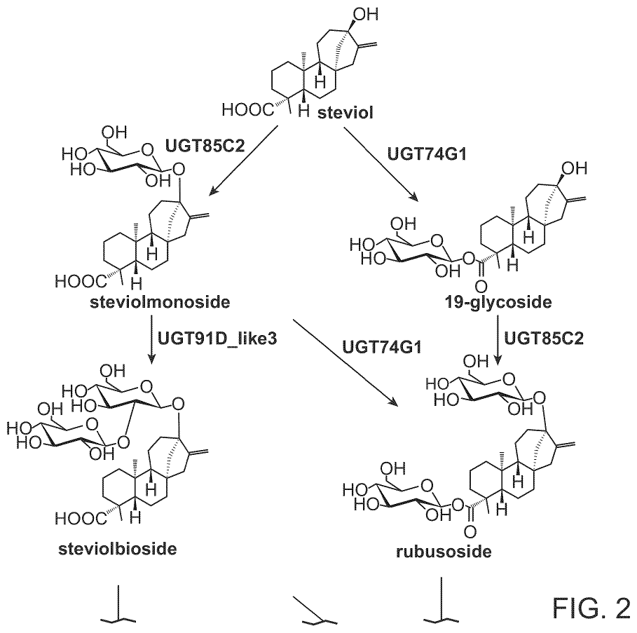

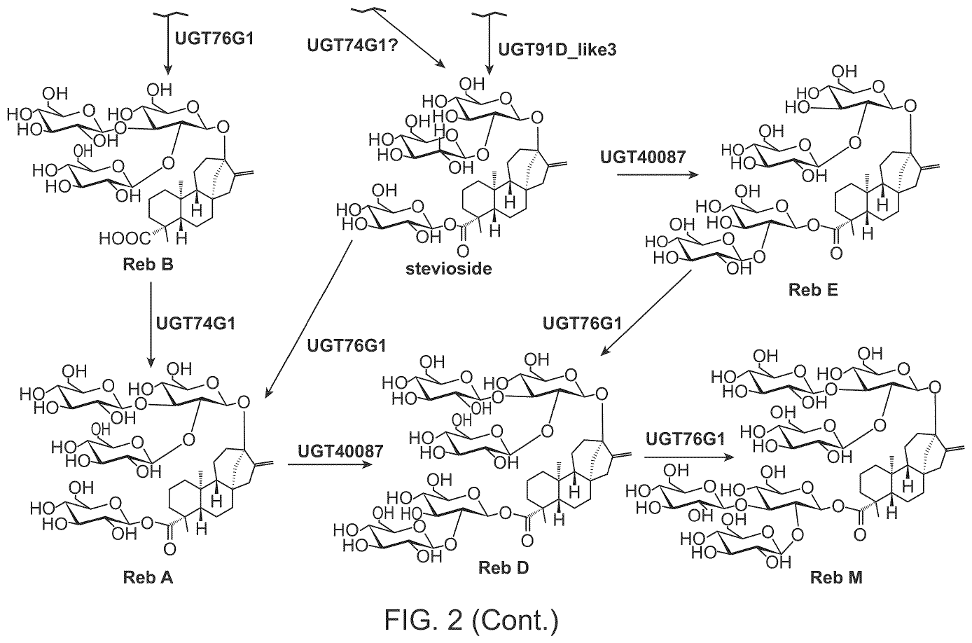

FIG. 1 shows an exemplary biosynthetic pathway from FPP to steviol. FIG. 2 shows an exemplary biosynthetic pathway from steviol to the glycoside Reb M. To convert the famesene base strain described above to have high flux to the C20 isoprenoid kaurene, four copies of a geranylgeranylpyrophosphate synthase (GGPPS) were integrated into the genome, followed by two copies of a copalyldiphosphate synthase and a single copy of a kaurene synthase. At this point all copies of famesene synthase were removed from the strain. Once the new strain was confirmed to make ent-kaurene, the remaining genes for converting ent-kaurene to Reb M were inserted into the genome. Table 1 lists all genes and promoters used to convert FPP to Reb M. Each gene after kaurene synthase was integrated as a single copy, except for the Sr.KAH enzyme for which two gene copies were integrated. The strain containing all genes described in Table 1 primarily produced Reb M.

TABLE 1
Genes, promoters, and amino acid sequences of the enzymes used
to convert FPP to Reb M.

| Enzyme name | SEQ ID | Promoter |
|---|---|---|
| Bt.GGPPS | SEQ ID NO: 9 | PGAL1 |
| ent-Os, CDPS | SEQ ID NO: 10¹ | PGAL1 |
| ent-Pg.Ks | SEQ ID NO: 11 | PGAL1 |
| Ps.KO | SEQ ID NO: 12 | PGAL1 |
| Sr.KAH | SEQ ID NO: 13 | PGAL1 |
| At.CPR | SEQ ID NO: 14 | PGAL3 |
| UGT85C2 | SEQ ID NO: 15 | PGAL10 |
| UGT74G1 | SEQ ID NO: 16 | PGAL1 |
| UGT91D_like3 | SEQ ID NO: 17 | PGAL1 |
| UGT76G1 | SEQ ID NO: 18 | PGAL10 |
| UGT40087 | SEQ ID NO: 19 | PGAL1 |

¹First 65 amino acids removed and replaced with methionine

### Example 4. Generation of a Strain to Screen for Steviol Glycoside Transporters

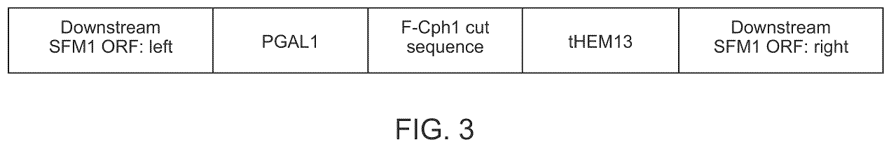

To rapidly screen for steviol glycoside transporters in vivo in a strain producing Reb M, a landing pad was inserted into the strain described above. The landing pad consisted of 500 bp of locus-targeting DNA sequences on either end of the construct to the genomic region downstream of the SFM1 open reading frame (see FIG. 3). Internally, the landing pad contained a GAL1 promoter and a yeast terminator flanking an endonuclease recognition site (F-CphI).

### Example 5: Yeast Culturing Conditions

Yeast colonies with an overexpressed transporter protein were picked into 96-well microtiter plates containing Bird Seed Media (BSM, originally described by van Hoek et al., Biotechnology and Bioengineering 68(5), 2000, pp. 517-523) with 20 g/L sucrose, 3.75 g/L ammonium sulfate, and 1 g/L lysine. Cells were cultured at 28° C. in a high capacity microtiter plate incubator shaking at 1000 rpm and 80% humidity for 3 days until the cultures reached carbon exhaustion. The growth-saturated cultures were subcultured into fresh plates containing BSM with 40 g/L sucrose and 3.75 g/L ammonium sulfate by taking 14.4 μL from the saturated cultures and diluting into 360 μL of fresh media. Cells in the production media were cultured at 30° C. in a high capacity microtiter plate shaker at 1000 rpm and 80% humidity for an additional 3 days prior to extraction and analysis.

### Example 6: Whole Cell Broth Sample Prep Conditions for Analysis of Steviol Glycosides

To analyze the amount of all steviol glycosides produced in the culture, upon culturing completion the whole cell broth was diluted with 628 μL of 100% ethanol, sealed with a foil seal, and shaken at 1250 rpm for 30 seconds to extract the steviol glycosides. 314 μL of water was added to each well directly to dilute the extraction. The plate was briefly centrifuged to pellet solids. An internal standard, 208 μL of 50:50 ethanol:water mixture containing 0.48 mg/L rebaudioside N, was transferred to a new 250 μL assay plate and 2 μL of the culture/ethanol mixture was added to the assay plate. A foil seal was applied to the plate prior to analysis.

### Example 7: Culture Supernatant Sample Prep Conditions for Analysis of Steviol Glycosides

To analyze the amount of all steviol glycosides produced and excreted into the culture media, upon culturing completion the whole-cell broth was centrifuged for 5 minutes at 2000×g to pellet the cells. A 240 μL aliquot of the resulting supernatant was transferred to an empty 96-well microtiter plate. The supernatant samples were diluted with 480 μL of 100% ethanol, sealed with a foil seal, and shaken at 1250 rpm for 30 seconds to extract the steviol glycosides. To dilute the extraction 240 μL of water was added to each well. The plate was briefly centrifuged to pellet any solids. An internal standard, 208 μL of 50:50 ethanol:water mixture containing 0.48 mg/L rebaudioside N, was transferred to a new 250 μL assay plate and 2 μL of the culture/ethanol mixture was added to the assay plate. A foil seal was applied to the plate prior to analysis.

### Example 8: Analytical Methods

Samples for steviol glycoside measurements were analyzed by mass spectrometer (Agilent 6470-QQQ) with a RapidFire 365 system autosampler with C8 cartridge using the configurations shown in Tables 2 and 3.

TABLE 2
RapidFire 365 system configuration

| | |
|---|---|
| Pump 1, Line A: 2 mM | 100% A, 1.5 mL/min |
| ammonium formate in water | |
| Pump 2, Line A: 35% | 100% A, 1.5 mL/min |
| acetonitrile in water | |
| Pump 3, Line A: 80% | 100% A, 0.8 mL/min |
| acetonitrile in water | |
| State 1: Aspirate | 600 ms |
| State 2: Load/Wash | 3000 ms |
| State 3: Extra wash | 1500 ms |
| State 4: Elute | 5000 ms |
| State 5: Re-equilibrate | 1000 ms |

TABLE 3
6470-QQQ MS method configurations

| Ion Source | AJS ESI | |
|---|---|---|
| Time Filtering peak width | 0.02 | min |
| Stop Time | No limit/as pump | |
| Scan Type | MRM | |
| Diverter Valve | To MS | |
| Delta EMV | (+)0/(−)300 | |
| Ion Mode (polarity) | Negative | |
| Gas Temp | 250° C. | |
| Gas Flow | 11 | L/min |
| Nebulizer | 30 | psi |
| Sheath Gas Temp | 350° C. | |
| Sheath Gas Flow | 11 | L/min |
| Negative Capillary V | 2500 | V |

The peak areas from a chromatogram from a mass spectrometer were used to generate the calibration curve. The molar ratios of relevant compounds were determined by quantifying the amount in moles of each compound through external calibration using an authentic standard, and then taking the appropriate ratios.

### Example 9. Screening for Transporters Capable of Increasing Titers of Steviol Glycosides In Vivo

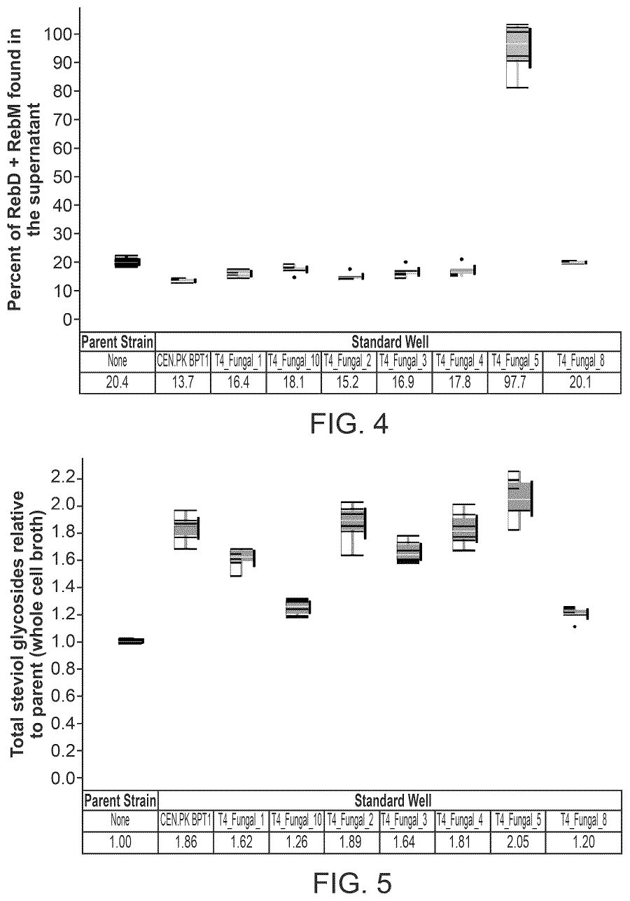

In the Reb M-producing strain without additional transporters expressed, approximately 80% of the higher molecular weight steviol glycosides Reb D and Reb M were found to be associated with the biomass (see FIG. 4). This biomass association is likely attributed to Reb D and Reb M not being efficiently transported out of the cell and retained in the cytoplasm. The accumulation of Reb D and Reb M could result in product inhibition which would decrease the carbon flux through the steviol glycoside metabolic pathway. Therefore, expression of one or more transporters that will transport steviol glycosides (especially Reb D and Reb M) out of the cytoplasm and into the media (supernatant) is expected to relieve product inhibition and thereby increase carbon flux through the pathway, resulting in higher steviol glycoside titers. To identify transporters capable of exporting higher molecular weight steviol glycosides out of the cell and thus relieving product inhibition, we screened a number of transporters identified from a variety of fungi for the ability to increase total steviol glycoside titers, particularly the titers of higher molecular weight glycosides (i.e. Reb D and Reb M).

All proteins annotated to be a transporter from the S. cerevisiae genome were amplified via PCR, using CEN.PK2 as the genomic DNA source. Each PCR primer had 40 bp of flanking homology to the PGAL1 and yeast terminator DNA sequences in the landing pad (see FIG. 3) added to the ends to facilitate homologous recombination of the amplified gene into the landing pad. In addition to screening all the endogenous S. cerevisiae transport proteins found in CEN.PK2, an extended bioinformatics search was performed for ABC-transporter proteins from a small number of fungi and additional S. cerevisiae strains.

To make a library of fungal ABC-transporters, we first obtained amino acid sequences from the publication “Phylogenetic Analysis of Fungal ABC Transporters” by Kovalchuk and Driessen (Kovalchuk and Driessen, BMC Genomics, 11, 2010, pp. 177-197) in which a phylogenetic analysis of ABC transporters was performed for 27 fungal species. From this literature source, a total of 610 amino acid sequences were chosen, which included all transporters designated as belonging to the ABC-C, ABC-D, and ABC-G subfamilies. Next, we developed in-house BLAST databases for the following fungi: (1) Hansenula polymorpha DL-1 (NRRL-Y-7560), (2) Yarrowia lipolytica ATCC 18945, (3) Arxula adeninivorans ATCC 76597, (4) S. cerevisiae CAT-1, (5) Lipomyces starkeyi ATCC 58690, (6) Kluyveromyces marxianus, (7) Kluyveromyces marxianus DMKU3-1042, (8) Komagataella phaffii NRRL Y-11430, (9) S. cerevisiae MBG3370, (10) S. cerevisiae MBG3373, (11) Kluyveromyces lactis ATCC 8585, (12) Candida utilis ATCC 22023, (13) Pichia pastoris ATCC 28485, and (14) Aspergillus oryzae NRRL5590.

For organisms in which we already had in-house nucleotide ORF sequences from a de novo genomic sequencing, assembling, and annotation effort, we applied tBLASTn using Biopython. The tBLASTn algorithm allowed for rapid alignments of protein sequences (in this case the 610 seed sequences from Kovalchuk and Driessen (BMC Genomics, 11, 2010, pp. 177-197)) with translated DNA of the nucleotide ORF sequences for each organism in all six possible reading frames using BLAST. tBLASTn parameters were standard with evalue=1 e⁻²⁵ (see Table 4). All computations were executed via the biopython API (v 1.70 downloaded from PyPI) using Python 2.7.12 and Ubuntu 16.04.5 LTS (GNU/Linux 4.4.0-138-generic x86_64). Hits were subsequently filtered to ensure a global alignment of at least 2000 nucleotides. All matches meeting these criteria were taken to the next step of the workflow.

TABLE 4
tBLASTn default parameters

| tBLASTn (2.2.31 BLAST + | |
|---|---|
| release) option | Setting used |
| word_size | 3 |
| gapopen | 11 |
| gapextend | 1 |
| matrix | BLOSUM62 |
| threshold | 13 |
| seg | 12 2.2 2.5 |
| soft_masking | FALSE |
| lcase_masking | N/A |
| db_soft_mask | None |
| db_hard_mask | None |
| xdrop_gap_final | 25 |
| window_size | 40 |
| db_gen_code | 1 |
| max_intron_length | 0 |
| comp_based_stats | 2 |

For the remainder of the organisms for which there was not in-house genomic sequence, the entire proteome of the organism was obtained from Uniprot using the Uniprot API in order to create a database for a BLASTp search. In most cases Uniprot had an exact entry for a species for which we had in-house genomic DNA, but in other cases there was a close but not exact match to the fungal strains in-house. In the latter cases we relied on the high probability that the gene sequences would be similar enough that primers designed against the Uniprot reference would still amplify the in-house genomic DNA. We then applied BLASTp using Biopython to the Uniprot derived database. BLAST parameters were standard, with evalue=0.001 (see Table 5). A subsequent filtering was performed based on a percent identity cutoff of ≥40%, and a percent aligned length cutoff of ≥60%. All computations were executed via the biopython API (v 1.70 downloaded from PyPI) using Python 2.7.12 and Ubuntu 16.04.5 LTS (GNU/Linux 4.4.0-138-generic x86_64). Hits had to match at least one of the 610 seed sequences from the reference. Hits were then converted to nucleotide sequence using the Uniprot ID mapping service to EMBL identifiers. The European Molecular Biology Laboratory allows for extraction of nucleotide sequences from a Uniprot entry. We took any hits fitting these criteria to the next step of the workflow.

TABLE 5
BLASTp default parameters

| BLASTp (2.2.31 BLAST + | |
|---|---|
| release) option | Setting used |
| word_size | 3 |
| word_size | 2 |
| word size | 6 |
| gapopen | 11 |
| gapextend | 1 |
| gapopen | 9 |
| gapextend | 1 |
| matrix | BLOSUM62 |
| matrix | PAM30 |
| threshold | 11 |
| threshold | 16 |
| Threshold | 21 |
| comp_based_stats | 2 |
| comp_based_stats | 0 |
| seg | No |
| soft_masking | FALSE |
| lcase_masking | N/A |
| db_soft_mask | None |
| db_hard_mask | None |
| xdrop_gap_final | 25 |
| window_size | 40 |
| window_size | 15 |
| use_sw_tback | N/A |

Once all nucleotide sequences had been identified, primers were designed to amplify each complete ORF via PCR. Each PCR primer had 40 bp of flanking homology to the PGAL1 and yeast terminator DNA sequences in the landing pad (FIG. 3) added to the ends to facilitate homologous recombination of the amplified gene into the landing pad. Each transporter gene was transformed individually as a single copy into the Reb M-producing yeast strain described above and screened for the ability to increase product titers when overexpressed in vivo.

### Example 11: Overexpression of Transporters that Lead to an Increase in Steviol Glycoside Production In Vivo

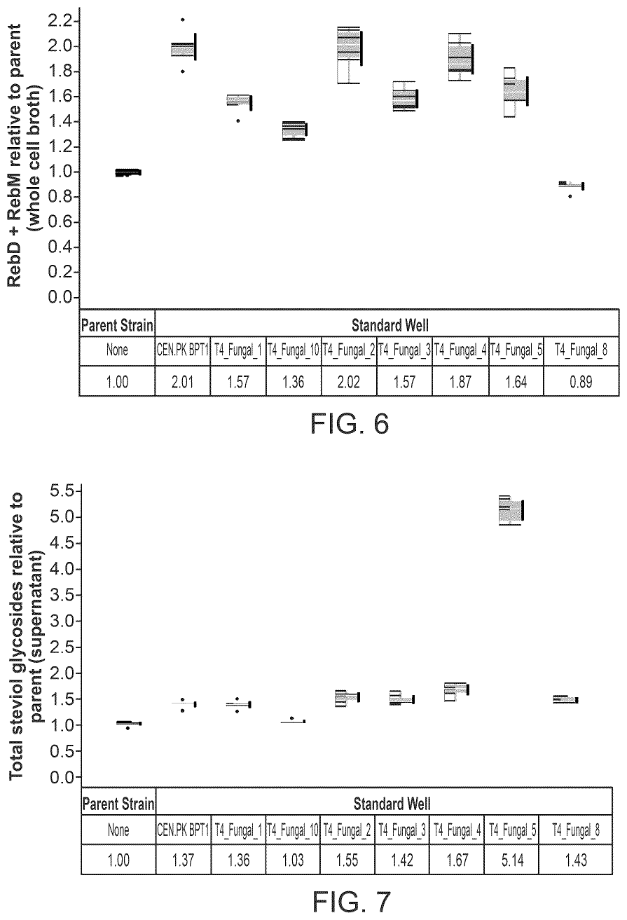

The in vivo S. cerevisiae transporter screen found eight transporters that statistically increased total steviol glycoside (TSG) production when overexpressed, compared to the parent Reb M strain that contained no overexpressed transporter (see FIG. 5). TSG was calculated as the sum in micromoles of all steviol glycosides produced by the cell (as measured by whole cell broth extraction). All of the identified transporters fall into the class of transporters known as ABC-transporters. Overexpression of these transporters increased TSG from 20% to two-fold over parent. Increases in TSG by transporter overexpression could be due to increased transport of all steviol glycosides, or just a subset of steviol glycosides. Therefore, the data was also analyzed to determine the effect transporter overexpression had on just the higher molecular weight steviol glycosides Reb D and Reb M. Of the eight transporters that increased TSG, seven of them also increased overall production of Reb D and Reb M, as shown in FIG. 6. Increases of Reb D and Reb M with overexpression of transporters ranged from 30% increase to two-fold increase.

### Example 12: Extracellular and Intracellular Transport of Steviol Glycosides

Seven out of eight Reb M strains harboring overexpressed transporters that resulted in more total steviol glycosides in the whole cell broth also increased the total steviol glycoside content in the supernatant (FIG. 7). While four of the transporters increased the total steviol glycosides in the whole cell broth by nearly two-fold (FIG. 5), the typical increase of TSG in the supernatant was less and ranged from 35 to 70% (FIG. 7). However, transporter T4_Fungal_5 increased the TSG in the supernatant approximately five-fold (FIG. 7). The data shown in FIGS. 5 and 7 demonstrates that strains with certain overexpressed transporters are making more TSG, but the increase in TSG is not always reflected with a linear increase in TSG in the supernatant.

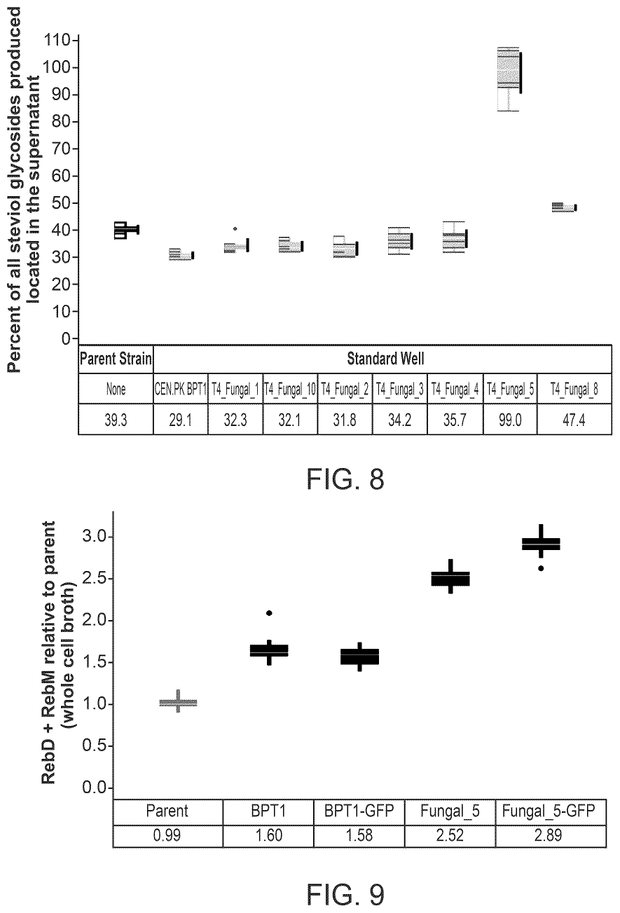

Looking explicitly at the fraction of total steviol glycosides produced that is located in the supernatant (FIG. 8) shows that the majority of the transporters (six out of eight) showed a lower proportion of TSG in the supernatant relative to parent. This suggests that the transporters were removing the steviol glycosides from the cytosol, thereby relieving product inhibition and allowing for greater product formation, but they were not transporting the steviol glycosides into the media. Instead, these transporters are most likely transporting the steviol glycosides into the vacuole or some other cellular compartment. In contrast, transporter T4_Fungal_5 resulted in nearly 100% of the TSG produced being located in the supernatant (FIG. 8). This indicates that T4_Fungal_5 is likely a plasma membrane transporter that is capable of removing steviol glycosides from the cell's cytoplasm and transporting it out of the cell and into the media. In addition, the data in FIG. 4 shows that transporter T4_Fungal_5 exports the higher molecular weight steviol glycosides Reb D and Reb M out of the cell and into the media; indeed, nearly 100% of both Reb D and Reb M were located in the supernatant fraction.

One of the hits from the transporter screen was the endogenous S. cerevisiae ABC-transporter BPT1. This protein is annotated in the Saccharomyces Genome Database to be localized to the vacuole. Transporters T4_Fungal_2 and T4_Fungal_4 have protein sequences that are 99% identical to CEN.PK2 BPT1 and are derived from S. cerevisiae strains CAT-1 and MBG3373, respectively; they are alleles of BPT1. All other transporters are 30-43% identical in protein sequence to BPT1 and represent novel ABC-transporters that can transport steviol glycosides across membranes (see Table 6). Of the remaining non-BPT1 transporters that export out steviol glycosides, no protein sequence is higher than 53% identical to any other protein, showing that the remaining five proteins are unique sequences.

TABLE 6
Percent identity of all transporters that increase steviol glycoside titers.

| | T4_Fungal_8 | T4_Fungal_1 | T4_Fungal_3 | T4_Fungal_5 | T4_Fungal_10 | CENPK_BPT1 | T4_Fungal_2 | T4_Fungal_4 |
|---|---|---|---|---|---|---|---|---|
| T4_Fungal_8 | 100 | 47.56 | 52.95 | 30.27 | 31.50 | 30.50 | 30.57 | 30.64 |
| T4_Fungal_1 | | 100 | 53.12 | 30.05 | 31.29 | 30.41 | 30.34 | 30.41 |
| T4_Fungal_3 | | | 100 | 31.53 | 33.43 | 32.36 | 32.43 | 32.50 |
| T4_Fungal_5 | | | | 100 | 31.74 | 31.05 | 30.89 | 30.89 |
| T4_Fungal_10 | | | | | 100 | 43.47 | 43.40 | 43.40 |
| CENPK_BPT1 | | | | | | 100 | 99.49 | 99.55 |
| T4_Fungal_2 | | | | | | | 100 | 99.81 |
| T4_Fungal_4 | | | | | | | | 100 |

### Example 13: BPT1 and T4_Fungal_5 Cellular Localization

To determine the cellular localization of overexpressed BPT1 and T4_Fungal_5 protein in the Reb M-producing strains, we created GFP-transporter fusion proteins. Each transporter (BPT1 or T4_Fungal_5) protein had a GFP protein fused to the C-terminal of the transporter; the GFP-transporter fusion proteins were expressed via a GAL1 promoter and contained a yeast terminator. Strains were constructed as outlined in Example 4, with the only difference being that a transporter-GFP fusion protein was used in place of the transporter-only protein. Cells with properly integrated transporter-GFP constructs were confirmed via colony PCR, cultured as in Example 5, and confirmed to have activity equivalent to the strains containing transporter without a C-terminal GFP tag (FIG. 9).

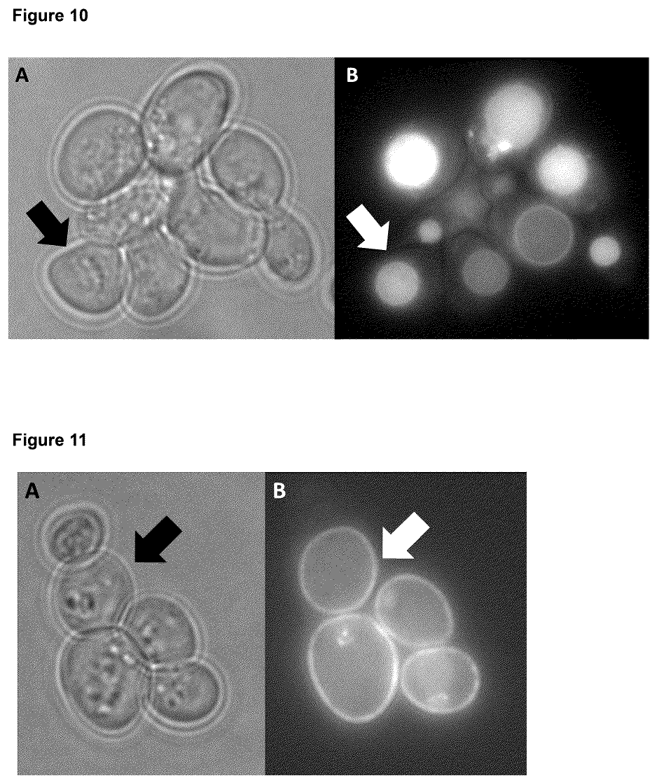

To visualize protein localization via GFP, cells were propagated as in Example 5 but were harvested after 2 days in production media for observation. Cells were washed twice with equal volumes of PBS and then resuspended to an OD600 of 1.0 in PBS. Cells were fixed using 1% agarose pads mounted on a glass slide and visualized at 100× magnification with an oil immersion using a standard fluorescence microscope at a 488 nm excitation or under bright field. Cells expressing BPT1 C-terminally tagged with GFP showed fluorescence patterns consistent with the fusion protein being localized to the vacuole (FIG. 10). This was the expected result, since it has been reported that BPT1 is normally localized to the vacuole in yeast (Sharma et al., Eukaryot. Cell 1(3), 2002, pp. 391-400). The C-terminally tagged T4_Fungal_5 protein showed a different GFP localization, consistent with the protein being localized to the plasma membrane (FIG. 11).

### Example 14: Directed Evolution of T4_Fungal_5 Protein Using Error-Prone PCR and Growth Selection

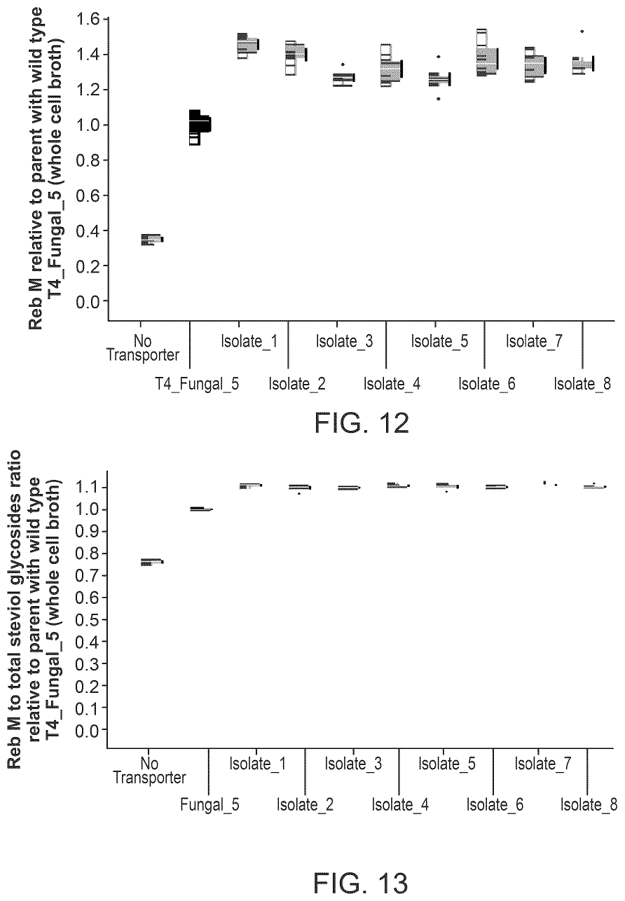

The transporter T4_Fungal_5 actively removes both Reb D and Reb M from the cytoplasm (see FIG. 4). Reb D is the immediate substrate for Reb M (FIG. 2), thus removing Reb D from the cytosol reduces the overall amount of Reb M produced by the yeast. T4_Fungal_5 was therefore subjected to enzyme evolution to increase both its overall activity and its specificity for Reb M. The DNA coding sequence (CDS) of T4_Fungal_5 was subjected to mutagenesis via error-prone PCR using GeneMorph II Random Mutagenesis Kit (Agilent Technologies, Inc) and the resulting DNA library was transformed into a Reb M yeast strain similar to the one used in the transporter screen mentioned in Example 11 but having two additional copies of UGT76G1 both expressed under GAL1 promoters. An additional transformation using the wild type T4_Fungal_5 transporter was performed as a control. The transformations were performed as described in Example 1. After the overnight recovery, the cultures were transferred into production medium supplemented with the selective antibiotic for continued growth. The OD600 of the cultures were monitored and serial dilutions of the cultures with fresh antibiotic-containing production medium were performed to avoid carbon starvation. The culture was sampled daily for both glycerol stock archives and plated for individual colony formation on antibiotic containing YPD agar plates. The TSG and Reb M titers of 88 colonies from each daily sample were assessed and compared using methods described in Examples 6, 7, and 8. From this data, the time point which had highest percent of colonies producing TSG titers equal to or greater than that of the control strain (expressing wild type T4 Fungal 5) was identified. Additional colonies from this time point were plated from the glycerol stock and 900 colonies were picked and screened. The screen identified eight isolates that increased Reb M titers by 26% to 47% and increased the Reb M/TSG ratio by 10% over the control (FIGS. 12 and 13). Data in FIGS. 12 and 13 show that the mutations identified in the T4_Fungal_5 transporter increased both overall activity on steviol glycosides and specificity for Reb M.

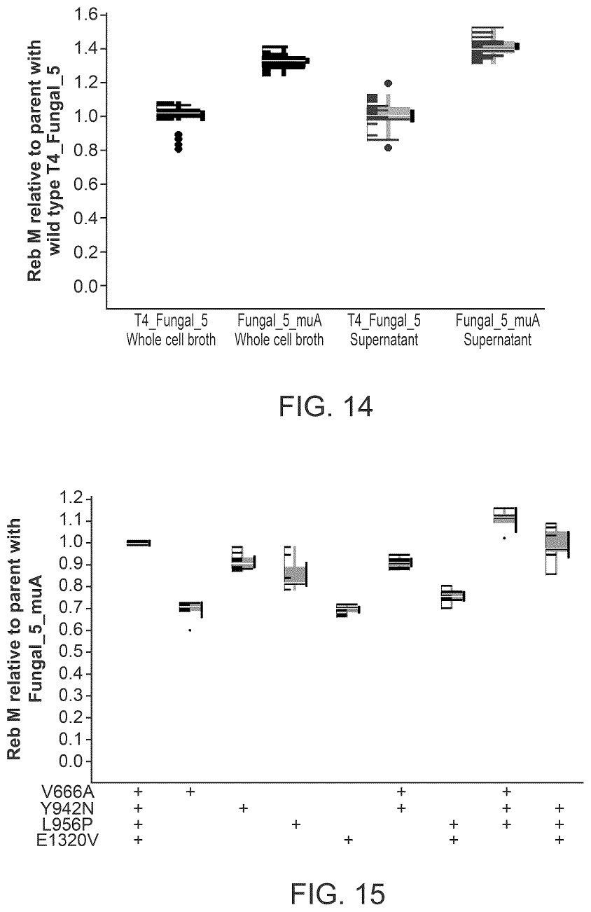

Sanger sequencing of the T4_Fungal_5 gene revealed that all eight isolates harbored the same nucleic acid substitutions, resulting in four amino acid substitutions: V666A, Y942N, L956P, and E1320V. This mutant allele was named “Fungal_5_muA”. To verify the causality of Fungal_5_muA on the improved titer and specificity, the mutant allele was amplified from one of the isolates and re-introduced into the parent strain. The resulting strain recapitulated the phenotypes and demonstrated the application of Fungal_5_muA in improvement of steviol glycoside production and specificity. When T4_Fungal_5 and Fungal_5_muA were expressed under the weaker GAL3 promoter, 30% more Reb M in whole cell broth and 40% more extracellular Reb M were produced by the strain with Fungal_5_muA than by the strain with the wild type T4_Fungal_5 (FIG. 14), consistent with earlier data.

### Example 15: Further Improvement of Fungal_5_muA

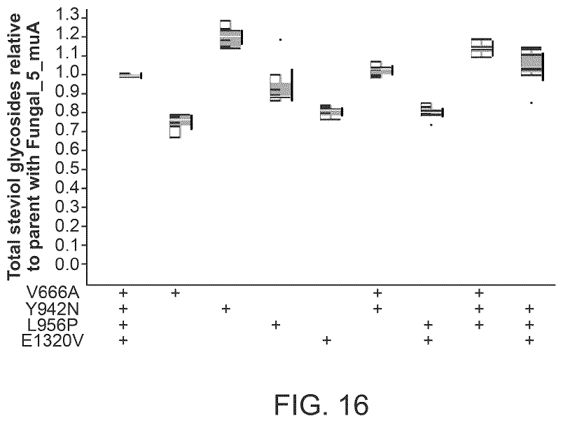

To further improve Fungal_5_muA by removing potentially detrimental mutations, we created additional T4_Fungal_5 mutant variants with either one, two, or three amino acid substitutions identified in Fungal_5_muA and introduced them into the yeast strain used for screening the mutagenesis library of T4_Fungal_5 in Example 14. Although single reversion of V666A in Fungal_5_muA had negligible impacts on either TSG or Reb M production, reversion of E1320V was beneficial and the V666A Y942N L956P triple mutant produced 14% more TSG and 12% more Reb M than the Fungal_5_muA strain (FIGS. 15 and 16). Further reversion of L956P in the triple mutant (V666A Y942N), however, led to 10% decrease in Reb M and 19% decrease in TSG produced as compared to the V666A Y942N L956P triple mutant. Compared to the Fungal_5_muA strain, the single Y942N mutant strain produced 21% more TSG but 10% lower amounts of Reb M. These data demonstrate that the Y942N mutation benefitted overall activity of T4_Fungal_5 in exporting steviol glycosides but had negative effect on its specificity for Reb M.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A genetically modified host cell capable of producing an isoprenoid compound comprising a heterologous nucleic acid encoding an ABC-transporter comprising an amino acid sequence having at least 90% sequence identity to an amino acid sequence selected from the group consisting of SEQ ID NO: 2, SEQ ID NO: 3, SEQ ID NO: 5, SEQ ID NO: 7, SEQ ID NO: 8, SEQ ID NO: 28, SEQ ID NO: 29, and SEQ ID NO: 30.

2. (canceled)

3. The genetically modified host cell of claim 1, further comprising a nucleic acid encoding geranylgeranyl pyrophosphate synthase (GGPPS), ent-copalyl pyrophosphate synthase (CPS), ent-kaurene synthase (KS), ent-kaurene 19-oxidase (KO), ent-kaurenoic acid 13-hydroxylase (KAH), cytochrome p450 reductase (CPR), and one or more UDP-glucosyltransferases (UGT).

4. The genetically modified host cell of claim 3, wherein the one or more UDP-glucosyltransferases (UGT) are selected from the group consisting of UGT85C2, UGT74G1, UGT91D_like3, UGT76G1, EUGT11, and UGT40087.

5. The genetically modified host cell of claim 4, wherein the geranylgeranyl pyrophosphate synthase (GGPPS) comprises an amino acid sequence having at least 90% sequence identity to SEQ ID NO: 9, the ent-copalyl pyrophosphate synthase (CPS) comprises an amino acid sequence having at least 90% sequence identity to SEQ ID NO: 10, the ent-kaurene synthase (KS) comprises an amino acid sequence having at least 80% sequence identity to SEQ ID NO: 11, the ent-kaurene 19-oxidase (KO) comprises an amino acid sequence having at least 90% sequence identity to SEQ ID NO: 12, the ent-kaurenoic acid 13-hydroxylase (KAH) comprises an amino acid sequence having at least 90% sequence identity to SEQ ID NO: 13, the cytochrome p450 reductase (CPR) comprises an amino acid sequence having at least 90% sequence identity to SEQ ID NO: 14, and the one or more UDP-glucosyltransferases (UGT) comprise an amino acid sequence having at least 90% sequence identity to an amino acid sequence selected from the group consisting of SEQ ID NO: 15, SEQ ID NO: 16, SEQ ID NO: 17, SEQ ID NO:18, SEQ ID NO: 19.

6. The genetically modified host cell of claim 5, wherein the geranylgeranyl pyrophosphate synthase (GGPPS) comprises an amino acid sequence of SEQ ID NO: 9, the ent-copalyl pyrophosphate synthase (CPS) comprises an amino acid sequence of SEQ ID NO: 10, the ent-kaurene synthase (KS) comprises an amino acid sequence of SEQ ID NO: 11, the ent-kaurene 19-oxidase (KO) comprises an amino acid sequence of SEQ ID NO: 12, the ent-kaurenoic acid 13-hydroxylase (KAH) comprises an amino acid sequence of SEQ ID NO: 13, the cytochrome p450 reductase (CPR) comprises an amino acid sequence of SEQ ID NO: 14, and the one or more UDP-glucosyltransferases (UGT) comprise an amino acid sequence selected from the group consisting of SEQ ID NO: 15, SEQ ID NO: 16, SEQ ID NO: 17, SEQ ID NO:18, SEQ ID NO: 19.

7. The genetically modified host cell of claim 1, wherein the host cell is selected from a bacterial cell, a fungal cell, an algal cell, an insect cell, and a plant cell.

8. The genetically modified host cell of claim 7, wherein the host cell is a Saccharomyces cerevisiae cell.

9. (canceled)

10. (canceled)

11. (canceled)

12. (canceled)

13. (canceled)

14. (canceled)

15. The genetically modified host cell of claim 1, wherein the ABC-transporter comprises an amino acid sequence having:
(i) at least 90% sequence identity to an amino acid sequence selected from the group consisting of SEQ ID NO: 2, SEQ ID NO: 7, SEQ ID NO: 29, and SEQ ID NO: 30; and
(ii) one or more amino acid substitutions selected from V666A, Y942N, L956P, and E1320V.

16. The genetically modified host cell of claim 15, wherein the ABC-transporter comprises an amino acid sequence selected from the group consisting of SEQ ID NO: 7, SEQ ID NO: 29, and SEQ ID NO: 30 having one or more amino acid substitutions selected from V666A, Y942N, L956P, and E1320V.

17. The genetically modified host cell of claim 16, further comprising a nucleic acid encoding geranylgeranyl pyrophosphate synthase (GGPPS) comprising an amino acid sequence of SEQ ID NO: 9.

18. (canceled)

19. (canceled)

20. (canceled)

21. (canceled)

22. The genetically modified host cell of claim 1, wherein the one or more steviol glycosides is selected from the group consisting of Reb A, Reb B, Reb D, Reb E, and Reb M.

23. (canceled)

24. (canceled)

25. (canceled)

26. (canceled)

27. (canceled)

28. (canceled)

29. (canceled)

30. The genetically modified host cell of claim 1, further comprising an UDP-glucosyltransferase (UGT) having an amino acid sequence having at least 90% sequence identity to the amino acid sequence of SEQ ID NO: 18.

31. The genetically modified host cell of claim 1, further comprising an UDP-glucosyltransferase (UGT) having an amino acid sequence of SEQ ID NO: 18.

32. (canceled)

33. (canceled)

34. (canceled)

35. The genetically modified host cell of claim 1, wherein the isoprenoid compound is selected from farnesyl pyrophosphate (FPP) and geranylgeranyl pyrophosphate (GGPP).

36. (canceled)

37. (canceled)

38. (canceled)

39. The genetically modified host cell of claim 1, wherein the isoprenoid compound is farnesyl pyrophosphate (FPP).

40. The genetically modified host cell of claim 1 wherein the isoprenoid compound is geranylgeranyl pyrophosphate (GGPP).

41. (canceled)

42. (canceled)

43. (canceled)

44. (canceled)

45. (canceled)

46. (canceled)

47. A method for producing an isoprenoid compound comprising the steps:
(a) culturing a population of the host cells of claim 1 in a medium with a carbon source under conditions suitable for making the isoprenoid compound to yield a culture broth; and
(b) recovering the isoprenoid compound from the culture broth.

48. The method of claim 47, wherein the isoprenoid compound is selected from farnesyl pyrophosphate (FPP) and geranylgeranyl pyrophosphate (GGPP).

49. The method of claim 47, wherein the isoprenoid compound is farnesyl pyrophosphate (FPP).

50. The method of claim 47, wherein the isoprenoid compound is geranylgeranyl pyrophosphate (GGPP).


SEQUENCE LISTING (30 sequences)

SEQ ID NO: 1 - protein, 1559 aa, synthetic construct
  REGION 1..1559: note = Synthetic: CEN.PK.BPT1 ABC-transporter
MSSLEVVDGCPYGYRPYPDSGTNALNPCFISVISAWQAVFFLLIGSYQLWKLYKNNKVPPRFKNFPTLPSKINSRHLTHLTNVCFQSTLIICELALVSQSSDRVYPFILKKALYLNLLFNLGISLPTQYLAYFKSTFSMGNQLFYYMFQILLQLFLILQRYYHGSSNERLTVISGQTAMILEVLLLFNSVAIFIYDLCIFEPINELSEYYKKNGWYPPVHVLSYITFIWMNKLIVETYRNKKIKDPNQLPLPPVDLNIKSISKEFKANWELEKWLNRNSLWRAIWKSFGRTISVAMLYETTSDLLSVVQPQFLRIFIDGLNPETSSKYPPLNGVFIALTLFVISVVSVFLTNQFYIGIFEAGLGIRGSLASLVYQKSLRLTLAERNEKSTGDILNLMSVDVLRIQRFFENAQTIIGAPIQIIVVLTSLYWLLGKAVIGGLVTMAIMMPINAFLSRKVKKLSKTQMKYKDMRIKTITELLNAIKSIKLYAWEEPMMARLNHVRNDMELKNFRKIGIVSNLIYFAWNCVPLMVTCSTFGLFSLFSDSPLSPAIVFPSLSLFNILNSAIYSVPSMINTIIETSVSMERLKSFLLSDEIDDSFIERIDPSADERALPAIEMNNITFLWKSKEVLTSSQSGDNLRTDEESIIGSSQIALKNIDHFEAKRGDLVCVVGRVGAGKSTFLKAILGQLPCMSGSRDSIPPKLIIRSSSVAYCSQESWIMNASVRENILFGHKFDQDYYDLTIKACQLLPDLKILPDGDETLVGEKGISLSGGQKARLSLARAVYSRADIYLLDDILSAVDAEVSKNIIEYVLIGKTALLKNKTIILTTNTVSILKHSQMIYALENGEIVEQGNYEDVMNRKNNTSKLKKLLEEFDSPIDNGNESDVQTEHRSESEVDEPLQLKVTESETEDEVVTESELELIKANSRRASLATLRPRPFVGAQLDSVKKTAQKAEKTEVGRVKTKIYLAYIKACGVLGVVLFFLFMILTRVFDLAENFWLKYWSESNEKNGSNERVWMFVGVYSLIGVASAAFNNLRSIMMLLYCSIRGSKKLHESMAKSVIRSPMTFFETTPVGRIINRFSSDMDAVDSNLQYIFSFFFKSILTYLVTVILVGYNMPWFLVFNMFLVVIYIYYQTFYIVLSRELKRLISISYSPIMSLMSESLNGYSIIDAYDHFERFIYLNYEKIQYNVDFVFNFRSTNRWLSVRLQTIGATIVLATAILALATMNTKRQLSSGMVGLLMSYSLEVTGSLTWIVRTTVTIETNIVSVERIVEYCELPPEAQSINPEKRPDENWPSKGGIEFKNYSTKYRENLDPVLNNINVKIEPCEKVGIVGRTGAGKSTLSLALFRILEPTEGKIIIDGIDISDIGLFDLRSHLAIIPQDAQAFEGTVKTNLDPFNRYSEDELKRAVEQAHLKPHLEKMLHSKPRGDDSNEEDGNVNDILDVKINENGSNLSVGQRQLLCLARALLNRSKILVLDEATASVDMETDKIIQDTIRREFKDRTILTIAHRIDTVLDSDKIIVLDQGSVREFDSPSKLLSDKTSIFYSLCEKGGYLK

SEQ ID NO: 2 - protein, 1620 aa, synthetic construct
  REGION 1..1620: note = Synthetic: T4_Fungal_1 ABC-transporter
MSLELSNSTLCDSYWAVDDFTACGRQLVESWVSVPLVLSALVVAFNLLRNSLASEKTDPYSKLDAEQQPLLQNGHALYTSSIESDNTDIFQRHFDIALLKPVKDDGKPIGVVRIVYRDTAEKLKVALEEILLISQTVLAFLALSRLEDISESRFLLVKYINFSLWLYLTVITSARLLNVTKGFSANRVDLWYHCAILYNLQWFNSVMLFRSALLHHVSGTYGYWFYVTQFVINTLLCLTNGLEKLSDKPAIVYEEEGVIPSPETTSSLIDIMTYGYLDKMVFSSYWKPITMEEVWGLRYDDYSHDVLIRFHKLKSSIRFTLRLFLQFKKELALQTLCTCIEALLIFVPPLCLKKILEYIESPHTKSRSMAWFYVLIMFGSGVIACSFSGRGLFLGRRICTRMRSILIGEIYSKALRRRLGSTDKEKTTEEEDDKSAKSKKEDEPSNKELGGIINLMAVDAFKVSEIGGYLHYFPNSFVMAAVAIYMLYKLLGWSSLIGTATLIAILPINYMLVEKLSKYQKQMLLVTDKRIQKTNEAFQNIRIIKYFAWEDKFADTIMKIREEELGYLVGRCVVWALLIFLWLVVPTIVTLITFYAYTVIQGNPLTSPIAFTALSLFTLLRGPLDALADMLSMVMQCKVSLDRVEDFLNEPETTKYQQLSAPRGPNSPLIGFENATFYWSKNSKAEFALKDLNIDFKVGKLNVVIGPTGSGKSSLLLALLGEMDLDKGNVFLPGAIPRDDLTPNPVTGLMESVAYCSQTAWLLNATVKDNIIFASPFNQERYDAVIHACGLTRDLSILEAGDETEIGEKGITLSGGQKQRVSLARALYSSASYLLLDDCLSAVDSHTAVHIYDYCINGELMKGRTCILVSHNVSLTVKEADFVVMMDNGRIKAQGSVDELMQEGLLNEEVVKSVMQSRSASTANLAALDDNSPISSEAIAEGLAKKTQKPEQSKKSKLIEDETKSDGSVKPEIYYAYFRYFGNPALWIMIAFLFIGSQSVNVYQSYWLRRWSAIEDKRDLSAFSNSNDMTLFLFPTFHSINWHRPLVNYALQPFGLAVEERSTMYYITIYTLIGLAFATLGSSRVILTFIGGLNVSRKIFKDLLDKLLHAKLRFFDQTPIGRIMNRFSKDIEAIDQELALYAEEFVTYLISCLSTLVVVCAVTPAFLVAGVLILLVYYGVGVLYLELSRDLKRFESITKSPIHQHFSETLVGMTTIRAYGDERRFLKQNFEKIDVNNRPFWYVWVNNRWLAYRSDMIGAFIIFFAAAFAVAYSDKIDAGLAGISLSFSVSFRYTAVWVVRMYAYVEMSMNSVERVQEYIEQTPQEPPKYLPQDPVNSWPSNGVIDVQNICIRYSPELPRVIDNVSFHVNAGEKIGVVGRTGAGKSTIITSFFRFVDLESGSIKIDGLDISKIGLKPLRKGLTIIPQDPTLFSGTIRSNLDIFGEYGDLQMFEALRRVNLISVDDYQRIVDGNGAAVADETAQARGDNVNKFLDLDSTVSEGGGNLSQGERQLLCLARSILKMPKILMLDEATASIDYESDAKIQATIREEFSSSTVLTIAHRLKTIIDYDKILLLDHGKVKEYDHPYKLITNKKSDFRKMCQDTGEFDDLVNLAKQAYRK

SEQ ID NO: 3 - protein, 1515 aa, synthetic construct
  REGION 1..1515: note = Synthetic: T4_Fungal_10 ABC-transporter
MGQSERAALIAFASRNTTECWLCRDKEGFGPISYYGDFTVCFIDGVLLNFAALFMLIFGTYQVVKLSKKEHPGIKYRRDWLLFSRITLVGCFLLFTSMAAYYSSEKHESIALTSQYTLTLMSIFVALMLHWVEYHRSRISNGIVLFYWLFETLFQGSKWVNFSIRHAYNLNHEWPVSYSVYILTIFQTISAFMILILEAGFEKPLPSYQRVIESYSKQKRNPVDNSHIFQRLSFSWMTELMKTGYKKYLTEQDLYKLPKSFGAKEISHKFSERWQYQLKHKANPSLAWALLSTFGGKILLGGIFKVAYDILQFTQPQLLRILIKFVSDYTSTPEPQLPLVRGVMLSIAMFVVSVVQTSILHQYFLNAFDTGMHIKSGMTSVIYQKALVLSSEASASSSTGDIVNLMSVDVQRLQDLTQWGQIIWSGPFQIILCLVSLYKLLGPCMWVGVIIMIIMIPINSVIVRIQKKLQKIQMKNKDERTRVTSEILNNIKSLKVYGWEIPYKAKLDHVRNDKELKNLKKMGCTLALASFQFNIVPFLVSCSTFAVFVFTEDRPLSTDLVFPALTLFNLLSFPLAVVPNAISSFIEASVSVNRLYAFLTNEELQTDAVHREPKVNNIGDEGVKVSDATFLWQRKPEYKVALKNINFSAKKGELTCIVGKVGSGKSALIQSLLGDLIRVKGYAAVHGSVAYVSQVAWIMNGTVKDNIIFGHKYDPEFYELTIKACALAIDLSMLPDGDQTLVGEKGISLSGGQKARLSLARAVYARADTYLLDDPLAAVDEHVAKHLIEHVLGPHGLLHSKTKVLATNKISVLSIADSITLMENGEIIQQGTYEETNNTTDSPLSKLISEFGKKGKATPSQSTTSLTKLATSDLGSSSDSKVSDVSIDVSQLDTENLTEAEELKSLRRASMATLGSIGFDDDENIARREHREQGKVKWDIYMEYARACNPRSVCVFLFFIVLSMLLSVLGNFWLKHWSEVNTGEGYNPHAARYLLIYFALGVGSALATLIQTIVLWVFCTIHGSRYLHDAMATSVLKAPMSFFETTPIGRILNRFSNDIYKVDEVLGRTFSQFFANVVKVSFTIIVICMATWQFIFIILPLSVLYIYYQQYYLRTSRELRRLDSVTRSPIYAHFQETLGGLTTIRGYSQQTRFVHINQTRVDNNMSAFYPSVNANRWLAFRLEFIGSIIILGSSMLAVIRLGNGTLTAGMIGLSLSFALQITQSLNWIVRMTVEVETNIVSVERIKEYAELKSEAPYIIEDHRPPASWPEKGDVKFVNYSTRYRPELELILKDINLHILPKEKIGIVGRTGAGKSSLTLALFRIIEAASGHIIIDGIPIDSIGLADLRHRLSIIPQDSQIFEGTIRENIDPSKQYTDEQIWDALELSHLKNHVKNMGPDGLETMLSEGGGNLSVGQRQLMCLARALLISSKILVLDEATAAVDVETDQLIQKTIREAFKERTILTIAHRINTIMDSDRIIVLDKGRVTEFDTPANLLNKKDSIFYSLCVEAGLAE

SEQ ID NO: 4 - protein, 1559 aa, synthetic construct
  REGION 1..1559: note = Synthetic: T4_Fungal_2 ABC-transporter
MSSLEVVDGCPYGYRPYPDSGTNALNPCFISVISAWQAVFFLLIGSYQLWKLYKNNKVPPRFKNFPTLPSKINSRHLTHLTNVCFQSTLIICELALVSQSSDRVYPFILKKALYLNLLFNLGISLPTQYLAYFKSTFSMGNQLFYYMFQILLQLFLILQRYYHGSSNERLTVISGQTAMILEVLLLFNSVAIFIYDLCIFEPINELSEYYKKNGWYPPVHVLSYITFIWMNKLIVETYRNKKIKDPNQLPLPPVDLNIKSISKEFKANWELEKWLNRNSLWRAIWKSFGRTISVAMLYETTSDLLSVVQPQFLRIFIDGFNPETSSKYPPLNGVFIALTLFVISVVSVFLTNQFYIGIFEAGLGIRGSLASLVYQKSLRLTLAERNEKSTGDILNLMSVDVLRIQRFFENAQTIIGAPIQIIVVLTSLYWLLGKAVVGGLVTMAIMMPINAFLSRKVKKLSKTQMKYKDMRIKTITELLNAIKSIKLYAWEEPMMARLNHVRNDMELKNFRKIGIVSNLIYFAWNCVPLMVTCSTFGLFSLFSDSPLSPAIVFPSLSLFNILNSAIYSVPSMINTIIETSVSMERLKSFLLSDEIDDSFIERIDPSADERALPAIEMNNITFLWKSKEVLASSQSGDNLRTDEESIIGSSQIALKNIDHFEAKRGDLVCVVGRVGAGKSTFLKAILGQLPCMSGSRDSIPPKLIIRSSSVAYCSQESWIMNASVRENILFGHKFDQNYYDLTIKACQLLPDLKILPDGDETLVGEKGISLSGGQKARLSLARAVYSRADIYLLDDILSAVDAEVSKNIIEYVLIGKTALLKNKTIILTTNTVSILKHSQMIYALENGEIVEQGNYEDVMNRKNNTSKLKKLLEEFDSPIDNGNESDVQTEHRSESEVDEPLQLKVTESETEDEVVTESELELIKANSRRASLATLRPRPFVGAQLDSVKKTAQEAEKTEVGRVKTKVYLAYIKACGVLGVVLFFLFMILTRVFDLAENFWLKYWSESNEKNGSNERVWMFVGVYSLIGVASAAFNNLRSIMMLLYCSIRGSKKLHESMAKSVIRSPMTFFETTPVGRIINRFSSDMDAVDSNLQYIFSFFFKSILTYLVTVILVGYNMPWFLVFNMFLVVIYIYYQTFYIVLSRELKRLISISYSPIMSLMSESLNGYSIIDAYDHFERFIYLNYEKIQYNVDFVFNFRSTNRWLSVRLQTIGATIVLATAILALATMNTKRQLSSGMVGLLMSYSLEVTGSLTWIVRTTVMIETNIVSVERIVEYCELPPEAQSINPEKRPDENWPSKGGIEFKNYSTKYRENLDPVLNNINVKIEPCEKVGIVGRTGAGKSTLSLALFRILEPTEGKIIIDGIGISDIGLFDLRSHLAIIPQDAQAFEGTVKTNLDPFNRYSEDELKRAVEQAHLKPHLEKMLHSKPRGDDSNEEDGNVNDILDVKINENGSNLSVGQRQLLCLARALLNRSKILVLDEATASVDMETDKIIQDTIRREFKDRTILTIAHRIDTVLDSDKIIVLDQGSVREFDSPSKLLSDKTSIFYSLCEKGGYLK

SEQ ID NO: 5 - protein, 1638 aa, synthetic construct
  REGION 1..1638: note = Synthetic: T4_Fungal_3 ABC-transporter
MNSYNESAPTGCSFWDNDDISPCIRKSLLDSYLPAAIVVGSLLYLLLIGAQQIKTHRKLYAKDETQPLLEPANGSPTDYSNTYGTIDYEEEQSTAELTTSQKHFDISRLEPLKDDGTPLGLVKYVQRDGWEKVKLILEFVILIFQLVIAVVALFVPSLNQEWEGYKLTPIVRVFVWIFLFALGSIRALNKSGPFPLANISLLYYIVNIVPSALSFRSVLIHPQNSQLVNYYYSFQFINNTLLFLLLGSARVFDHPSVLFDTDDGVKPSPENNSNFFEIVTYSWIDPLIFKAYKTPLQFNDIWGLRIDDYAYFLLRRFKDLGFTRTFTYKIFYFSKGDLAAQALWASIDSMLIFGPSLLLKRILEYVDNPGMTSRNMAWLYVLTMFFIQISDSLVSGRSLYLGRRVCIRMKALIIGEVYAKALRRRMTSPEELIEEVDPKDGKAPIADQTSKEESKSTELGGIINLMAVDASKVSELCSYLHFFVNSFFMIIVAVTLLYRLLGWSALAGSSSILILLPLNYKLASKIGEFQKEMLGITDNRIQKLNEAFQSIRIIKFFAWEENFAKEIMKVRNEEIRYLRYRVIVWTCSAFVWFITPTLVTLISFYFYVVFQGKILTTPVAFTALSLFNLLRSPLDQLSDMLSFMVQSKVSLDRVQKFLEEQESDKYEQLTHTRGANSPEVGFENATLSWNKGSKNDFQLKDIDIAFKVGKLNVIIGPTGSGKTSLLLGLLGEMQLTNGKIFLPGSTPRDELIPNPETGMTEAVAYCSQIAWLLNDTVKNNIVFAAPFNQQRYDAVIDACGLTRDLKVLDAGDATEIGEKGITLSGGQKQRVSLARALYSNARHVLLDDCLSAVDSHTAAWIYENCITGPLMKDRTCILVSHNVALTVRDAAWIVAMDNGRVLEQGTCEDLLSSGSLGHDDLVSTVISSRSQSSVNLKQLNVSDTSEIHQKLKKIAESDKADQLDEERLSPRGKLIEDETKSSGAVSWEVYKFYGRAFGGVFIWFVFVAAFAASQGSNIMQSVWLKIWAAANDKLVSPAFTMSIDRSLNALKEGFRASVASVEWSRPLGGEMFRVYGEESSHSSGYYITIYALIGLSYALISAFRVYVVFMGGIVASNKIFEDMLTKIFNAKLRFFDSTPIGRIMNRFSKDTESIDQELAPYAEGFIVSVLQCGATILLICIITPGFIVFAAFIVIIYYYIGALYLASSRELKRYDSITVSPIHQHFSETLVGVTTIRAYGDERRFMRQNLEKIDNNNRSFFYLWVANRWLALRVDFVGALVSLLSAAFVMLSIGHIDAGMAGLSLSYAIAFTQSALWVVRLYSVVEMNMNSVERLEEYLNIDQEPDREIPDNKPPSSWPETGEIEVDDVSLRYAPSLPKVIKNVSFKVEPRSKIGIVGRTGAGKSTIITAFFRFVDPESGSIKIDGIDITSIGLKDLRNAVTIIPQDPTLFTGTIRSNLDPFNQYSDAEIFESLKRVNLVSTDEPTSGSSSDNIEDSNENVNKFLNLNNTVSEGGSNLSQGQRQLTCLARSLLKSPKIILLDEATASIDYNTDSKIQTTIREEFSDSTILTIAHRLRSIIDYDKILVMDAGRVVEYDDPYKLISDQNSLFYSMCSNSGELDTLVKLAKEAFIAKRNKK

SEQ ID NO: 6 - protein, 1559 aa, synthetic construct
  REGION 1..1559: note = Synthetic: T4_Fungal_4 ABC-transporter
MSSLEVVDGCPYGYRPYPDSGTNALNPCFISVISAWQAVFFLLIGSYQLWKLYKNNKVPPRFKNFPTLPSKINSRHLTHLTNVCFQSTLIICELALVSQSSDRVYPFILKKALYLNLLFNLGISLPTQYLAYFKSTFSMGNQLFYYMFQILLQLFLILQRYYHGSSNERLTVISGQTAMILEVLLLFNSVAIFIYDLCIFEPINELSEYYKKNGWYPPVHVLSYITFIWMNKLIVETYRNKKIKDPNQLPLPPVDLNIKSISKEFKANWELEKWLNRNSLWRAIWKSFGRTISVAMLYETTSDLLSVVQPQFLRIFIDGFNPETSSKYPPLNGVFIALTLFVISVVSVFLTNQFYIGIFEAGLGIRGSLASLVYQKSLRLTLAERNEKSTGDILNLMSVDVLRIQRFFENAQTIIGAPIQIIVVLTSLYWLLGKAVIGGLVTMAIMMPINAFLSRKVKKLSKTQMKYKDMRIKTITELLNAIKSIKLYAWEEPMMARLNHVRNDMELKNFRKIGIVSNLIYFAWNCVPLMVTCSTFGLFSLFSDSPLSPAIVFPSLSLFNILNSAIYSVPSMINTIIETSVSMERLKSFLLSDEIDDSFIERIDPSADERALPAIEMNNITFLWKSKEVLASSQSRDNLRTDEESIIGSSQIALKNIDHFEAKRGDLVCVVGRVGAGKSTFLKAILGQLPCMSGSRDSIPPKLIIRSSSVAYCSQESWIMNASVRENILFGHKFDQNYYDLTIKACQLLPDLKILPDGDETLVGEKGISLSGGQKARLSLARAVYSRADIYLLDDILSAVDAEVSKNIIEYVLIGKTALLKNKTIILTTNTVSILKHSQMIYALENGEIVEQGNYEDVMNRKNNTSKLKKLLEEFDSPIDNGNESDVQTEHRSESEVDEPLQLKVTESETEDEVVTESELELIKANSRRASLATLRPRPFVGAQLDSVKKTAQEAEKTEVGRVKTKVYLAYIKACGVLGVVLFFLFMILTRVFDLAENFWLKYWSESNEKNGSNERVWMFVGVYSLIGVASAAFNNLRSIMMLLYCSIRGSKKLHESMAKSVIRSPMTFFETTPVGRIINRFSSDMDAVDSNLQYIFSFFFKSILTYLVTVILVGYNMPWFLVFNMFLVVIYIYYQTFYIVLSRELKRLISISYSPIMSLMSESLNGYSIIDAYDHFERFIYLNYEKIQYNVDFVFNFRSTNRWLSVRLQTIGATIVLATAILALATMNTKRQLSSGMVGLLMSYSLEVTGSLTWIVRTTVMIETNIVSVERIVEYCELPPEAQSINPEKRPDENWPSKGGIEFKNYSTKYRENLDPVLNNINVKIEPCEKVGIVGRTGAGKSTLSLALFRILEPTEGKIIIDGIDISDIGLFDLRSHLAIIPQDAQAFEGTVKTNLDPFNRYSEDELKRAVEQAHLKPHLEKMLHSKPRGDDSNEEDGNVNDILDVKINENGSNLSVGQRQLLCLARALLNRSKILVLDEATASVDMETDKIIQDTIRREFKDRTILTIAHRIDTVLDSDKIIVLDQGSVREFDSPSKLLSDKTSIFYSLCEKGGYLK

SEQ ID NO: 7 - protein, 1381 aa, synthetic construct
  REGION 1..1381: note = Synthetic: T4_Fungal_5 ABC-transporter
MTSPGSEKCTPRSDEDLERSEPQLQRRLLTPFLLSKKVPPIPKEDERKPYPYLKTNPLSQILFWWLNPLLRVGYKRTLDPNDFYYLEHSQDIETTYSNYEMHLARILEKDRAKARAKDPTLTDEDLKNREYPKNAVIKALFLTFKWKYLWSIFLKLLSDIVLVLNPLLSKALINFVDEKMYNPDMSVGRGVGYAIGVTFMLGTSGILINHFLYLSLTVGAHCKAVLTTAIMNKSFRASAKSKHEYPSGRVTSLMSTDLARIDLAIGFQPFAITVPVPIGVAIALLIVNIGVSALAGIAVFLVCIVVISASSKSLLKMRKGANQYTDARISYMREILQNMRIIKFYSWEDAYEKSVVTERNSEMSIILKMQSIRNFLLALSLSLPAIISMVAFLVLYGVSNDKNPGNIFSSISLFSVLAQQTMMLPMALATGADAKIGLERLRQYLQSGDIEKEYEDHEKPGDRDVVLPDNVAVELNNASFIWEKFDDADDNDGNSEKTKEVVVTSKSSLTDSSHIDKSTDSADGEYIKSVFEGFNNINLTIKKGEFVIITGPIGSGKSSLLVALAGFMKKTSGTLGVNGTMLLCGQPWVQNCTVRDNILFGLEYDEARYDRVVEVCALGDDLKMFTAGDQTEIGERGITLSGGQKARINLARAVYANKDIILLDDVLSAVDARVGKLIVDDCLTSFLGDKTRILATHQLSLIEAADRVIYLNGDGTIHIGTVQELLESNEGFLKLMEFSRKSESEDEEDVEAANEKDVSLQKAVSVVQEQDAHAGVLIGQEERAVNGIEWDIYKEYLHEGRGKLGIFAIPTIIMLLVLDVFTSIFVNVWLSFWISHKFKARSDGFYIGLYVMFVILSVIWITAEFVVMGYFSSTAARRLNLKAMKRVLHTPMHFLDVTPMGRILNRFTKDTDVLDNEIGEQARMFLHPAAYVIGVLILCIIYIPWFAIAIPPLAILFTFITNFYIASSREVKRIEAIQRSLVYNNFNEVLNGLQTLKAYNATSRFMEKNKRLLNRMNEAYLLVIANQRWISVNLDLVSCCFVFLISMLSVFRVFDINASSVGLVVTSVLQIGGLMSLIMRAYTTVENEMNSVERLCHYANKLEQEAPYIMNETKPRPTWPEHGAIEFKHASMRYREGLPLVLKDLTISVKGGEKIGICGRTGAGKSTIMNALYRLTELAEGSITIDGVEISQLGLYDLRSKLAIIPQDPVLFRGTIRKNLDPFGQNDDETLWDALRRSGLVEGSILNTIKSQSKDDPNFHKFHLDQTVEDEGANFSLGERQLIALARALVRNSKILILDEATSSVDYETDSKIQKTISTEFSHCTILCIAHRLKTILTYDRILVLEKGEVEEFDTPRVLYSKNGVFRQMCERSEITSADFV

SEQ ID NO: 8 - protein, 1650 aa, synthetic construct
  REGION 1..1650: note = Synthetic: T4_Fungal_8 ABC-transporter
MSGSNSNSNLDAISDSCPFWRYDDITECGRVQYINYYLPITLVGVSLLYLFKNAIQHYYRKPQEIKPSVASELLGSNLTDLPNENKPLLSESTQALYTNPDSNKTGFSLKEEHFSINKVTLTEIHSNKHDAVKIVRRNWLEKLRVFLEWVLCALQLCIYISVWSKYTNTQEDFPMHASISGLMLWSLLLLVVSLRLANINQNISWINSGPGNLWALSFACYLSLFCGSVLPLRSIYIGHITDEIASTFYKLQFYLSLTLFLLLFTSQAGNRFAIIYKSTPDITPSPEPIVSIASYITWAWVDKFLWKAHQNYIEMKDVWGLMVEDYSILVIKRFNHFVQNKTKSRTFSFNLIHFFMKFIAIQGAWATISSVISFVPTMLLRRILEYVEDQSTAPLNLAWMYIFLMFLARILTAICAAQALFLGRRVCIRMKAIIISEIYSKALRRKISPNSTKEPTDVVDPQELNDKQHVDGDEESATTANLGAIINLMAVDAFKVSEICAYLHSFIEAIIMTIVALFLLYRLIGWSALVGSAMIICFLPLNFKLASLLGTLQKKSLAITDKRIQKLNEAFQAIRIIKFFSWEENFEKDIQNTRDEELNMLLKRSIVWALSSLVWFITPSIVTSASFAVYIYVQGQTLTTPVAFTALSLFALLRNPLDMLSDMLSFVIQSKVSLDRVQEFLNEEETKKYEQLTVSRNKLGLQNATFTWDKNNQDFKLKNLTIDFKIGKLNVIVGPTGSGKTSLLMGLLGEMELLNGKVFVPSLNPREELVVEADGMTNSIAYCSQAAWLLNDTVRNNILFNAPYNENRYNAVISACGLKRDFEILSAGDQTEIGEKGITLSGGQKQRVSLARSLYSSSRHLLLDDCLSAVDSHTALWIYENCITGPLMEGRTCVLVSHNVALTLKNADWVIIMENGRVKEQGEPVELLQKGSLGDDSMVKSSILSRTASSVNISETNSKISSGPKAPAESDNANEESTTCGDRSKSSGKLIAEETKSNGVVSLDVYKWYAVFFGGWKMISFLCFIFLFAQMISISQAWWLRAWASNNTLKVFSNLGLQTMRPFALSLQGKEASPVTLSAVFPNGSLTTATEPNHSNAYYLSIYLGIGVFQALCSSSKAIINFVAGIRASRKIFNLLLKNVLYAKLRFFDSTPIGRIMNRFSKDIESIDQELTPYMEGAFGSLIQCVSTIIVIAYITPQFLIVAAIVMLLFYFVAYFYMSGARELKRLESMSRSPIHQHFSETLVGITTIRAFSDERRFLVDNMKKIDDNNRPFFYLWVCNRWLSYRIELIGALIVLAAGSFILLNIKSIDSGLAGISLGFAIQFTDGALWVVRLYSNVEMNMNSVERLKEYTTIEQEPSNVGALVPPCEWPQNGKIEVKDLSLRYAAGLPKVIKNVTFTVDSKCKVGIVGRTGAGKSTIITALFRFLDPETGYIKIDDVDITTIGLKRLRQSITIIPQDPTLFTGTLKTNLDPYNEYSEAEIFEALKRVNLVSSEELGNPSTSDSTSVHSANMNKFLDLENEVSEGGSNLSQGQRQLICLARSLLRCPKVILLDEATASIDYNSDSKIQATIREEFSNSTILTIAHRLRSIIDYDKILVMDAGEVKEYDHPYSLLLNRDSIFYHMCEDSGELEVLIQLAKESFVKKLNAN

SEQ ID NO: 9 - protein, 320 aa, synthetic construct
  REGION 1..320: note = Synthetic: Bt.GGPPS
MLTSSKSIESFPKNVQPYGKHYQNGLEPVGKSQEDILLEPFHYLCSNPGKDVRTKMIEAFNAWLKVPKDDLIVITRVIEMLHSASLLIDDVEDDSVLRRGVPAAHHIYGTPQTINCANYVYFLALKEIAKLNKPNMITIYTDELINLHRGQGMELFWRDTLTCPTEKEFLDMVNDKTGGLLRLAVKLMQEASQSGTDYTGLVSKIGIHFQVRDDYMNLQSKNYADNKGFCEDLTEGKFSFPIIHSIRSDPSNRQLLNILKQRSSSIELKQFALQLLENTNTFQYCRDFLRVLEKEAREEIKLLGGNIMLEKIMDVLSVNE

SEQ ID NO: 10 - protein, 736 aa, synthetic construct
  REGION 1..736: note = Synthetic: Ent-Os.CDPS
MEHARPPQGGDDDVAASTSELPYMIESIKSKLRAARNSLGETTVSAYDTAWIALVNRLDGGGERSPQFPEAIDWIARNQLPDGSWGDAGMFIVQDRLINTLGCVVALATWGVHEEQRARGLAYIQDNLWRLGEDDEEWMMVGFEITFPVLLEKAKNLGLDINYDDPALQDIYAKRQLKLAKIPREALHARPTTLLHSLEGMENLDWERLLQFKCPAGSLHSSPAASAYALSETGDKELLEYLETAINNFDGGAPCTYPVDNFDRLWSVDRLRRLGISRYFTSEIEEYLEYAYRHLSPDGMSYGGLCPVKDIDDTAMAFRLLRLHGYNVSSSVFNHFEKDGEYFCFAGQSSQSLTAMYNSYRASQIVFPGDDDGLEQLRAYCRAFLEERRATGNLRDKWVIANGLPSEVEYALDFPWKASLPRVETRVYLEQYGASEDAWIGKGLYRMTLVNNDLYLEAAKADFTNFQRLSRLEWLSLKRWYIRNNLQAHGVTEQSVLRAYFLAAANIFEPNRAAERLGWARTAILAEAIASHLRQYSANGAADGMTERLISGLASHDWDWRESNDSAARSLLYALDELIDLHAFGNASDSLREAWKQWLMSWTNESQGSTGGDTALLLVRTIEICSGRHGSAEQSLKNSEDYARLEQIASSMCSKLATKILAQNGGSMDNVEGIDQEVDVEMKELIQRVYGSSSNDVSSVTRQTFLDVVKSFCYVAHCSPETIDGHISKVLFEDVN

SEQ ID NO: 11 - protein, 757 aa, synthetic construct
  REGION 1..757: note = Synthetic: Ent-Pg.KS
MKREQYTILNEKESMAEELILRIKRMFSEIENTQTSASAYDTAWVAMVPSLDSSQQPQFPQCLSWIIDNQLLDGSWGIPYLIIKDRLCHTLACVIALRKWNAGNQNVETGLRFLRENIEGIVHEDEYTPIGFQIIFPAMLEEARGLGLELPYDLTPIKLMLTHREKIMKGKAIDHMHEYDSSLIYTVEGIHKIVDWNKVLKHQNKDGSLFNSPSATACALMHTRKSNCLEYLSSMLQKLGNGVPSVYPINLYARISMIDRLQRLGLARHFRNEIIHALDDIYRYWMQRETSREGKSLTPDIVSTSIAFMLLRLHGYDVPADVFCCYDLHSIEQSGEAVTAMLSLYRASQIMFPGETILEEIKTVSRKYLDKRKENGGIYDHNIVMKDLRGEVEYALSVPWYASLERIENRRYIDQYGVNDTWIAKTSYKIPCISNDLFLALAKQDYNICQAIQQKELRELERWFADNKFSHLNFARQKLIYCYFSAAATLFSPELSAARVVWAKNGVITTVVDDFFDVGGSSEEIHSFVEAVRVWDEAATDGLSENVQILFSALYNTVDEIVQQAFVFQGRDISIHLREIWYRLVNSMMTEAQWARTHCLPSMHEYMENAEPSIALEPIVLSSLYFVGPKLSEEIICHPEYYNLMHLLNICGRLLNDIQGCKREAHQGKLNSVTLYMEENSGTTMEDAIVYLRKTIDESRQLLLKEVLRPSIVPRECKQLHWNMMRILQLFYLKNDGFTSPTEMLGYVNAVIVDPIL

SEQ ID NO: 12 - protein, 499 aa, synthetic construct
  REGION 1..499: note = Synthetic: Ps.KO
MDTLTLSLGFLSLFLFLFLLKRSTHKHSKLSHVPVVPGLPVIGNLLQLKEKKPHKTFTKMAQKYGPIFSIKAGSSKIIVLNTAHLAKEAMVTRYSSISKRKLSTALTILTSDKCMVAMSDYNDFHKMVKKHILASVLGANAQKRLRFHREVMMENMSSKFNEHVKTLSDSAVDFRKIFVSELFGLALKQALGSDIESIYVEGLTATLSREDLYNTLVVDFMEGAIEVDWRDFFPYLKWIPNKSFEKKIRRVDRQRKIIMKALINEQKKRLTSGKELDCYYDYLVSEAKEVTEEQMIMLLWEPIIETSDTTLVTTEWAMYELAKDKNRQDRLYEELLNVCGHEKVTDEELSKLPYLGAVFHETLRKHSPVPIVPLRYVDEDTELGGYHIPAGSEIAINIYGCNMDSNLWENPDQWIPERFLDEKYAQADLYKTMAFGGGKRVCAGSLQAMLIACTAIGRLVQEFEWELGHGEEENVDTMGLTTHRLHPLQVKLKPRNRIY

SEQ ID NO: 13 - protein, 500 aa, synthetic construct
  REGION 1..500: note = Synthetic: Sr.KAH
MEASYLYISILLLLASYLFTTQLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRSSSSPVTLITVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVIGNNRLIDESDIGNIPYIGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 14 - protein, 711 aa, synthetic construct
  REGION 1..711: note = Synthetic: At.CPR
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 15 - protein, 481 aa, synthetic construct
  REGION 1..481: note = Synthetic: UGT85C2
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 16 - protein, 460 aa, synthetic construct
  REGION 1..460: note = Synthetic: UGT74G1
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 17 - protein, 485 aa, synthetic construct
  REGION 1..485: note = Synthetic: UGT91D_like3
MYNVTYHQNSKAMATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 18 - protein, 458 aa, synthetic construct
  REGION 1..458: note = Synthetic: UGT76G1
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 19 - protein, 436 aa, synthetic construct
  REGION 1..436: note = Synthetic: UGT40087
MDASDSSPLHIVIFPWLAFGHMLASLELAERLAARGHRVSFVSTPRNISRLRPVPPALAPLIDFVALPLPRVDGLPDGAEATSDIPPGKTELHLKALDGLAAPFAAFLDAACADGSTNKVDWLFLDNFQYWAAAAAADHKIPCALNLTFAASTSAEYGVPRVEPPVDGSTASILQRFVLTLEKCQFVIQRACFELEPEPLPLLSDIFGKPVIPYGLVPPCPPAEGHKREHGNAALSWLDKQQPESVLFIALGSEPPVTVEQLHEIALGLELAGTTFLWALKKPNGLLLEADGDILPPGFEERTRDRGLVAMGWVPQPIILAHSSVGAFLTHGGWASTIEGVMSGHPMLFLTFLDEQRINAQLIERKKAGLRVPRREKDGSYDRQGIAGAIRAVMCEEESKSVFAANAKKMQEIVSDRNCQEKYIDELIQRLGSFEK

SEQ ID NO: 20 - DNA, 4680 bp, synthetic construct
  misc_feature 1..4680: note = Synthetic: nucleic acid sequence
  source 1..4680: mol_type = other DNA
atgtcttcactagaagtggtagatgggtgcccctatggataccgaccatatccagatagtggcacaaatgcattaaatccatgttttatatcagtaatatccgcctggcaagccgtctttttcctattgattggtagctatcaattgtggaaactttataagaacaataaagtaccacccagatttaagaactttcctacattaccaagtaaaatcaacagtcgacatctaacgcatttgaccaatgtttgctttcagtccacgcttataatttgtgaactggccttggtatcccaatctagcgatagggtttatccatttatactaaagaaggctctgtacttgaatctccttttcaatttgggtatttctctccctactcaatacttagcttattttaaaagtacattttcaatgggcaaccagcttttctattacatgtttcaaattcttctacagctcttcttgatattgcagaggtactatcatggttctagtaacgaaaggcttactgttattagcggacaaactgctatgattttagaagtgctccttcttttcaattctgtggcaatttttatttatgatctatgcatttttgagccaattaacgaattatctgaatactacaagaaaaatgggtggtatccccccgttcatgtactatcctatattacatttatctggatgaacaaactgattgtggaaacttaccgtaacaagaaaatcaaagatcctaaccagttaccattgccgccagtagatctgaatattaagtcgataagtaaggaatttaaggctaactgggaattggaaaaatggttgaatagaaattctctttggagggccatttggaagtcatttggtaggactatttctgtggctatgctgtatgaaacgacatctgatttactttctgtagtacagccccagtttctacggatattcatagatggtttgaacccggaaacatcttctaaatatcctcctttaaatggtgtatttattgctctaacccttttcgtaatcagcgtggtttctgtgttcctcaccaatcaattttatattggaatttttgaggctggtttggggataagaggctctttagcttctttagtgtatcagaagtccttaagattgacgctagcagagcgtaacgaaaaatctactggtgacatcttaaatttgatgtctgtggatgtgttaaggatccagcggtttttcgaaaatgcccaaaccattattggcgctcctattcagattattgttgtattaacttccctgtactggttgctaggaaaggctgttattggagggttggttactatggctattatgatgcctatcaatgccttcttatctagaaaggtaaaaaagctatcaaaaactcaaatgaagtataaggacatgagaatcaagactattacagagcttttgaatgctataaaatctattaaattatacgcctgggaggaacctatgatggcaagattgaatcatgttcgtaatgatatggagttgaaaaattttcggaaaattggtatagtgagcaatctgatatattttgcgtggaattgtgtacctttaatggtgacatgttccacatttggcttattttctttatttagtgattctccgttatctcctgccattgtcttcccttcattatctttatttaatattttgaacagtgccatctattccgttccatccatgataaataccattatagagacaagcgtttctatggaaagattaaagtcattcctacttagtgacgaaattgatgattcgttcatcgaacgtattgatccttcagcggatgaaagagcgttacctgctatagagatgaataatattacatttttatggaaatcaaaagaagtattaacatctagccaatctggagataatttgaggacagatgaagagtctattatcggatcttctcaaattgcgttgaagaatatcgatcattttgaagcaaaaaggggtgatttagtttgtgttgttggtcgggtaggagctggtaaatcaacatttttgaaggcaattcttggtcaacttccttgcatgagtggttctagggactcgataccacctaaactgatcattagatcatcgtctgtagcctactgttcacaagaatcctggataatgaacgcatctgtaagagaaaacattctatttggtcacaagttcgaccaagattattatgacctcactattaaagcatgtcaattgctacccgatttgaaaatactaccagatggtgatgaaactttggtaggtgaaaagggcatttccctatcaggcggtcagaaggcccgtctttcattagccagagcggtgtactcgagagcagatatttatttgttggatgacattttatctgctgttgatgcagaagttagtaaaaatattattgaatatgttttgatcggaaagacggctttattaaaaaataaaacaattattttaactaccaatactgtatcaattttaaaacattcgcagatgatatatgcgctagaaaacggtgaaattgttgaacaagggaattatgaggatgtaatgaaccgtaagaacaatacttcaaaactgaaaaaattactagaggaatttgattctccgattgataatggaaatgaaagcgatgtccaaactgaacaccgatccgaaagtgaagtggatgaacctctgcagcttaaagtaactgaatcagaaactgaggatgaggttgttactgagagtgaattagaactaatcaaagccaattctagaagagcttctctagctacgctaagacctagaccctttgtgggagcacaattggattccgtgaagaaaacggcgcaaaaggccgagaagacagaggtgggaagagtcaaaacaaagatttatcttgcgtatattaaggcttgtggagttttaggtgttgttttatttttcttgtttatgatattaacaagggttttcgacttagcagagaatttttggttaaagtactggtcagaatctaatgaaaaaaatggttcaaatgaaagggtttggatgtttgttggtgtgtattccttaatcggagtagcatcggccgcattcaataatttacggagtattatgatgctactgtattgttctattaggggttctaagaaactgcatgaaagcatggccaaatctgtaattagaagtcctatgactttctttgagactacaccagttggaaggatcataaacaggttctcatctgatatggatgcagtggacagtaatctacagtacattttctccttttttttcaaatcaatactaacctatttggttactgttatattagtcgggtacaatatgccatggtttttagtgttcaatatgtttttggtggttatctatatttactatcaaacattttacattgtgctatctagggagctaaaaagattgatcagtatatcttactctccgattatgtccttaatgagtgagagcttgaacggttattctattattgatgcatacgatcattttgagagattcatctatctaaattatgaaaaaatccaatacaacgttgattttgtcttcaactttagatcaacgaatagatggttatccgtgagattgcaaactattggtgctacaattgttttggctactgcaatcttagcactagcaacaatgaatactaaaaggcaactaagttcgggtatggttggtctactaatgagctattcattagaggttacaggttcattgacttggattgtaaggacaactgtgacgattgaaaccaacattgtatcagtggagagaattgttgagtactgcgaattaccacctgaagcacagtccattaaccctgaaaagaggccagatgaaaattggccatcaaagggtggtattgaattcaaaaactattccacaaaatacagagaaaatttggatccagtgctgaataatattaacgtgaagattgagccatgtgaaaaggttgggattgttggcagaacaggtgcagggaagtctacactgagcctggcattatttagaatactagaacctaccgaaggtaaaattattattgacggcattgatatatccgacataggtctgttcgatttaagaagccatttggcaattattcctcaggatgcacaagcttttgaaggtacagtaaagaccaatttggaccctttcaatcgttattcagaagatgaacttaaaagggctgttgagcaggcacatttaaagcctcatctggaaaaaatgctgcacagtaaaccaagaggtgatgattctaatgaagaggatggcaatgttaatgatattctggatgtcaagattaatgagaacggtagtaacttgtcagtggggcaaagacaactactatgtttggcaagagcgctgctaaaccgttccaaaatattggtccttgatgaagcaacggcttctgtggatatggaaaccgataaaattatccaagacactataagaagagaatttaaggaccgtaccatcttaacaattgcacatcgtatcgacactgtattggacagtgataagataattgttcttgaccagggtagtgtgagggaattcgattcaccctcgaaattgttatccgataaaacgtctattttttacagtctttgtgagaaaggtgggtatttgaaataa

SEQ ID NO: 21 - DNA, 4680 bp, synthetic construct; identical to SEQ ID NO: 20
  misc_feature 1..4680: note = Synthetic: nucleic acid sequence
  source 1..4680: mol_type = other DNA

SEQ ID NO: 22 - DNA, 4680 bp, synthetic construct; identical to SEQ ID NO: 20
  misc_feature 1..4680: note = Synthetic: nucleic acid sequence
  source 1..4680: mol_type = other DNA

SEQ ID NO: 23 - DNA, 4680 bp, synthetic construct; identical to SEQ ID NO: 20
  misc_feature 1..4680: note = Synthetic: nucleic acid sequence
  source 1..4680: mol_type = other DNA

SEQ ID NO: 24 - DNA, 4680 bp, synthetic construct; identical to SEQ ID NO: 20
  misc_feature 1..4680: note = Synthetic: nucleic acid sequence
  source 1..4680: mol_type = other DNA

SEQ ID NO: 25 - DNA, 4680 bp, synthetic construct; identical to SEQ ID NO: 20
  misc_feature 1..4680: note = Synthetic: nucleic acid sequence
  source 1..4680: mol_type = other DNA

SEQ ID NO: 26 - DNA, 4680 bp, synthetic construct; identical to SEQ ID NO: 20
  misc_feature 1..4680: note = Synthetic: nucleic acid sequence
  source 1..4680: mol_type = other DNA

SEQ ID NO: 27 - DNA, 4953 bp, synthetic construct
  misc_feature 1..4953: note = Synthetic: nucleic acid sequence
  source 1..4953: mol_type = other DNA
atgtcaggttcaaattcgaattcaaatctagatgcaataagtgattcatgcccattttggcgctatgatgatattacagagtgtggaagagtgcagtatatcaattactaccttccaataacattggtaggcgtttctctcttgtatttattcaaaaacgcgatccaacattattacagaaagcctcaagaaattaagcctagtgttgcttccgaattattgggctcaaatctcacagaccttccgaatgaaaacaagcctttactatcggagagtacacaagcattatacactaatccggattcgaataagacaggattctctctaaaagaggagcatttctctataaataaagttacacttacggaaattcattccaataagcatgacgctgtgaagatcgtaaggagaaactggcttgaaaaattaagagtgttcttagaatgggttctatgcgccttacaactttgcatctacatttcagtctggtcgaaatacactaatacccaagaggatttcccaatgcacgcatctatctcaggtctaatgttatggtctctactcttgttagtagtgtcattgaggttggcaaacatcaaccagaatataagctggatcaattcaggaccgggaaacttatgggccctttcatttgcatgttatctatcactattctgcggatccgttttgccattgagatctatctatatcggtcatatcacagatgaaattgcatcaacattttataagttgcaattttacctaagtttgacactattcttgttacttttcacctctcaagcgggaaatcggtttgccattatctataaaagtacaccagatataacaccgtctcctgaacctattgtgtcgattgcaagttatatcacttgggcatgggtagataaatttctttggaaagcgcatcaaaattatatcgaaatgaaagatgtttggggtctaatggtggaagactattccattctcgtaataaagagattcaatcattttgttcagaataaaaccaagtctaggacattttcatttaacttaatccactttttcatgaaatttatcgccattcaaggtgcctgggcaacaatttcgtcagttattagttttgttccaacaatgttgctcagacgtattttggagtatgttgaagatcaatcaactgctccattaaatttggcttggatgtatatttttcttatgttccttgccagaattttaactgccatatgtgctgctcaggcgctatttttagggagaagggtttgtatcagaatgaaggctatcataatttctgaaatctactccaaggctttgagaagaaaaatttctccaaattccactaaggagccaactgatgtcgttgatccacaggaattaaatgacaaacaacacgttgatggagatgaagaatcagcaaccactgcaaatcttggtgctatcattaatttgatggcggtggatgctttcaaagtatccgaaatatgtgcgtatttgcactcctttatagaggcgatcatcatgaccattgttgcattattccttttatatcggttaataggctggtctgctttagttggtagtgcaatgattatttgcttcttaccattgaacttcaaacttgccagcttgttagggacactccaaaagaaatccttggcaatcacagataaaagaattcagaaactaaacgaagctttccaggccattcgtattatcaaattcttctcttgggaagagaattttgaaaaggacatacaaaacacaagggatgaagaattaaatatgcttttaaaaaggtctatcgtttgggctctttcttctcttgtttggttcattaccccctctattgtcacatccgcttcttttgcagtctatatttatgtgcaaggccaaactttaactactccggtagcatttactgcactatctctatttgctctactaagaaatccgttagacatgctttctgatatgttgtcttttgttattcaatccaaggtctctttggatagagtccaagaatttttaaatgaagaggagacgaaaaagtatgagcaattaaccgtatcaagaaataaacttgggttgcaaaacgctacttttacatgggataaaaataatcaagatttcaagttaaaaaacctaactattgatttcaaaattgggaaattaaacgttattgtaggtccaactggatctggtaaaacatcattgttaatgggattattgggtgaaatggagctattgaacggaaaagttttcgtcccttcgctcaatcctagggaagagttggttgtagaggccgatggaatgactaattcaatcgcgtactgctcccaagctgcctggttgctaaatgatactgtcaggaacaatattctattcaatgcgccttataatgagaatagatataatgccgtcatctctgcgtgtggtttgaaacgcgacttcgagatcttaagcgctggtgatcagacagagattggcgaaaagggtataacactttctggtggtcaaaaacaaagagtctcgttggccagatcattgtattcttcatcaagacatttgctgttagatgattgtttgagtgccgtagactcgcacacggccttatggatctacgaaaattgtataacaggcccattaatggaaggaagaacatgtgtattggtttctcacaatgttgcattaactttaaaaaatgcagattgggttatcattatggaaaatggtagagtaaaagaacaaggcgaaccagtagaattgctacagaaggggtcccttggggatgactccatggtgaaatcatcaattttgtcccgtacggcgtcctcagttaatatttcagaaactaacagtaagatttctagtggtccgaaggctccagcggaatcggataatgccaatgaggagtccaccacctgtggagatcgttcaaagtcaagcggcaagctaatcgctgaagaaacaaaatcaaacggtgttgtttccctggacgtctataagtggtatgccgtgtttttcggtggatggaagatgatatcatttttgtgtttcattttcttgtttgcccaaatgatcagtatttcacaggcctggtggttgcgtgcttgggcctccaacaacactctaaaagttttctccaaccttggattgcaaacaatgaggccattcgctttgtccttacaaggaaaagaagcttctcctgtgactcttagtgctgttttcccaaatggcagtctaacaacagccacggaaccaaatcactcgaacgcgtattatctatcaatatatttgggtattggtgtattccaggctttatgttcatcttcgaaagcaattataaactttgtggccggtattagagcttccaggaaaatattcaatttattgttgaaaaatgtgttatacgccaagctgagattttttgattctactccaataggaagaataatgaacagattttctaaagacatcgaatcaatagatcaagaattgactccttatatggaaggtgcatttggttccttaatacaatgtgtttccacaattatcgtcattgcatacattactccccaatttttgattgtcgcggcgattgtcatgttattgttttattttgttgcctacttttacatgtcaggagcaagagaattaaagcgtcttgaatcgatgtcacgctctcctattcatcagcacttctctgagactcttgtgggtatcacgactattcgagcattttctgacgagcggcgttttctggttgataatatgaagaaaattgatgataataataggcctttcttttacttatgggtctgtaatagatggctatcttacagaatcgagctgataggcgcccttattgttttggctgcaggtagtttcatcttattgaacataaaatcgatcgattctggtttggccggtatttcattgggtttcgctatacaatttaccgatggtgccctttgggttgttaggttatattccaacgttgaaatgaatatgaattccgtcgaaaggttaaaagagtacaccaccatcgagcaagaaccttctaacgttggtgccttggtacctccttgcgaatggccacaaaatggtaaaatcgaagtcaaggatttatctttacgctatgcagctggtctaccaaaggttataaaaaatgtcacattcaccgtcgattcaaagtgtaaagtaggtattgttggcaggactggtgctggtaaatctactattatcacagcccttttcagattcttagaccctgaaactggttatatcaaaatcgatgacgttgatataacaaccattggtttaaaacgtttgcgccaatctatcactattattccacaggacccaacccttttcaccggtactttgaaaaccaatctcgatccatacaacgaatattcggaagctgaaattttcgaagctctaaaacgtgtcaaccttgtttcctcagaagaacttggtaatccttctacttcggattcaacctcggtacattcagcaaatatgaataagtttttggatttggaaaatgaagtcagtgaaggtggttccaacctctcacaaggacaacgtcaattgatatgtttggcccgttcattattgcggtgtccaaaggtaattctacttgatgaagccacagcttcaatcgattataactcagactctaaaatccaggctactataagggaagaattcagtaatagtaccattctcacgattgctcatcgtttacgatcaattattgattatgataaaatacttgttatggatgctggggaggttaaagaatatgatcatccttactccttattgttgaatcgtgatagtatattctatcatatgtgtgaagatagtggagaattagaagtcttgatacaattagccaaagaatcatttgtcaaaaagctcaatgcaaattga

SEQ ID NO: 28 - protein, 1381 aa, synthetic construct
  REGION 1..1381: note = Synthetic: Fungal_5_muA
MTSPGSEKCTPRSDEDLERSEPQLQRRLLTPFLLSKKVPPIPKEDERKPYPYLKTNPLSQILFWWLNPLLRVGYKRTLDPNDFYYLEHSQDIETTYSNYEMHLARILEKDRAKARAKDPTLTDEDLKNREYPKNAVIKALFLTFKWKYLWSIFLKLLSDIVLVLNPLLSKALINFVDEKMYNPDMSVGRGVGYAIGVTFMLGTSGILINHFLYLSLTVGAHCKAVLTTAIMNKSFRASAKSKHEYPSGRVTSLMSTDLARIDLAIGFQPFAITVPVPIGVAIALLIVNIGVSALAGIAVFLVCIVVISASSKSLLKMRKGANQYTDARISYMREILQNMRIIKFYSWEDAYEKSVVTERNSEMSIILKMQSIRNFLLALSLSLPAIISMVAFLVLYGVSNDKNPGNIFSSISLFSVLAQQTMMLPMALATGADAKIGLERLRQYLQSGDIEKEYEDHEKPGDRDVVLPDNVAVELNNASFIWEKFDDADDNDGNSEKTKEVVVTSKSSLTDSSHIDKSTDSADGEYIKSVFEGFNNINLTIKKGEFVIITGPIGSGKSSLLVALAGFMKKTSGTLGVNGTMLLCGQPWVQNCTVRDNILFGLEYDEARYDRVVEVCALGDDLKMFTAGDQTEIGERGITLSGGQKARINLARAVYANKDIILLDDALSAVDARVGKLIVDDCLTSFLGDKTRILATHQLSLIEAADRVIYLNGDGTIHIGTVQELLESNEGFLKLMEFSRKSESEDEEDVEAANEKDVSLQKAVSVVQEQDAHAGVLIGQEERAVNGIEWDIYKEYLHEGRGKLGIFAIPTIIMLLVLDVFTSIFVNVWLSFWISHKFKARSDGFYIGLYVMFVILSVIWITAEFVVMGNFSSTAARRLNLKAMKRVLHTPMHFLDVTPMGRILNRFTKDTDVLDNEIGEQARMFLHPAAYVIGVLILCIINIPWFAIAIPPLAIPFTFITNFYIASSREVKRIEAIQRSLVYNNFNEVLNGLQTLKAYNATSRFMEKNKRLLNRMNEAYLLVIANQRWISVNLDLVSCCFVFLISMLSVFRVFDINASSVGLVVTSVLQIGGLMSLIMRAYTTVENEMNSVERLCHYANKLEQEAPYIMNETKPRPTWPEHGAIEFKHASMRYREGLPLVLKDLTISVKGGEKIGICGRTGAGKSTIMNALYRLTELAEGSITIDGVEISQLGLYDLRSKLAIIPQDPVLFRGTIRKNLDPFGQNDDETLWDALRRSGLVEGSILNTIKSQSKDDPNFHKFHLDQTVEDEGANFSLGERQLIALARALVRNSKILILDEATSSVDYETDSKIQKTISTVFSHCTILCIAHRLKTILTYDRILVLEKGEVEEFDTPRVLYSKNGVFRQMCERSEITSADFV

SEQ ID NO: 29 - protein, 1381 aa, synthetic construct
  REGION 1..1381: note = Synthetic: Fungal_5_muA2
MTSPGSEKCTPRSDEDLERSEPQLQRRLLTPFLLSKKVPPIPKEDERKPYPYLKTNPLSQILFWWLNPLLRVGYKRTLDPNDFYYLEHSQDIETTYSNYEMHLARILEKDRAKARAKDPTLTDEDLKNREYPKNAVIKALFLTFKWKYLWSIFLKLLSDIVLVLNPLLSKALINFVDEKMYNPDMSVGRGVGYAIGVTFMLGTSGILINHFLYLSLTVGAHCKAVLTTAIMNKSFRASAKSKHEYPSGRVTSLMSTDLARIDLAIGFQPFAITVPVPIGVAIALLIVNIGVSALAGIAVFLVCIVVISASSKSLLKMRKGANQYTDARISYMREILQNMRIIKFYSWEDAYEKSVVTERNSEMSIILKMQSIRNFLLALSLSLPAIISMVAFLVLYGVSNDKNPGNIFSSISLFSVLAQQTMMLPMALATGADAKIGLERLRQYLQSGDIEKEYEDHEKPGDRDVVLPDNVAVELNNASFIWEKFDDADDNDGNSEKTKEVVVTSKSSLTDSSHIDKSTDSADGEYIKSVFEGFNNINLTIKKGEFVIITGPIGSGKSSLLVALAGFMKKTSGTLGVNGTMLLCGQPWVQNCTVRDNILFGLEYDEARYDRVVEVCALGDDLKMFTAGDQTEIGERGITLSGGQKARINLARAVYANKDIILLDDALSAVDARVGKLIVDDCLTSFLGDKTRILATHQLSLIEAADRVIYLNGDGTIHIGTVQELLESNEGFLKLMEFSRKSESEDEEDVEAANEKDVSLQKAVSVVQEQDAHAGVLIGQEERAVNGIEWDIYKEYLHEGRGKLGIFAIPTIIMLLVLDVFTSIFVNVWLSFWISHKFKARSDGFYIGLYVMFVILSVIWITAEFVVMGNFSSTAARRLNLKAMKRVLHTPMHFLDVTPMGRILNRFTKDTDVLDNEIGEQARMFLHPAAYVIGVLILCIINIPWFAIAIPPLAIPFTFITNFYIASSREVKRIEAIQRSLVYNNFNEVLNGLQTLKAYNATSRFMEKNKRLLNRMNEAYLLVIANQRWISVNLDLVSCCFVFLISMLSVFRVFDINASSVGLVVTSVLQIGGLMSLIMRAYTTVENEMNSVERLCHYANKLEQEAPYIMNETKPRPTWPEHGAIEFKHASMRYREGLPLVLKDLTISVKGGEKIGICGRTGAGKSTIMNALYRLTELAEGSITIDGVEISQLGLYDLRSKLAIIPQDPVLFRGTIRKNLDPFGQNDDETLWDALRRSGLVEGSILNTIKSQSKDDPNFHKFHLDQTVEDEGANFSLGERQLIALARALVRNSKILILDEATSSVDYETDSKIQKTISTEFSHCTILCIAHRLKTILTYDRILVLEKGEVEEFDTPRVLYSKNGVFRQMCERSEITSADFV

SEQ ID NO: 30 - protein, 1381 aa, synthetic construct
  REGION 1..1381: note = Synthetic: Fungal_5_muA3
MTSPGSEKCTPRSDEDLERSEPQLQRRLLTPFLLSKKVPPIPKEDERKPYPYLKTNPLSQILFWWLNPLLRVGYKRTLDPNDFYYLEHSQDIETTYSNYEMHLARILEKDRAKARAKDPTLTDEDLKNREYPKNAVIKALFLTFKWKYLWSIFLKLLSDIVLVLNPLLSKALINFVDEKMYNPDMSVGRGVGYAIGVTFMLGTSGILINHFLYLSLTVGAHCKAVLTTAIMNKSFRASAKSKHEYPSGRVTSLMSTDLARIDLAIGFQPFAITVPVPIGVAIALLIVNIGVSALAGIAVFLVCIVVISASSKSLLKMRKGANQYTDARISYMREILQNMRIIKFYSWEDAYEKSVVTERNSEMSIILKMQSIRNFLLALSLSLPAIISMVAFLVLYGVSNDKNPGNIFSSISLFSVLAQQTMMLPMALATGADAKIGLERLRQYLQSGDIEKEYEDHEKPGDRDVVLPDNVAVELNNASFIWEKFDDADDNDGNSEKTKEVVVTSKSSLTDSSHIDKSTDSADGEYIKSVFEGFNNINLTIKKGEFVIITGPIGSGKSSLLVALAGFMKKTSGTLGVNGTMLLCGQPWVQNCTVRDNILFGLEYDEARYDRVVEVCALGDDLKMFTAGDQTEIGERGITLSGGQKARINLARAVYANKDIILLDDALSAVDARVGKLIVDDCLTSFLGDKTRILATHQLSLIEAADRVIYLNGDGTIHIGTVQELLESNEGFLKLMEFSRKSESEDEEDVEAANEKDVSLQKAVSVVQEQDAHAGVLIGQEERAVNGIEWDIYKEYLHEGRGKLGIFAIPTIIMLLVLDVFTSIFVNVWLSFWISHKFKARSDGFYIGLYVMFVILSVIWITAEFVVMGNFSSTAARRLNLKAMKRVLHTPMHFLDVTPMGRILNRFTKDTDVLDNEIGEQARMFLHPAAYVIGVLILCIINIPWFAIAIPPLAILFTFITNFYIASSREVKRIEAIQRSLVYNNFNEVLNGLQTLKAYNATSRFMEKNKRLLNRMNEAYLLVIANQRWISVNLDLVSCCFVFLISMLSVFRVFDINASSVGLVVTSVLQIGGLMSLIMRAYTTVENEMNSVERLCHYANKLEQEAPYIMNETKPRPTWPEHGAIEFKHASMRYREGLPLVLKDLTISVKGGEKIGICGRTGAGKSTIMNALYRLTELAEGSITIDGVEISQLGLYDLRSKLAIIPQDPVLFRGTIRKNLDPFGQNDDETLWDALRRSGLVEGSILNTIKSQSKDDPNFHKFHLDQTVEDEGANFSLGERQLIALARALVRNSKILILDEATSSVDYETDSKIQKTISTEFSHCTILCIAHRLKTILTYDRILVLEKGEVEEFDTPRVLYSKNGVFRQMCERSEITSADFV